SEQUENCE LISTING (39 sequences)

SEQ ID NO: 1 - protein, 1164 aa, Homo sapiens
MAQQANVGELLAMLDSPMLGVRDDVTAVFKENLNSDRGPMLVNTLVDYYLETSSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSKDHELDPRRWKRLETHDVVIECAKISLDPTEASYEDGYSVSHQISARFPHRSADVTTSPYADTQNSYGCATSTPYSTSRLMLLNMPGQLPQTLSSPSTRLITEPPQATLWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSIFTPSPCKIPPPTRVGFGSGQPPPYDHLFEVALPKTAHHFVIRKTEELLKKAKGNTEEDGVPSTSPMEVLDRLIQQGADAHSKELNKLPLPSKSVDWTHFGGSPPSDEIRTLRDQLLLLHNQLLYERFKRQQHALRNRRLLRKVIKAAALEEHNAAMKDQLKLQEKDIQMWKVSLQKEQARYNQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSARGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHPSPPTPDSVGQLHIMDYNETHHEHS

SEQ ID NO: 2 - DNA, 3529 bp, Homo sapiens
gtacaaaaaagcagaagggccgtcaaggcccaccatggcccaacaagcaaatgtcggggagcttcttgccatgctggactcccccatgctgggtgtgcgggacgacgtgacagctgtctttaaagagaacctcaattctgaccgtggccctatgcttgtaaacaccttggtggattattacctggaaaccagctctcagccggcattgcacatcctgaccaccttgcaagagccacatgacaagcacctcttggacaggattaacgaatatgtgggcaaagccgccactcgtttatccatcctctcgttactgggtcatgtcataagactgcagccatcttggaagcataagctctctcaagcacctcttttgccttctttactaaaatgtctcaagatggacactgacgtcgttgtcctcacaacaggcgtcttggtgttgataaccatgctaccaatgattccacagtctgggaaacagcatcttcttgatttctttgacatttttggccgtctgtcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccaaggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgatgttgttaaatatgccagggcagctacctcagactctgagttccccatcgacacggctgataactgaaccaccacaagctactctttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacaccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtattgaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcttcactcccagtccttgtaaaattccacctccgacgagagtgggctttggaagcgggcagcctcccccgtatgatcatctttttgaggtggcattgccaaagacagcccatcattttgtcatcaggaagactgaggagctgttaaagaaagcaaaaggaaacacagaggaagatggtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataatgctgccatgaaagatcagttgaagttacaagagaaggacatccagatgtggaaggttagtctgcagaaagaacaagctagatacaatcagctccaggagcagcgtgacactatggtaaccaagctccacagccagatcagacagctgcagcatgaccgagaggaattctacaaccagagccaggaattacagacgaagctggaggactgcaggaacatgattgcggagctgcggatagaactgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctctatttggaacaactgcagaacaagcactcagataccacaaaggaagtagaaatgatgaaagccgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaatgacgggtgctcagattccatggtagggcacaatgaagaggcatctggccacaacggtgagaccaagacccccaggcccagcagcgcccggggcagtagtggaagcagaggtggtggaggcagcagcagcagcagcagcgagctttctaccccagagaaacccccacaccagagggcaggcccattcagcagtcggtgggagacgactatgggagaagcgtctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaacctagatggccctcacccgtctcccccgaccccggacagtgttggacagctacatatcatggactacaatgagactcatcatgaacacagctag

SEQ ID NO: 3 - protein, 1784 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTVSSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWEVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSMKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTDSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVMVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 4 - DNA, 5352 bp, Homo sapiens
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgtgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgactacatggagccagagagaggctctgagaagaagaccagcggccccctttctcctcccacagggcctcctggcccggcgcctgcaggccccgccgtgcggctggggtccgtgccctactccctgctcttccgcgtcctgctgcagtgcttgaagcaggagtctgactgggaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtcttttgatgacacccccgagaaggacagcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcatgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatggtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 5 - protein, 1161 aa, Mus musculus
MAQLANIGELLSMLDSSTLGVRDDVTAIFKESLNSERGPMLVNTLVDYYLETNSQPVLHILTTLQEPHDKHLLDKINEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPTEASYEDGYSVSHQLSACFPYRSADVTTSPYVDTQNSYGGSTSTPSSSSRLMLFSPPGQLPQSLSSPSTRLLPEPLQASLWSPSAVCGMTTPPTSPGNVPADLSHPYSKAFGTTAGGKGTPSGTPATSPPPAPPCPQDDCVHGSAAQASATAPRKEERADSSRPYLHRQSNDRGLEDPPGSKGSVTLRNLPDFLGDLASEEDSIEKDKEEAAISKELSEITTAEADPVVPRGGFDSPFYRDSLSGSQRKTHSAASGTQGSSVNPEPLHSSLDKHGPDTPKQAFTPIDPPSGSADVSPAGDRDRQTSLETSILTPSPCKIPPQRGVSFGSGQLPPYDHLFEVALPKTACHFVSKKTEELLKKVKGNPEEDCVPSTSPMEVLDRLIEQGAGAHSKELSRLSLPSKSVDWTHFGGSPPSDELRTLRDQLLLLHNQLLYERFKRQQHALRNRRLLRKVIRAAALEEHNAAMKDQLKLQEKDIQMWKVSLQKEQARYSQLQEQRDTMVTQLHSQIRQLQHDREEFYNQSQELQTKLEDCRNMIAELRVELKKANNKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLRKLEEDRAEAAEAAEERLDCCSDGCTDSLVGHNEEASGHNGETRTSRPGGTRASCGGRVTGGSSSSSSELSTPEKPPSQRFSSRWEPALGEPSSSIPTTVGSLPSSKSFLGMKARELFRNKSESQCDEDSVTMSSSSLSETLKTELGKDSGTENKTSLSLDAPHPSSPNSDNVGQLHIMDYNETHPEHS

SEQ ID NO: 6 - protein, 1814 aa, Mus musculus
MAKPTSKDSGLKEKFKILLGLGTSRPNPRCAEGKQTEFIITSEILRELSGECGLNNRIRMIGQICDVAKTKKLEEHAVEALWKAVSDLLQPERPPEARHAVLTLLKAIVQGQGDRLGVLRALFFKVIKDYPSNEDLHERLEVFKALTDNGRHITYLEEELAEFVLQWMDVGLSSEFLLVLVNLVKFNSCYLDEYIASMVHMICLLCIRTVSSVDIEVSLQVLDAVVCYNCLPAESLPLFIITLCRTINVKELCEPCWKLMRNLLGTHLGHSAIYNMCRIMEDRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELKTIVHDLLTTVEELCDQNEFHGSQERYYELVESYADQRPESSLLNLISYRAQSIHPAKDGWIQNLQLLMERFFRNECRSAVAIKVLDVLSFVLLIIRQFYEEELINSVVISQLSHIPEDKDHQVRKLATQLLVDLAEGCHTHHFNSLLDIIEKVMARSLSPPPELEERDLAVHSASLEDVKTAVLGLLVILQTKLYTLPASHATRVYESLISHIQLHYKHGYSLPIASSIRLQAFDFLLLLRADSLHRLGLPNKDGVVRFSPYCLCDCMELDRASEKKASGPLSPPTGPPSPVPMGPAVRLGYLPYSLLFRVLLQCLKQESDWKVLKLVLSRLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFVPEQYASVFAISLPYTNPSKFNQYIVCLAHHVIAMWFIRCRLPFRKDFVPYITKGLRSNVLLSFDDTPEKDSFRARSTSLNERPKSLRIARAPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSDSSSDPSTHVRQTKEAPAKLESQAGQQVSRGARDRVRSMSGGHGLRVGVLDTSAPYSPGGSASLGPQTAVAAKPEKPPAGAQLPTAEKTNLAAYVPLLTQGWAEILVRRPTGNTSWLMSLENPLSPFSSDINNMPLQELSNALMAAERFKEHGHAPVQVIVSATGCTAKPPTLPRSNTVASFSSLYQPSCQGQLHRSVSWADSAMVLEEGSPGETQVPVEPPELEDFEAALGTDRHCQRPDTYSRSSSASSQEEKSHLEELAAGGIPIERAISSEGARPAVDLSFQPSQPLSKSSSSPELQTLQDILGDLGDKIDIGRLSPEAKVRSQSGILDGEAATWSATGEESRITVPPEGPLPSSSPRSPSGLRPRGYTISDSAPSRRGKRVERDNFKSRAAASSAEKVPGINPSFVFLQLYHSPFFGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKQGQFNFVHVIITPLDYKCNLLTLQCRKDGPACKCEWWRQPGEIVVWALPVVMELTVTILLCHLQMASQVHHSRSNPTDIYPSKWIARLRHIKRLRQRIREEVHYSNPSLPLMHPPAHTKAPAQAPEATPTYETGQRKRLISSVDDFTEFV

SEQ ID NO: 7 - DNA, 5532 bp, Mus musculus
gtcgggggtgtgcgcccttccctgggtgcaggaggacctcggcgcgaaaggcgcgcgcgggagcagttctctagtactgccctgcccaccatggccaaaccaacaagcaaagattccggcttgaaggagaagttcaagatactgttgggattgggaacatcgaggccaaatcccaggtgtgcagaaggcaaacagactgagtttatcatcacatcggaaatcttgagagaactgagtggtgaatgcggcctcaacaatcgcatccgaatgatagggcagatctgtgatgtggcaaaaactaagaagcttgaggagcatgcagtggaggcactttggaaggctgtctcagacttgctacagccagagcggccaccagaggcccggcatgcagttctcaccttattgaaggccattgtacagggacagggtgatcgtttgggagttctcagagccctcttcttcaaagtgatcaaggattacccctccaatgaagacctccatgaaaggctagaagtttttaaggccctcacagacaatgggaggcacattacctatttggaagaagaactggcagagtttgtcctgcagtggatggatgttggcttgtcctcagaattccttctggtacttgtcaacctggtcaagttcaacagctgttaccttgacgaatacattgcatcaatggttcacatgatttgtctgctatgcatccggacagtgtcctctgtggacattgaggtgtccttgcaagtgctggatgctgtggtctgctacaactgcttaccagccgagagcctgcctctgttcattatcaccctgtgccgcaccatcaatgtcaaggagctgtgtgagccttgctggaagttgatgcgtaaccttctgggcacccatctgggccacagcgccatctacaacatgtgccgcatcatggaggacagatcctacatggaagatgccccactgctgagaggagctgtgttctttgtagggatggcactctggggagctcaccggctctactctctcaagaactcccccacatctgtgctgccgtctttttatgaggctatgacctgtcccaatgaggtggtgtcatatgagattgttctgtccataacaagactcatcaagaagtataggaaggagctccaggctgtgacatgggatattctgctggacatcattgaacgactacttcagcaactccagaacctggacagcccggaactcaagaccatcgtccatgacctgctgaccactgtggaggagctatgtgaccagaacgagttccatggctcacaggaaagatactatgaactggtggagagctatgcagaccagagacctgaatcctctctcttaaacttgatatcctacagagcccagtccatccaccccgccaaggatggctggatccagaacttgcagttgttgatggagaggttcttcaggaatgagtgccgcagcgccgtggccatcaaggtgttggatgttctgtccttcgtgctgctgatcatcaggcagttctatgaggaggagctgattaactcggtggtcatctcgcagctctcccacattcccgaggataaggaccatcaggtccgaaagctggctactcagctgctggtggacctggcagaggggtgccacacccaccacttcaacagtctgctggacatcattgaaaaggtgatggcgcgctcactctctccacccccggagctggaagaaagggacctggccgtgcactcggcctccctggaggacgtgaagaccgcggtcctagggctcctggtcatccttcagaccaagctctacaccttgcctgccagccacgccactcgagtgtatgagagcctcattagtcacatccagctccattacaagcacggctactccctgcccattgctagcagcatccgactgcaggcctttgacttcctgctgctactgcgggctgactcgctgcatcgactgggcctgcccaacaaggatggggtcgtgagattcagcccttactgcctctgtgactgcatggaactggatagagcctcagagaagaaagccagtgggcccctttcacctccaactgggccccctagccctgtgcctatgggccctgctgtgcggctcggctacctaccctactccctgctcttccgtgtcctgttgcagtgtttgaagcaggagagcgactggaaggtgctgaagctggtgctcagcaggctgccggagtcactgcgctacaaagtcctcatcttcacctccccctgcagtgtcgaccagctgtcttctgccctctgctccatgctttcagctccaaagacccttgagcggctccgaggtaccccagaaggcttctcgagaactgacctgcacttggccgtggttcccgtgctaacagcattaatatcttatcacaactatttggacaagaccagacagcgtgagatggtgtactgcctggaacaaggccttatctaccgctgcgccagccagtgcgtggtagccttggccatctgcagtgtggagatgcccgacatcatcatcaaggcgcttcctgtcctggtggtgaagctcacacacatttctgccacagccagcatggccatcccgctcctggagttcctgtctactttggccaggctgccccacctctacaggaactttgtgccagagcagtatgccagcgtgtttgccatctccttgccatacaccaacccctccaagttcaaccagtacattgtgtgtctggctcaccacgtcatagccatgtggttcatcaggtgccgactgccctttcggaaggatttcgtcccttatatcactaagggtttgcgttccaatgtcctcctgtcttttgatgatacccctgagaaggacagtttcagagcacggagcaccagtcttaatgagagacccaaaagtttgaggatagccagagcccccaaacaaggcctgaataactctccacctgtgaaagaattcaaagagagctgtgcagccgaggccttccggtgccgcagcatcagtgtatctgaacatgtggtccgcagcagaatacagacatcccttacgagcgccagcctggggtctgcggatgagaactctatggcccaggctgatgacaacttgaagaatctccacttggagctcacagaaacatgtctggacatgatggcccgatatgtgttctccaatttcactgcagtccccaagaggtcccctgtgggagagtttctcctggcagggggtaggaccaaaacctggctggttggaaacaagcttgtcactgtgacaacaagtgtgggaactggtacacggtcgctgctgggcctggactctggggacctgcagggtggctcggattcaagctctgatcctagcacacatgtgaggcagacaaaggaagcaccggccaaactggagtcccaggctgggcagcaggtgtcccgtggggcccgggaccgggtccgctccatgtcggggggccatggccttcgagttggtgtcctggatacttcagctccctattccccaggtggctctgcttctctgggaccacagacggcggtggcagcgaagcccgagaagccccctgcaggagcccagcttccaacagcagagaagacgaatctggcagcctatgtgcctttgttaacccagggctgggcagaaatcttagtccgcagacccacaggaaacaccagctggctgatgagcttggagaacccgctcagccccttctcctcagacatcaacaacatgcccctgcaagagctgtccaatgcccttatggctgctgaacgtttcaaggagcacggacacgcccctgtacaagtcattgtcagtgccacgggctgcaccgccaagcctcctactctcccacgctctaatacagtggcttctttctcctccctgtaccagcccagctgccaaggacagctgcacaggagcgtttcctgggcagactcagccatggtccttgaggagggaagtccaggagagactcaggtgccagtggagccccccgaattggaggatttcgaggcggcgttaggtacagacaggcactgccagcggcctgacacctacagccggtcatcctcagcatctagccaggaagaaaagtcccatttggaggagcttgctgcagggggtatccccattgagcgggccatctcctctgagggggctcggcctgccgtggacctctccttccaaccctcacagcctttgagcaagtctagctcttctccggagttgcagaccttacaggacatccttggagacctaggggacaagattgatattggacggctgagtcctgaggctaaggtccggtcccagtcagggatcctggatggggaagcagctacctggtcagctacgggtgaagagagccgtatcacagtcccacctgaaggtcctctgccttccagttctccccgctcccccagtggcctccggccccgaggctataccatctctgattccgctccatcacgaaggggaaagagggtagaaagggataacttcaagagcagagctgcggcctccagtgctgagaaggtgccaggcatcaaccctagctttgtgttcctacagctctaccattcgcccttttttggtgatgagtccaataagcccatcctgttgcccaatgagtcctttgaacggtcagtacagctgcttgaccagattccatcctatgacactcacaagattgctgtcctgtatgtgggagaaggccagagcagcagtgagctggccatcctgtcaaatgagcatggctcttacaggtacacggagtttctgacaggcctgggtcggcttattgagctcaaggactgccaaccagacaaggtgtacttaggtggactggatgtatgtggcgaggatggacagttcacctactgctggcatgatgacatcatgcaagctgttttccacattgccaccctgatgcccaccaaggatgtggacaagcaccgctgtgacaagaaacggcacctgggcaatgactttgtttctatcatctacaatgattctggtgaggacttcaaactgggcaccattaagcagggccagttcaactttgtccatgtgatcatcacaccgctggactacaagtgcaacctattgaccctgcagtgcaggaaagatggccctgcatgcaaatgtgagtggtggcggcagcctggggagatagtcgtgtgggcgctccctgttgtgatggagctcactgtcaccatcctgctctgtcacctccagatggcctcacaggtacaccacagccgatccaaccccactgacatctatccctccaagtggatcgcaagactccgccacattaagcgtctccgccagaggatccgtgaagaggtgcactattccaacccaagcttgcctctgatgcaccctccagcccacaccaaagccccagctcaagcccctgaggctacacccacctatgaaacaggccagcggaagcgcctcatttcctccgtggatgacttcacagagtttgtg

SEQ ID NO: 8 - DNA, 228 bp, Human cytomegalovirus
gctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaac

SEQ ID NO: 9 - protein, 14 aa, Artificial Sequence
  V5 peptide tag
GKPIPNPLLGLDST

SEQ ID NO: 10 - DNA, 42 bp, Artificial Sequence
  V5 peptide tag
ggaaagccgatcccaaaccctctattaggtctggactccacc

SEQ ID NO: 11 - DNA, 7604 bp, Artificial Sequence
  AAV9-hTSC1-V5 vector
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagcatgcctgcaggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaaggctagagtacttaatacgactcactataggctagcctcgagaattcacgcgtgccgccaccatggcccaacaagcaaatgtcggggagcttcttgccatgctggactcccccatgctgggtgtgcgggacgacgtgacagctgtctttaaagagaacctcaattctgaccgtggccctatgcttgtaaacaccttggtggattattacctggaaaccagctctcagccggcattgcacatcctgaccaccttgcaagagccacatgacaagcacctcttggacaggattaacgaatatgtgggcaaagccgccactcgtttatccatcctctcgttactgggtcatgtcataagactgcagccatcttggaagcataagctctctcaagcacctcttttgccttctttactaaaatgtctcaagatggacactgacgtcgttgtcctcacaacaggcgtcttggtgttgataaccatgctaccaatgattccacagtctgggaaacagcatcttcttgatttctttgacatttttggccgtctgtcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccaaggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgatgttgttaaatatgccagggcagctacctcagactctgagttccccatcgacacggctgataactgaaccaccacaagctactctttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacaccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtattgaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcttcactcccagtccttgtaaaattccacctccgacgagagtgggctttggaagcgggcagcctcccccgtatgatcatctttttgaggtggcattgccaaagacagcccatcattttgtcatcaggaagactgaggagctgttaaagaaagcaaaaggaaacacagaggaagatggtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataatgctgccatgaaagatcagttgaagttacaagagaaggacatccagatgtggaaggttagtctgcagaaagaacaagctagatacaatcagctccaggagcagcgtgacactatggtaaccaagctccacagccagatcagacagctgcagcatgaccgagaggaattctacaaccagagccaggaattacagacgaagctggaggactgcaggaacatgattgcggagctgcggatagaactgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctctatttggaacaactgcagaacaagcactcagataccacaaaggaagtagaaatgatgaaagccgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaatgacgggtgctcagattccatggtagggcacaatgaagaggcatctggccacaacggtgagaccaagacccccaggcccagcagcgcccggggcagtagtggaagcagaggtggtggaggcagcagcagcagcagcagcgagctttctaccccagagaaacccccacaccagagggcaggcccattcagcagtcggtgggagacgactatgggagaagcgtctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaacctagatggccctcacccgtctcccccgaccccggacagtgttggacagctacatatcatggactacaatgagactcatcatgaacacagcggaaagccgatcccaaaccctctattaggtctggactccacctaagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 12 - protein, 1163 aa, Rattus norvegicus
MAQLANIGELLSMLDSSTLGVRDDVTTIFKESLNSERGPMLVNTLVDYYLETNSQPVLHILTTLQEPHDKHLLDKMNEYVGKAATRLSILSLLGHVVRLQPSWKHKLSQAPLLPSLLKCLKMDTDVVVLTTGVLVLITMLPMIPQSGKQHLLDFFDIFGRLSSWCLKKPGHVTEVYLVHLHASVYALFHRLYGMYPCNFVSFLRSHYSMKENVETFEEVVKPMMEHVRIHPELVTGSKDHELDPRRWKTLETHDVVIECAKISLDPTEASYEDGDAVSHQLSACFPHRSADVTTSSYVDTQNSYGGATSTPSSTSRLMLFSTPGQLPQSLSSLSTRPLPEPLQASLWSPSAVCGMTTPPTSPGNVPADLSHPYSKAFGTTTGGKGTPSGTPATSPPPAPPCPQDDCAHGPASQASATPPRKEERADSSRPYLPRQQDVPSDRGLEDLPGSKGSVTLRNLPDFLGDLASEEDSIEKDKEEAAISKELSEITTAEADPVAPRGGFDSPFYRDSLSGSQRKTHSAASGTQGFSVNPEPLHSSLDKHGPDTPKQAFTPIDPPSGSADASPAGDRDRQTSLETSILTPSPCKIPPQRGVSFGSGQLPPYDHLFEVALPKTACHFVSKKTEELLKKAKGNPEEDCVPSTSPMEVLDRLLEQGAGAHSKELSRLSLPSKSVDWTHFGGSPPSDEIRTLRDQLLLLHNQLLYERFKRQQHALRNRRLLRKVIRAAALEEHNAAMKDQLKLQEKDIQMWKVSLQKEQARYSQLQQQRDTMVTQLHSQIRQLQHDREEFYNQSQELQTKLEDCRSMIAELRVELKKANSKVCHTELLLSQVSQKLSNSESVQQQMEFLNRQLLVLGEVNELYLEQLQSKHPDTTKEVEMMKTAYRKELEKNRSHLLQQNQRLDASQRRVLELESLLAKKDHLLLEQKKYLEDVKSQASGQLLAAESRYEAQRKITRVLELEILDLYGRLEKDGRLQKLEEDRAEAAEAAEERLDCCTDGCSDSLLGHNEEAAGHNGETRTSRPGGTRASCGGRVTGGSSSSSSELSTPEKPPNQRFSSRWEPTMGEPSSSIPTTVGSLPSSKSFLGMKTRELFRNKSESQCDEDGMTMSSFSETLKTELGKDSAGMENKTPPSLDAPHPSSPSSDSMGQLHIMDYNETHHEHS

SEQ ID NO: 13 - DNA, 3492 bp, Rattus norvegicus
atggcccagctagccaacattggggagctactctccatgctggactcttccacactgggtgtgcgtgatgacgtgacaaccatcttcaaggagtccctcaattctgaacgtgggcctatgcttgtaaacacgttggtggattattacctggaaaccaattctcagccggtcttgcacatcctgaccaccctgcaagagccacatgataagcacctcttggacaaaatgaatgagtatgtaggcaaagctgccacccgcttatccatcctctcgctgctggggcatgttgtgaggctgcagccatcttggaagcataagctctctcaagcacctcttctgccttctttactgaaatgtctcaagatggacactgatgttgtggttctcacaactggtgtcttggtgttgatcaccatgctaccgatgatcccgcagtcagggaagcagcaccttctcgacttctttgacatctttggccgtctttcatcatggtgcctgaagaaaccaggccatgtgacagaagtctacctggtccacctccatgccagtgtttatgccctcttccatcgcctttatggaatgtacccttgtaacttcgtctccttcctgcgctctcactacagcatgaaggaaaacgtggagacttttgaagaagtggtcaagccaatgatggagcatgtgcgaattcacccggaattagtgactggatccaaggaccatgaactggaccctcgaaggtggaagacattagaaactcatgatgttgtaatagagtgtgccaaaatctctctggaccctaccgaagcctcatatgaagatggcgatgctgtgtctcaccagctctctgcctgcttccctcaccgttcagctgatgtcaccaccagctcttacgtggacacacagaatagctatgggggcgctacttccaccccttcctccacctctcggctgatgttgttcagtacccccgggcagctacctcagagtttgagttcactatcaacacggccgttacctgagccgctgcaagctagtctttggagcccatctgcagtctgtggtatgaccactcctcctacgtctcctggaaatgtcccggctgatttgtcacatccatacagtaaagcctttggtactaccactggtggaaaaggaactccttcaggaaccccagcgacctctcctcccccagccccgccttgtccccaagacgactgtgcgcacggtccagcctcccaggcctcagccacaccccccaggaaggaagaaagagcagattcttcaaggccttacctacccagacagcaggatgttccgagtgaccgcggattagaggatctacctggaagcaaaggttccgtcactctgaggaatctaccagatttcctaggtgacctggcttcagaggaagacagtatagagaaagataaggaagaagctgcgatatctaaagagctttctgagatcaccactgcagaggcggatcctgtagctcctcgagggggctttgactctcccttctaccgagacagtctctctggttctcagcggaagactcactcagcagcctctgggactcagggcttcagcgtgaaccctgagcctttgcactcctccctggacaaacatgggcctgacacaccaaagcaagcctttactcccatagacccaccctctggcagtgctgatgccagtcccgctggggacagggatcgccagacttctctggagaccagtatcctcactcccagcccttgcaaaattccacctcagaggggagtgagctttggaagtgggcagcttcccccatatgatcatctctttgaggtggccttgccaaagactgcctgtcactttgtcagcaaaaagactgaagagctgttgaagaaagcgaaaggaaaccctgaggaagactgtgtgccctctacctccccaatggaagtactggacagactgctagagcagggagcaggggcacacagcaaggaactgagcaggttgtccttgcccagcaagtctgttgactggacccactttggaggctctcccccctcagatgagatccggaccctccgagaccagttgctcttactgcacaatcagctgctgtacgaacgctttaagcggcagcagcatgcactgaggaacagaaggctgctgcgcaaggtgatccgagctgcggccctggaggagcacaacgcagcgatgaaagatcagttgaagttacaagagaaggacatccagatgtggaaggtgagtctgcagaaagaacaagcccgatacagtcagcttcagcaacaacgtgacaccatggtgacccagctgcatagccaaatcagacagctgcagcatgaccgagaagaattctacaaccagagtcaggagttacagacgaagctggaggactgcagaagcatgatcgcggagcttcgagtggagctgaagaaggccaacagcaaggtgtgccacaccgagctgctgctcagccaggtctctcagaagctctccaatagtgagtcagtgcagcagcagatggagttcttgaataggcagctcctggtgctcggggaggtcaatgagctgtacctggagcagctgcagagcaagcatcctgacaccaccaaggaagtagaaatgatgaaaactgcttatcggaaagaactagagaaaaacagaagccaccttctccagcagaaccagaggctggatgcctcacagaggcgagttttagaactggaatctcttctggccaagaaagaccaccttctcctagagcagaagaagtaccttgaggatgtcaagagccaggcgagtggacagctgctggctgcagagagcaggtatgaggctcagagaaagatcacccgggtgttggaactggagatcctagacttgtatggtagattggagaaagatggccgcctacagaaactagaagaggacagagcagaggcagcagaggcagctgaagagaggcttgactgttgtactgatggatgctcagattccttgttaggacataatgaagaggctgctggtcacaatggtgagaccaggacctccagacctggtggcacccgggccagctgtggaggtagagtcactggaggcagcagcagcagcagcagtgagctttccactccagagaaacccccgaaccagaggttcagcagccggtgggagcccaccatgggtgagccctccagcagcatccccaccactgttggctcacttcccagttccaaaagcttcttgggcatgaagacccgggagctgttccgtaataagagtgagagccagtgtgatgaggatggcatgaccatgagtagcttttctgagaccctgaagacagaactgggcaaagactcggcgggcatggaaaacaagactcccccaagtctagacgccccacacccatcttccccaagctcagacagtatggggcagctccatatcatggactacaatgagactcatcacgaacacagctga

SEQ ID NO: 14 - protein, 1164 aa, Pongo abelii
MAQQANVGELLAMLDSPMLGVRDDVTAVFKENLNSDRGPMLVNTLVDYYLETNSQPALHILTTLQEPHDKHLLDRINEYVGKAATRLSILSLLGHVIRLQPSWKHKLSQAPLLPSLLKCLKMDTDVVVLTTGVLVLITMLPMIPQSGKQHLLDFFDIFGRLSSWCLKKPGHVAEVYLVHLHASVYALFHRLYGMYPCNFVSFLRSHYSMKENLETFEEVVKPMMEHVRIHPELVTGSRDHELDPRRWKRLETHDVVIECAKISLDPTEASYEDGYSVSHQISARFPHRSADVTTSPYADTQNSYGCATSTPYSTSRLTLLNTPGQLPQTLSSPSTRLITEPPQATFWSPSMVCGMTTPPTSPGNVPPDLSHPYSKVFGTTAGGKGTPLGTPATSPPPAPLCHSDDYVHISLPQATVTPPRKEERMDSARPCLHRQHHLLNDRGSEEPPGSKGSVTLSDLPGFLGDLASEEDSIEKDKEEAAISRELSEITTAEAEPVVPRGGFDSPFYRDSLPGSQRKTHSAASSSQGASVNPEPLHSSLDKLGPDTPKQAFTPIDLPCGSADESPAGDRECQTSLETSILTPSPCKIPPPTRVGFGSGQPPPYDHLFEVALPKTAHHFIIRKTEELLKKAKGNTEEDCVPSTSPMEVLDRLIQQGADAHSKELNKLPLPSKSVDWTHFGGSPPSDEIRTLRDQLLLLHNQLLYERFKRQQHALRNRRLLRKVIKAAALEEHNTAMKDQLKLQEKDIQMWKVSLQKEQARYSQLQEQRDTMVTKLHSQIRQLQHDREEFYNQSQELQTKLEDCRNMIAELRIELKKANNKVCHTELLLSQVSQKLSNSESVQQQMEFLNRQLLVLGEVNELYLEQLQNKHSDTTKEVEMMKAAYRKELEKNRSHVLQQTQRLDTSQKRILELESHLAKKDHLLLEQKKYLEDVKLQARGQLQAAESRYEAQKRITQVFELEILDLYGRLEKDGLLKKLEEEKAEAAEAAEERLDCCNDGCSDSMVGHNEEASGHNGETKTPRPSSTRGSSGSRGGGGSSSSSSELSTPEKPPHQRAGPFSSRWETTMGEASASIPTTVGSLPSSKSFLGMKARELFRNKSESQCDEDGMTSSLSESLKTELGKDLGVEAKIPLNLDGPHTPPPNPDSVGQLHIMDYNETHHEHS

SEQ ID NO: 15 - DNA, 3492 bp, Pongo abelii
atggcccaacaagcgaatgtcggggagcttcttgccatgctggactcccccatgctgggtgtgcgggacgacgtgacagctgtctttaaagagaacctcaattctgaccgtggtcctatgcttgtaaacaccttggtggattattacctggaaaccaactctcagccagcattgcacatcctgaccaccttgcaagagccacatgacaagcacctcttggacaggattaacgaatatgtgggcaaagctgccactcgtttatccatcctctcgttactgggtcatgtcataagactgcagccatcttggaagcataaactctctcaagcacctcttttgccttctttactaaaatgtctcaagatggacactgatgtcgttgtcctcacaacaggcgtcttggtgttgataaccatgctaccaatgattccacagtctgggaaacagcatcttcttgatttctttgacatttttggccgtctatcatcatggtgcctgaagaaaccaggccacgtggcggaagtctatctcgtccatctccatgccagtgtgtacgcactctttcatcgcctttatggaatgtacccttgcaacttcgtctcctttttgcgttctcattacagtatgaaagaaaacctggagacttttgaagaagtggtcaagccaatgatggagcatgtgcgaattcatccggaattagtgactggatccagggaccatgaactggaccctcgaaggtggaagagattagaaactcatgatgttgtgatcgagtgtgccaaaatctctctggatcccacagaagcctcatatgaagatggctattctgtgtctcaccaaatctcagcccgctttcctcatcgttcagccgatgtcaccaccagcccttatgctgacacacagaatagctatgggtgtgctacttctaccccttactccacgtctcggctgacgttgttaaatacgccagggcagctacctcagactctgagttccccatcgacacggctgataactgaaccaccacaagctactttttggagcccatctatggtttgtggtatgaccactcctccaacttctcctggaaatgtcccacctgatctgtcacacccttacagtaaagtctttggtacaactgcaggtggaaaaggaactcctctgggaaccccagcaacctctcctcctccagccccactctgtcattcggatgactacgtgcacatttcactcccccaggccacagtcacgccccccaggaaggaagagagaatggattctgcaagaccatgtctacacagacaacaccatcttctgaatgacagaggatcagaagagccacctggcagcaaaggttctgtcactctaagtgatcttccagggtttttaggtgatctggcctctgaagaagatagtatagaaaaagataaagaagaagctgcaatatctagagaactttctgagatcaccacagcagaggcagagcctgtggttcctcgaggaggctttgactctcccttttaccgagacagtctcccaggttctcagcggaagacccactcggcagcctccagttctcagggcgccagcgtgaaccctgagcctttacactcctccctggacaagcttgggcctgacacaccaaagcaagcctttactcccatagacctgccctgcggcagtgctgatgaaagccctgcgggagacagggaatgccagacttctttggagaccagtatcctcactcccagtccttgtaaaattccacctccaacgagagtgggctttggaagcgggcagcctcccccgtatgatcatctttttgaggtggcattgccaaagacagcccatcattttatcatcaggaagactgaggagctgttaaagaaagcaaaaggaaacacagaggaagattgtgtgccctctacctccccaatggaagtgctggacagactgatacagcagggagcagacgcgcacagcaaggagctgaacaagttgcctttacccagcaagtctgtcgactggacccactttggaggctctcctccttcagatgagatccgcaccctccgagaccagttgcttttactgcacaaccagttactctatgagcgttttaagaggcagcagcatgccctccggaacaggcggctcctccgcaaggtgatcaaagcagcagctctggaggaacataataccgccatgaaagatcagttgaagttacaagagaaggacatccagatgtggaaggttagtctgcagaaagaacaagctagatacagtcagcttcaggagcagcgtgacactatggtaaccaagctccacagccagatcagacagctgcagcatgaccgagaggaattctacaaccagagccaggaattacagacgaagctggaggactgcaggaacatgattgcagagctgcggatagaattgaagaaggccaacaacaaggtgtgtcacactgagctgctgctcagtcaggtttcccaaaagctctcaaacagtgagtcggtccagcagcagatggagttcttgaacaggcagctgttggttcttggggaggtcaacgagctatatttggaacaactgcagaacaagcactcagataccacaaaggaagtagaaatgatgaaagctgcctatcggaaagagctagaaaaaaacagaagccatgttctccagcagactcagaggcttgatacctcccaaaaacggattttggaactggaatctcacctggccaagaaagaccaccttcttttggaacagaagaaatatctagaggatgtcaaactccaggcaagaggacagctgcaggccgcagagagcaggtatgaggctcagaaaaggataacccaggtgtttgaattggagatcttagatttatatggcaggttggagaaagatggcctcctgaaaaaacttgaagaagaaaaagcagaagcagctgaagcagcagaagaaaggcttgactgttgtaacgacgggtgctcagattccatggtagggcacaatgaagaggcatctggccacaacggtgagaccaagacccccaggcccagcagcacccggggcagtagtggaagcagaggaggtggaggcagcagcagcagcagcagcgagctttctaccccagagaaacccccacaccagagggcaggcccattcagcagtcggtgggagacgactatgggagaagcctctgccagcatccccaccactgtgggctcacttcccagttcaaaaagcttcctgggtatgaaggctcgagagttatttcgtaataagagcgagagccagtgtgatgaggacggcatgaccagtagcctttctgagagcctaaagacagaactgggcaaagacttgggtgtggaagccaagattcccctgaatctagatggcccgcacacacctcccccgaacccggacagtgttggacagctacatatcatggactacaatgagactcatcatgaacacagc

SEQ ID NO: 16 - protein, 1128 aa, Danio rerio
MAKEQPSVFDLIPLLGSTDLHELEQVKTLLQETLSADKGTMLLNSLVDYFLQTNSSQAVDILSSVREPHDKYLLDKMNECMGKQSCRLSTITLLGHVVRKQPPWIHKIARFPLLVSLLKCLKTDSDVVVLITGVLVLITLMPMIPQTNKQLLCDYFDIFGRLAAWNQRHPGHAQGVFAVHLHASVYSLFHRLYGMYPCNFVSYLRAYYSMKENVDTFEEVVKPMLEHVRIHPELITGTKDYEVDPIRWKRFEPHDIVIECAKISLDSKEASSEEGYSSLADHIQRRAVHHSQSCSELSFHEISSVTPIGAICQSLSKSQPYLTVTQDSGSSAQTPNNQSDEVKSSGMKEELWSPSSICGMSTPPSSRGMSPTTVSDISLSASHLSGRIPSTPGDGKWTLSPSTPSGSSPLPSLSEEIGQAAQNTSSTQAKTCEEVKKVTSRSINNSSENGGDAKTPVSLAQLSDIVKRTEGEREDDTINEEILSLTHGKLDFGSRPGLESSFSGSLDTLLSSHPNHEQNLVSTPDRVECSAKAGEHQLPRSLWPFTPIENGKHSKCSLSDLTKESMPYEPLFDLALPKVASLFLERKTAEAFRRVEGERDHQDEMEGEDLSVTSPLEVLDRVIQQGHDTHGKVLKRTFSSPSQSSDPHLKGLVPAVSDELDMLRSQLLLLHNQLLYERHKREQHALRNRRLFGRIVNTTALEEQNNSMKTQLKLQEVEIQAVKVSLKEEQRRSVHIQEDREAVVNQLQTQIQQLQKERNDYYSKMQVLKSELQENQTKAGKMEAELQKANNKVCNMGHMLSQLSIKLNNSENMEQQMAFLNKQLLLLGEANKLYMEEIDRLGPEAHKELNMLQASSVREGERLRQSSLQQSQRLEAAQQRITDLENQLTKKEQVIREQKKLLDNVKRQATEQLQASENRYLAQKNITQALQTELLELYSKIELKNLKTNAPAEPISDPSSPTNQRPNGNSAGPSAPSLPADLMKREEGAHVCVNGEISAASPQTPTALINGSQEEDLSALTHSFPSLPCQPFAVGSYPSTRSFLGKKAREMFRNKSESHYDGDSPTLTCLSKDLKVEPITDPTESLESEEAADQFDLQPMDRPRAYNVQSRRHQDLRIMDYNETHHVH

SEQ ID NO: 17 - DNA, 3384 bp, Danio rerio
atggccaaagagcagccaagtgtgtttgacctcatccctcttctggggtccactgacctacatgagctggaacaggtcaaaaccctgctgcaggaaactctcagtgcagataaagggaccatgttgctgaatagccttgtagactatttccttcagacaaactcatctcaagctgtggacatcctgtcttctgtccgggagcctcatgataagtatcttttagataagatgaatgaatgcatgggcaagcagagctgtcgtctctccaccatcaccctccttggtcatgttgttcgcaaacagccaccatggatccacaagatcgcacgcttccctctgcttgtctcactgctcaaatgcctcaagactgactcagatgtagtagttctcataaccggtgtgctggtgctgatcactctaatgcccatgattcctcaaaccaacaaacaacttctttgcgactactttgatatctttggacgtcttgctgcctggaaccaacgtcatccaggccatgctcagggagtgtttgcagtccatctacatgccagcgtgtactctcttttccatcgtttatatggcatgtatccttgtaatttcgtttcgtacctgcgtgcctattacagtatgaaggaaaatgttgacacgttcgaggaagtggtgaagcccatgttggaacatgttcggatacatcctgagctcatcactggaaccaaagactacgaggttgatccaattagatggaagagatttgagccacatgatattgtgattgaatgtgccaaaatttcactggattctaaagaggcatcatctgaagagggctactcctctttggcggatcacatacagcgacgcgctgtacaccatagccaatcatgttcagagctcagtttccatgaaatctcatcagtcacaccaataggtgctatttgtcagtctctctctaaatcacaaccttatctcactgtaacccaagactcaggttccagcgcacagacccccaacaatcaaagtgatgaggtcaaatcctcagggatgaaggaggagctgtggagcccttcctcgatatgtgggatgagcacccctcccagttctagaggaatgtccccaaccacagtttctgacatctcacttagtgcctcacacctctcaggacggatccccagcaccccaggggatggaaagtggactctatctccctccacaccctcggggtcatcccctctcccatcgctctcagaggaaatagggcaggctgcccaaaacaccagcagtacccaggctaagacgtgcgaagaggtgaagaaagtcacatccagaagtattaataacagcagtgagaatggaggtgatgccaagactccagtgtcgttggcacagctctctgacattgtcaaaaggacagagggggagcgagaggatgatacaatcaatgaggagattttaagtctaacacatgggaaactagactttggatctaggcctggtctggaatcctcattttctggctccttagacacccttctctcatctcatcccaaccatgagcagaatttggtatccacaccagacagggttgaatgctcggctaaagcaggagagcaccagcttcctaggtctctctggccgttcacccccattgaaaatgggaagcacagtaaatgttctctgtctgatctcacaaaagaatccatgccttatgagccgctgtttgacctcgccctgccgaaggtggcatctctgtttttggagaggaagactgctgaggcctttcggagggttgaaggagaaagagatcaccaagatgagatggaaggagaggatctttctgtcacctcccctctggaggtgttggaccgggttatccagcagggtcacgacacccatgggaaagtgctgaagagaactttctcttctccaagccaatcatcagacccacacttgaaaggtcttgtgcctgcagtgtcggatgagctagacatgttacgcagtcaattgctgctgctgcataaccagttgctttatgagagacataagagagagcaacatgcgcttcgtaaccgtcgtctgtttggccgcatcgtcaacactactgcactggaagagcaaaataactccatgaaaacgcagctcaaacttcaggaagttgagatccaggctgtaaaggtaagcctgaaagaggagcagcggcgctctgtgcacatacaggaagacagagaggctgtcgtgaaccagttacagactcaaattcagcagttacagaaggagcggaatgactactactcaaagatgcaggtgctaaagagcgagttgcaggagaatcagacaaaggctggcaaaatggaggcagaactgcagaaagccaataacaaagtgtgtaatatgggtcacatgctcagccagctatctatcaagctgaacaatagcgaaaacatggagcagcagatggctttcctgaataagcagctgctgcttttgggtgaagccaacaagttatacatggaagaaattgaccgcttgggccctgaggcgcacaaggagctgaatatgctgcaagcatcttcagtgcgggagggtgagcgtttgaggcagagctctcttcagcagagtcagagacttgaggctgcacagcagcgcatcacagacctggagaaccaactcaccaagaaagagcaagtcattcgggagcaaaagaaactgctggacaatgtcaaacgacaggccacggagcagctgcaggcttcagaaaacaggtatttggctcagaagaacatcactcaggcattacaaactgagctgcttgagctctatagcaagatagagctcaaaaacctgaaaactaatgcaccagcagagcccatttctgatcccagcagtcccaccaaccagaggccaaatggcaacagtgcaggcccatctgctccctctctaccggctgacctgatgaaaagagaggaaggggcgcatgtgtgtgtcaatggtgaaatatctgcagcgtctcctcaaacacccacagcactcattaatggcagtcaggaagaagatctttccgcccttactcattcatttccctccctcccctgccaaccctttgcagttggctcttaccccagcaccagaagctttttgggtaagaaagccagagaaatgttccgcaataagagtgagagccactatgatggcgactcgcccaccctcacctgcctctcaaaggatcttaaagttgagccaatcacagaccccacagagagtttggagtccgaggaagctgctgaccagtttgacctgcagccaatggatagaccccgggcatataatgttcagagtaggaggcatcaggatctgcggatcatggactataatgagacccaccatgtgcat

SEQ ID NO: 18 - protein, 899 aa, Schizosaccharomyces pombe
MPLQSLVKALWNVLHEEESEGYPDLTELIAEVESYQQRYPKQNPTNSQKIRHILDEIYEKTPFNNTRRRILWLAVLKTVIPLLILDRQAVGEWWDQIFFPFLNSPTQLKPVFSDLKSILFYILIFHDEDEWGGDLRRECAEETITRLVDLYVSKAIENLGDVESQEQRNQTIECLVNVLVHYGIQRPKELSSCFCHHFLNPPTRIPILSVMVEVIRRQGPRLYEIPQTGFYDLVLKCAEFDTSPILLSYALSFILMILSHICNSLDDSLYRLFCIYLRFSMIDPTSGFPSSTASGNWEVFHDFMSTYASTTTSQTDSSYNDVHDIVGSSQPDYLESLDYSQLFSILYALYPINFLEFLRDPKLYASKHNFQIRYSFNQELLSTKSDGLLGRHLAHSNFLKYTAETELTDKSRWTRLDSIAVVALCNSLNAVGIAESVMDPFGGKLPTTYEETSSATGLLAYPNESHDIASEPFSISWPQNPSISGSVHSATTFDKAQLSNTEDSYDNISHGTSYSEGVSSIHMVKGERGSNNLELTSESLSSTNDTIRRLQRDLLFLQNELRFEKFVRQQHLQNIGKLHREHILDMAVESERQKLLLTNKRYKAQIELLNSEIDKHRSESQAALNRRVKWENDFNNKIKALREEKKAWKSEESELKSSIESLISQLESIRNSQIDIAFSKNQLELKLQLYETKLKEYEQHLSCVNISKKQVSSSSDTSFGNTKMDSSMILSNSEAVSDEQERELIESEKHRMKLESENLHLQANIELLKKDLEAINVVYEAKIFDLEKRLSSEANAPELHNPVNLNYDAQLSKISEIKENYDELLTRYRELEGKFLESQAEVEELKNFQKPLVDTGSSIHSSPGLQQSKFIIRNDSLHPKVGPPRRQSTDTSRSTFRQY

SEQ ID NO: 19 - DNA, 2697 bp, Schizosaccharomyces pombe
atgccgctgcaatcgctagtcaaagcattatggaacgtccttcatgaagaagaatcggaaggctatcccgatttgactgagttaattgcggaagttgagtcgtatcagcagcgttatcccaaacaaaaccccactaactcacaaaaaataagacacatcttggacgagatctacgaaaagacgccgttcaataatacaaggcgacggatactatggctagctgtgttgaaaacggttataccgttgttgattttggatcgccaagcagttggtgaatggtgggaccagatcttttttcctttcttgaattctcctacccaattgaaaccggtatttagtgacttgaaaagtattttattttacattttaatttttcacgacgaggatgaatggggtggagacttgagacgggagtgcgctgaggaaactatcactcgtttggttgacctttacgtttctaaagctatcgaaaatctaggtgacgtggaatctcaagagcaaaggaatcaaactattgaatgcttggttaatgttctcgtccattatgggatacagagaccaaaggaactttcttcttgtttttgtcatcattttttaaatcctccaacccgaatccctattttatcagttatggttgaagtaatacggcggcaaggtcctcgtttatatgaaataccacagactggcttctatgaccttgttttaaaatgtgcggaatttgatacttcaccaatcttactgtcgtatgcattatctttcatccttatgatactgtctcacatttgtaactccttagacgattccttatatagattgttctgtatctatcttcgattttcaatgattgacccgacctctggcttcccatcgtctactgcgtctggtaattgggaggtgtttcacgattttatgtcaacgtatgcctcaactaccacctctcaaacagatagctcttacaatgatgttcatgatattgttggttcttctcaaccagattatctcgaaagtcttgactactctcaacttttctcaattttgtacgctctgtaccctataaactttttagagtttcttcgggatcctaagttgtatgcttctaaacataatttccaaatacgttattcctttaatcaagaactcctctccacaaagtcggatggtcttctcggtagacatttggcacattctaattttcttaaatatacagcagaaacagaacttaccgataaatcccgttggacaagacttgattctattgctgtagtggccctatgtaatagtcttaacgctgttggaatagccgaaagtgttatggatccttttggtggtaagcttcccaccacttatgaagagacaagttcagctactggattattggcctatccaaatgaaagccatgatattgcttctgagcctttttccatatcttggcctcaaaatccatcaatatcgggttctgttcattccgcgactacatttgacaaagcccaattatcaaatactgaagattcttatgataatatttcgcatggtacatcatatagtgaaggtgtttcctcaattcacatggttaaaggtgaacgcggctcaaataatttggaactgactagtgaaagtttgagcagcacgaacgatacaattcgacgcttacaaagggatctgctatttcttcagaatgagctccgttttgaaaaatttgttcgtcagcagcacttgcaaaatattggaaagcttcatcgtgagcacatactagatatggctgtggaatcagaaaggcaaaaattgcttttaaccaacaaaagatataaagctcagatagagttactgaattctgaaattgacaaacatcgctcagaatcacaggcagctctaaatcgtagggttaagtgggaaaatgattttaacaacaaaataaaagcgttacgagaagaaaagaaagcttggaaaagtgaagaatcagaactcaaatcatcaattgaatcattaatctcgcaattggaatcaatacgaaattctcaaattgacattgcgttttcaaaaaatcaactggagttgaagcttcaactctatgaaactaagttgaaagaatatgaacagcatttgagctgcgttaatatctccaaaaagcaagtctcttcgagctctgatacttcttttggaaatacgaagatggacagtagtatgattctctctaacagtgaagctgttagtgatgagcaagaaagagagttgatcgagtcagaaaaacatcgaatgaaactggaatcagaaaacttgcatcttcaagctaatattgagctactgaaaaaggacctggaagccattaatgttgtttacgaggctaagatttttgatttggaaaaacgactttcatctgaagcgaacgcacctgagttgcataatcctgtcaacttaaattacgacgctcagttgtctaaaatatccgaaataaaagaaaattacgatgagctattaacgcggtaccgtgagttggagggcaagtttttagaatcacaagctgaggtcgaagaattaaaaaactttcaaaaaccgcttgttgatactggaagctcaatacacagtagccctggtttgcaacaaagcaagttcataattcgtaatgactctttgcatcctaaagttggcccgccacgtagacagtctactgatacaagtcgttctacgtttcgccaatac

SEQ ID NO: 20 - protein, 1100 aa, Drosophila melanogaster
MVIEKIIGDLESNMTLENEEAKRKLVELLSQNKEQWVVKFMLDYFFTTGSQRILEVLVKAQAPHDGYIFDKLDDCLKQSQHRVQSLQVFCFIVRHHPTWLYKIEKHRLIKSVFKLMTHEKEIVPLMSALLCIITLLPIIPNSVPNFLNDLFEVFGHLASWKLQNSNKLPDEKLVHLQLGLQMLFHRLYGMYPCSFIAYLVEFIKRGNGGGIFQHTIKPLLNTVRVHPMLVTATPETEVNNTRWKEMEPHDVVMECANLSLPVLLPETSNEDGSYAYPMTPGYSRMTSNTSNTDYSYQLREFQQSRNVYTRFDSFASGDDVGPIWSPHNEIATTSSGIPLTPTTSFILPLQPAMNSQLMVGMTGSSPPEAAVEATPETTPLKDMRDIKQPGRAVNSHAVRAIFAVSQPSSPMRKDQQSQFSFPDVSREAEESSHSYLEVNRGTAYDRRLSQVIQDRHNVERSVNTPCPSSLPEINSDLSLVGGSVYPSVTQEVAAVCGECNETDRNLCSVGGLHMPTSRSMHQLAKKRRNRMASYSGNGSCADSRSSAAKKASWSTEAENPMRRTKSCSALSGMRQQHLEENDDEADCSSQRQRGENGNTQKTGSRLQRSGRNLAISAPKDTARSCTHASTQTVEGLDSAPAQYENWLIELLLECKEQRIDYERNLLYPQDILDEYIKHAIKANESFDAEQGQLMCLQLEYESYRRSIHAERNRRLMGRSRDKRSLEMERDRLREQLKNFDAKNKDLANKMDQAIRLANERQNIHQEELGEMRAKYQHELEEKKCLRQANDDLQTRLTSELARHKEMNYELESLRGQVFSLGTELQHTQQQADIGLQCKQELARLEAEFIIMGEVQVRCRDRLAEIDNFRARDEELQMLQESSNLELKDLRHSLDEKTSQLESMKHKISDLQAQLANSEKAMTEQKRLLSTVKDEYEEKFKSVNKKYDVQKKIIMQMEEKLMMMMQQPQGTTGHNTCSPDTDRTDIASSIERNSPLSTSLASSESLSASLRSTELKNLHQLVDTPTIPDVLNSMAGGAQFEDEVRPPAVDLASSASTASAINIVPHALDLPSTSGGIGHTLTHPHPHPHLHLQQQQQDQLQ

SEQ ID NO: 21 - DNA, 3300 bp, Drosophila melanogaster
atggtgattgagaagatcattggtgacctggagtccaacatgacgctggagaacgaggaggccaagcgcaagcttgtggagttgctatcccagaacaaggagcagtgggtggtaaagttcatgctggactacttctttacaactggatctcagcgcattttggaggtactggtcaaagcccaggcacctcacgatgggtacatctttgacaagctggacgactgcctaaagcagtcgcagcaccgagtgcagagcctccaggtgttctgcttcattgtgcgccatcaccctacttggctgtacaagatcgagaaacaccggctgatcaaaagtgtttttaagcttatgacgcacgagaaggagatagttccgctgatgagcgccctgttgtgcataattactctgctgccgatcataccgaattctgtgcccaactttcttaacgatctgtttgaggtgttcgggcatttggcctcgtggaagctgcagaatagcaataaactgccggacgagaagctcgtccacctgcagttgggtctacagatgctatttcaccgcctgtacggcatgtatccgtgcagctttattgcctatttagtggagttcatcaagcgaggcaacggcgggggcatcttccagcatacaatcaagccgctgttgaacactgtgcgagtgcatcccatgctggtgacggccacgccagagactgaggtaaacaatacgcgatggaaggagatggagccgcatgacgtggttatggagtgcgccaacctatcgctgcccgtcctcttgcccgagacgagcaacgaagacggcagctatgcgtatcccatgacgccaggatacagtcgcatgacttcaaatacctcgaatacggactacagctatcagctgagggagtttcagcaatcgagaaatgtctacacccgcttcgattcgtttgcctcgggtgatgatgtgggtccgatctggagtccgcataacgagattgccacgaccagtagcggcataccgcttacacccaccacatcgtttattttgccactacaaccggctatgaactctcagcttatggttggcatgactggctcctcaccgcccgaagcagctgtggaggccacaccggaaaccacccccttaaaggatatgagggatatcaagcagccgggacgtgcggtcaattcgcatgctgtgagagctatcttcgccgtaagccagccttcttcacccatgcgcaaggaccagcagagtcagttcagtttcccggatgtctctcgcgaggcggaggagagcagccactcatatctggaggtcaacagaggaactgcctatgaccgtcgcctgtcgcaggtcatccaggacaggcataacgtggagcgatctgtaaacacaccttgtccaagcagcctgccagaaattaactccgatttatcccttgttggtggttccgtctatccatctgtcacgcaggaggtcgctgcagtttgtggcgagtgcaacgagacggataggaacctctgcagtgtgggtggacttcatatgcccaccagccgatccatgcaccagctggcaaagaagcgccgcaatcgtatggcaagctacagtggaaatggttcctgtgcggacagcagaagttcggcggcaaagaaggcaagttggagtactgaggcagagaacccaatgcgacgaaccaaatcctgctcggccctttccggaatgcggcagcagcatctggaggagaatgatgacgaggccgattgttcgagccaaagacaaagaggggagaatggaaatacgcaaaagactggcagccgcctgcagaggagcggccggaacctggccatttcggcgcccaaggatacggctagaagctgcacccatgcctccacccagacggtggaaggactggacagtgctccagcgcagtacgagaattggcttattgaactcctgctggagtgcaaggagcaaagaatcgactatgaaaggaaccttctgtacccgcaagatattctagacgaatacattaagcatgcgatcaaggccaatgagtcctttgacgccgagcagggtcaactgatgtgcctacagctggaatacgaaagctaccgtcgatccattcacgcagagcgcaatcgacgactcatggggcgaagcagggacaagcgcagcctggaaatggagcgggatcggttaagggagcagcttaagaacttcgatgcgaagaacaaggatctggcaaacaaaatggatcaggccattcggttggccaacgagcgccagaacatccaccaggaggagctgggcgaaatgagggctaagtaccagcacgaactggaggaaaagaagtgcctgcggcaggcaaacgatgacctgcagacgcgactcaccagcgagttggcgcgccacaaggagatgaactatgaactggagtctctgcgaggtcaggtcttcagtttgggaaccgagcttcaacacacccagcagcaggcggacattgggctgcagtgcaagcaggagctggcacgactggaggccgagtttattatcatgggtgaggtgcaagtgcgttgccgcgaccgtctggctgagatcgataacttcagggcccgcgacgaggaactgcagatgctacaagagagcagcaacctggaactgaaggatctgaggcacagcctggacgagaagacatcacagctggaaagcatgaagcacaagatcagcgacctgcaggcccagctagccaacagcgagaaggccatgacggagcaaaagcgacttctaagcaccgtcaaggatgagtacgaagaaaagtttaagtccgtgaacaagaagtatgacgtgcaaaagaagataattatgcagatggaggagaagctgatgatgatgatgcagcagccgcaaggaacaacaggtcataacacctgttccccggacacggacagaactgacatagcttcatccattgaacgcaactcaccgctatccacgtcgctggcctcgagcgagagcttatccgccagcctacgctccacggagctgaagaacctgcaccagctagtggacacgcccactattccggatgtgctgaacagcatggccggtggcgctcagttcgaggacgaagtgcgtccgccggccgtggatctggcctcctcggcaagcaccgccagtgccatcaacatcgtgccgcacgccttggacttgccgtcgacctccggcggcatcggtcacacgctcacccacccacatccgcatccgcacctgcacctgcagcaacagcaacaggatcaactgcag

SEQ ID NO: 22 - protein, 1809 aa, Rattus norvegicus
MAKPTSKDSGLKEKFKILLGLGTSRPNPRCAEGKQTEFIITAEILRELSGECGLNNRIRMIGQICDVAKTKKLEEHAVEALWKAVSDLLQPERPPEARHAVLALLKAIVQGQGDRLGVLRALFFKVIKDYPSNEDLHERLEVFKALTDNGRHITYLEEELAEFVLQWMDVGLSSEFLLVLVNLVKFNSCYLDEYIAPMVHMICLLCIRTVSSVDIEVSLQVLDAVVCYNCLPAESLPLFIITLCRTVNVKELCEPCWKLMRNLLGTHLGHSAIYNMCRIMENRSYMEDAPLLRGAVFFVGMALWGAHRLYSLKNSPTSVLPSFYEAMTCPNEVVSYEIVLSITRLIKKYRKELQAVTWDILLDIIERLLQQLQNLDSPELRTIVHDLLTTVEELCDQNEFHGSQERYYELVESYADQRPESSLLNLITYRAQSIHPAKDGWIQNLQLLMERFFRNECRSAVRIKVLDVLSFVLLINRQFYEEELINSVVISQLSHIPEDKDHQVRKLATQLLVDLAEGCHTHHFNSLLDIIEKVMARSLSPPLELEERDLAVYSASLEDVKTAVLGLLVILQTKLYTLPASHATRVYETLISHIQLHYKHGYSLPIASSIRLQAFDFLLLLRADSLHRLGLPNKDGVVRFSPYCLCDCAELDRASEKKASGPLSPPTGPPSPVPTGPAVRLGHLPYSLLFRVLLQCLKQETDWKVLKLVLSKLPESLRYKVLIFTSPCSVDQLSSALCSMLSAPKTLERLRGTPEGFSRTDLHLAVVPVLTALISYHNYLDKTRQREMVYCLEQGLIYRCASQCVVALAICSVEMPDIIIKALPVLVVKLTHISATASMAIPLLEFLSTLARLPHLYRNFAAEQYASVFAISLPYTNPSKFNQYIVCLAHHVIAMWFIRCRLPFRKDFVPYITKGLRSNVLLSFDDTPEKDKFRARSTSLNERPKSLRIARAPKQGLNNSPPVKEFKESCAAEAFRCRSISVSEHVVRSRIQTSLTSASLGSADENSMAQADDNLKNLHLELTETCLDMMARYVFSNFTAVPKRSPVGEFLLAGGRTKTWLVGNKLVTVTTSVGTGTRSLLGLDSGDLQGGSASSSDPGTHVRQTKEAPAKLESQAGQQVSRGARDRVRSMSGGHGLRVGVLDTSAPYTPGGPASLGAQAAPAARPEKPCAGAQLPAAEKANLAAYVPLLTQGWAEILVRRPTGNTSWLMSLENPLSPFSSDINNMPLQELSNALMAAERFKEHRDTALYKSLSVPAAGTAKPPTLPRSNTVASFSSLYQPSCQGQLHRSVSWADSAVVLEEGSPGEAHVPVEPPELEDFEAALGTDRHCQRPDAYSRSSSASSQEEKSHLEELAAGGIPIERAISSEGARPTVDLSFQPSQPLSKSSSSPELQTLQDILGDLGDKTDIGRLSPEAKVRSQSGILDGEAATWSAPGEESRITVPPEGPLPSSSPRSPSGLRPRGYTISDSAPSRRGKRVERDNFKSRTAASSAEKVPGINPSFVFLQLYHSPFCGDESNKPILLPNESFERSVQLLDQIPSYDTHKIAVLYVGEGQSSSELAILSNEHGSYRYTEFLTGLGRLIELKDCQPDKVYLGGLDVCGEDGQFTYCWHDDIMQAVFHIATLMPTKDVDKHRCDKKRHLGNDFVSIIYNDSGEDFKLGTIKGQFNFVHVIITPLDYKCNLLTLQCRKDMEGLVDTSVAKIVSDRNLSFVARQMALHANMASQVHHRRSNPTDIYPSKWIARLRHIKRLRQRIREEVHYSNPSLPLMHPPAHTKVPAQAPTEATPTYETGQRKRLISSVDDFTEFV

SEQ ID NO: 23 - DNA, 5427 bp, Rattus norvegicus
atggccaaaccaactagcaaagattcaggtttgaaggagaagttcaagatactattaggactgggaacgtcaaggccaaatcctaggtgtgcagaaggcaaacagacggagtttatcatcacagcggaaatcttgagagaactgagtggtgaatgcggcctcaacaatcgaatccggatgatagggcagatctgtgatgtggcaaaaactaagaaacttgaagagcatgcagtggaggcactttggaaggctgtctcagacttgctacagccagagcggccaccggaggcccgacatgcagttctggccttgttgaaggccattgtgcagggacagggtgatcgtttgggggttctccgagccctcttcttcaaagtgatcaaggactatccttccaacgaagacctccatgaaaggctagaggtttttaaggccctcacagacaatggaaggcatattacctacttggaagaagaactggcagagtttgtcctgcagtggatggatgttggcttgtcctcagaattccttctggtactcgtgaacctggtcaagttcaacagctgctaccttgacgaatacattgcgccaatggttcacatgatttgtctgttatgtatccggacagtgtcctctgtggacatcgaggtgtccttgcaagtgctggatgctgtggtctgctacaactgcctgcctgctgagagcctgcctctgttcattatcaccctgtgccgcaccgtcaacgtcaaggagctgtgtgagccgtgctggaagctgatgcgaaaccttctaggtacccatctaggccacagtgccatttacaacatgtgccgtatcatggagaacagatcctacatggaagacgcccccttgttgaggggagccgtgttctttgttgggatggcgctctggggagctcaccgactctattcgctcaagaactcccccacatctgtgctgccgtctttttatgaggccatgacctgtcccaatgaggtggtgtcatatgagattgttctgtccataacaagactcatcaagaagtacaggaaggagctccaggctgtgacatgggacattctgctggacattattgaacgactgctccagcaactccagaacctggacagcccggaactcaggaccattgtccatgacctgctgaccactgtggaggagctgtgtgaccagaacgagttccacggatcgcaagaaagatactatgaactggtggagagctatgcagaccagaggcctgaatcctctctcttaaacctgataacctacagagcccagtctatccacccagccaaggatggctggatccagaacctgcagttgttgatggagaggttcttcaggaacgagtgccgcagtgccgtgcgcatcaaggtgctggacgtcctgtccttcgtgctcctcatcaacaggcagttctacgaggaggagctgattaactcggtggtcatctcacagctctcccacatccccgaggataaagaccatcaggttcgaaagctggctacccagctgctggtggacctggcagagggctgccacacccaccacttcaacagtctgttggacatcattgaaaaggtgatggctcgctccctttctccacccctggagctggaggaaagggatctggctgtgtactcggcctccctggaggatgtgaagacggcagtcctcgggctcctggtcatccttcagaccaagctgtataccttgcccgccagccatgccacacgagtgtatgagacactcattagccacatccagctccattacaagcatggctactccttgcccattgctagcagcatccggctgcaggcctttgacttcctgctgctactgcgggctgactcgctgcatcgactgggcctgcccaacaaggatggggtcgtgcgattcagcccttactgcctctgtgactgcgcggaactggacagggcctcagagaagaaagccagcggacccctttcaccgccaactgggccccccagccctgtgccaacgggccctgctgtgaggctcggccacctgccctactccctgcttttccgtgtcctgctgcagtgtttgaagcaggagaccgactggaaggtgctgaagctggtgctcagcaaactccctgagtcgctgcgctacaaagtcctcattttcacctccccctgcagtgttgaccagctgtcttctgctctctgctccatgctttcagccccgaagacccttgagcggctccgaggcaccccagaaggcttctccagaactgacttgcacttggctgtggtccctgtactgacagcgttaatatcttaccacaactatttggataagactagacagcgtgagatggtttactgcctagaacaaggccttatctaccgctgtgccagccagtgtgtggtagccttggccatctgcagtgtggagatgcccgacatcatcatcaaggcgctccctgtcctggtggtgaagctgacacacatctctgctacagctagcatggccatccccctcctggagttcctgtctaccttggccaggctgcctcacctctacaggaacttcgccgcagagcagtatgcaagtgtgtttgccatctccttgccctacaccaacccctccaagttcaaccagtacattgtgtgtctggcccatcacgtcatagccatgtggttcattaggtgccgactgccctttcggaaggattttgtcccttatatcactaagggtctgcgttccaatgtcctcctgtcctttgatgatacccctgagaaggacaagttcagagcacggagcaccagtcttaatgagagacccaaaagtttgaggatagccagagcccccaaacaaggcctgaataactctccacctgtgaaagaattcaaagagagctgtgcagccgaggccttccggtgccgcagcatcagtgtatctgaacatgtggtccgcagcagaatacagacatctcttaccagtgccagcttggggtctgcggatgagaactctatggcccaggctgatgacaacctgaagaatctccacttggaactcacagaaacatgtctggacatgatggcccgatatgtgttctccaacttcactgcggttcccaagaggtcccctgtgggagaattcctcctggcagggggcaggaccaaaacgtggctggttggaaataagctggtcactgtgacaacgagtgtgggaactggtacgcggtcgttgctgggcctggactccggggatctgcagggtggctcagcatcaagctctgatcctggcacacatgtgaggcagacaaaggaagcaccggccaaactggagtcccaggctgggcagcaggtgtcccgtggggcccgggaccgagtccgctccatgtcagggggccacggccttcgagttggtgtcctggacacttcagctccctacaccccaggaggccctgcttctctgggagcacaggcagcaccagcagcaaggcctgagaagccctgtgcaggagcccaacttccggcagcggagaaggcgaacctggcagcctatgtgcctttgctgacccagggctgggcagagatcctggtccgcaggcccacaggaaacaccagctggctgatgagcttggagaacccgctcagccccttttcctcagacatcaacaacatgcccctgcaagagctgtctaatgcccttatggctgctgagcgcttcaaggagcaccgggacacggccctatacaagtcattgtcagtgccagcggctggcacagccaagcctcctactctcccacgttctaacacagtggcttctttctcctccctgtaccagcccagctgccaaggacagctgcacaggagcgtttcctgggcagactcggccgtggttcttgaggagggaagtccaggagaggctcatgtgccagtggagccccccgaattggaggactttgaggcagcattaggtacagacaggcactgccagcggcctgacgcctacagcaggtcttcctcagcatctagccaggaagaaaagtcccacttggaggagctggctgcagggggtatccccattgagcgggccatctcctctgagggggctcggcctacagtggacctgtccttccaaccctcacagcctttgagcaagtctagctcttctcctgagttacagaccttacaggacatccttggagacctaggggacaagactgacattggacggctgagtcctgaggctaaggtccgatcccagtcggggatcctggatggggaagcagctacctggtcagctccgggtgaagagagccgtatcacagtcccacctgaaggtcccctgccttccagttctccccgctcccctagtggcctccggccccgaggctataccatctctgattcagctccatcacgaaggggaaagagggtagaaagggacaacttcaagagcagaactgcggcctccagtgctgaaaaggtgccaggcatcaaccctagctttgtgttcctacagctctaccattcacccttttgtggtgatgagtccaataagcccatcctgttgcccaacgagtcctttgaacggtcagtgcagctccttgaccagatcccatcctatgacactcacaagattgctgtcctgtatgtgggagaaggccagagcagcagtgagctggccatcctatcaaatgagcatggctcttataggtacacagagtttctgacaggcctgggtcggcttattgagctgaaggactgccaaccagacaaggtgtacttaggtggactggatgtgtgtggcgaggatggacagttcacgtactgctggcatgatgacatcatgcaagctgttttccacattgctaccctgatgcccaccaaggatgtggacaagcaccgttgtgacaagaaacggcacctgggcaatgactttgtttctatcatctacaatgattctggtgaggacttcaagctgggcaccattaagggccagttcaacttcgtccatgtgatcatcacaccgctggactataaatgcaacctgttgaccctgcagtgcaggaaagacatggagggccttgtggacaccagtgtggccaagattgtgtctgaccgcaacctgtcctttgtggcccgacagatggccctgcatgcaaatatggcctcacaggtacaccacaggcgatccaaccccacggacatctatccctccaagtggatcgcaagactccgccacattaagcgtctccgtcagcggatccgtgaagaagtgcactattcaaacccaagcttgcctctgatgcaccctccagcccataccaaagtcccagctcaagcccctacggaggctacacccacctacgagacaggccagcggaaacgcctcatttcctctgtggatgacttcacagagtttgtg

SEQ ID NO: 24 - protein, 1543 aa, Arthroderma otae CBS 113480
MSSDDVPQAPQNKPSTSSFADVFKTLAVGRPKSQSPVLHAQNAGGSLSPEMEGRRGSRVTFGFEALHRGSVATSASDASSTPSIDTILHNLAADQPLQGAADEAEKAVRYLHGFSGDQVIVVWEQAAYLIDHPSSPEARNAGGILLESVSGRQDLPLSGPRLTNNRGLDRPLYEQTAAIRSKLKRWRGQRPAGTTFDETVLGELFQLIVDVVTLQRNHPSPEVIQLILDQIFTVCKKTSAAVDIKNALVVFDAIIATLGVPDPSFIPLLEVLCSIHASVKSLAGPTSRAVRSLAKSGKQPEMVETLHTFLWETSERPDRNLNVTRGAVDIFRDLLVAYGQDGMPTISFERLITSLQRASLRKDGRIDTDILEVCLNALQGEFAPVSLQHDWSEFVSVMMACSERAIDATAPGSPASNISPVSLPTSTPPTKTSMADDVRSNIIANLTRIAASLETLWPKLDRAQKLEAWRLLADIHRHLSHSQSELAIRLLGAERLCHPSSSDNWISHSWKLVQDYILDRDKAPETRILALAAFRDAYFSENAPELYVKEGLLRSLTKGFADEDSFLFLQELVAFLVDVTACSDEDTLNLIVDTLSQPMNADENTEDMSPSGTNPVPHYVLASDTLAPSLSDAATLGLIRIFLKGLETKSQVVLTTYQRLINIALSTSRPSDCRLSALRLLFRLRCDTSGVIHVASSIDNGHLTASLLRTAESTAKSSVSEESSTERTANNNDPSSTPNSRSSLKDQSTTPLGAHGHRVMARRPSRWVAPIWAFDEHNKLPDMTVLKLGSNVYAFKNSCTDGEPEHDTNVILKVNLWAEAIIALLQREKDWDIYSYVSAHVSAQLANRDFFSAALPQIKLLRSVLCEQIKNESFPEPLGWTGVKKKDIAICLLEALTMLVSFHEHFAKGEQDEIVRAFMYGIGSWEGTSRGCIHALSVCCHEIPLSVTKCLNAILDKMSKVITRSHIAVHILEFLALLARLPEVYVNLRDEEIRTVFGICIRYIQTSREQRYKSTEVANGRSASMPSRVSNGSKDPVSHPPTEAIDFHGGENVARYVYHLTYHVMVFWFLSLKLQDRPNHVGWITKRLVFNDEQGKEVIEEQSQVFIDMMQRVAYSDLGDTIPFENFPPSPSDGPVVKKTWIVGTSIVTVETACASGLNQVTKRQASGTTYSIFQQRTAPVLPHQIPTNPGSYSLSDDPSTRTSVLPSHALLQMTASAFPTAAPLQPLPLPDDDFTRRAISAFDRNSIVDGHKIGVLYIGEGQTDETEIFANEHGSPDYEFFIASLGTKVSIENPKFNPQGLHFERDGKYIYAWRDRVSEIIYHVATMMPTNLEADPYCVNKKQHIGNDFVNIIFNRSGSEFDFNTISTQFNFVDIVITPASRVSSGKDKEGEFTVNDFNRRLYTVKVLSKPGMPELSAAAIPKVICGKNLAAFVRIIGLNASVFSLVCSHGGEYISSWQNRLREIRRLRDRAYACSSTDITSPDISSGSNSGSMGDGQYPASRRNTKQSVDDGSLRSGLGTERNLAMDCNAFQSLDFSRWTR

SEQ ID NO: 25 - DNA, 4629 bp, Arthroderma otae CBS 113480
atgtcttccgacgatgtcccccaagcgccacaaaacaaaccgtctacctcatcctttgccgacgtgttcaagaccctcgcggtagggcgtcccaagtcgcagtcaccggtattacatgcgcaaaacgctggaggctcgctgtcacccgagatggaaggcagaagaggcagccgagtaacctttgggttcgaggccttgcatcgaggaagcgtggccacgagtgcatctgatgcgtcttcgacccccagtatcgacacgatccttcacaatctagctgcggaccaacctctccagggtgcagccgacgaggccgagaaggcggtgcggtatctacacggtttcagtggtgaccaggtcatcgtggtctgggaacaagctgcctaccttatcgaccaccccagttctccggaggcacgaaatgcaggtggtatattgctggaaagcgtctctggccgtcaggacctgcccctgtctgggccccgtcttacgaataatcgtggcctggatcgtcctctatacgagcaaacggcggctatccgatcaaaacttaagcggtggagaggacagaggcccgccggcactacctttgatgagaccgtactgggtgagctgttccagcttattgtcgacgtcgtcactctgcaacgcaaccacccgagcccagaggtcatccaacttatcctcgatcagatattcactgtttgcaagaaaacgagtgctgccgtggacataaaaaacgcgctcgtcgtctttgacgctatcatcgcaacgttaggcgtaccggacccaagcttcatccctttactggaggttctgtgcagtatccacgcgtcggtaaagtcgttagctggtccaacatccagagccgttcgcagtctagctaagtccggaaagcagccagagatggtagagactcttcacaccttcctttgggagacttctgagcgacctgaccgtaacctgaatgttacacgcggtgctgtggatatcttccgagatctacttgtcgcatatggccaagacgggatgcctacgatctcgttcgaacgccttatcacctcccttcaacgagcatctctccgcaaggatggtcgaattgacaccgacatcctagaagtctgccttaacgcgttgcagggtgaatttgcaccagtctcactgcaacatgactggtccgagtttgttagcgtcatgatggcttgctcagaaagagccatagacgcaacagcgccaggctcaccagcttcaaacatatctcccgtctctctccctacatccacccctcccactaaaacctccatggccgatgatgtacgatctaacataattgccaaccttacccgcattgcagcctctcttgaaaccctgtggccaaagttggatagggctcagaagctcgaagcatggcgccttttggccgacattcatagacaccttagccattctcagtcggaacttgctatacgcctgttaggggccgaaagactatgccatccatcctcatctgacaattggattagtcactcgtggaaactagtgcaagattatattttggatcgtgacaaggccccggaaactcgcatccttgctttggccgctttcagagatgcatatttcagtgagaatgcaccggaattatatgttaaggaagggcttcttcgctcccttaccaagggcttcgccgatgaagattcattcttgtttttgcaagaattagtagcgtttctagttgatgtaacagcctgctctgacgaagacacgctgaacctcatagttgataccctaagccagcccatgaatgctgatgagaatacggaagacatgtccccatccggtaccaacccagttccgcactatgttttggcctcagacacgcttgcaccatccctaagtgacgcagcaacattgggccttattcggatctttttgaaagggctggaaactaaatctcaggttgttttgacaacttaccagagattaataaatatcgcactgtcaacaagccgcccttctgactgccgcctctcagcgctccgactccttttccgactccgttgtgacacctcaggtgttatccatgttgcatcatccattgataatggacatttaactgctagcttgttacgcaccgctgaatcaaccgccaagtcaagcgtcagtgaagagtcgtctactgaacgcacggcaaacaacaatgatccatcatctactccaaatagcagatcatctctgaaagatcaatcaaccacccctctaggagcccatggccatcgcgttatggctcgacgcccttctcgatgggttgcaccgatctgggcttttgatgaacataataagctacctgacatgacagtactcaaattaggcagcaatgtctacgcattcaagaactcatgcaccgatggagagcctgagcacgacactaacgtcatattgaaagttaacctatgggccgaggctatcattgctcttctccagcgtgaaaaggattgggatatctatagctatgtttctgctcatgtcagtgcacagcttgccaatagagatttctttagtgcggcattgccccagatcaagttgctgcgaagcgttctgtgcgagcagatcaagaacgagagtttccccgaacctcttggctggacaggagtaaagaaaaaggatatcgcaatctgcctcctcgaagctctgacgatgcttgttagcttccatgagcacttcgcaaagggtgaacaagatgagatcgtccgtgcgttcatgtacggaattggttcatgggagggcacgtctcgaggatgtatccatgcactcagcgtttgctgtcacgaaatccctctctcggtgacgaagtgtttgaatgctatccttgataaaatgtccaaagtcatcactcgatctcatattgccgttcacattctcgaattcctcgctctgcttgcacgcttgcctgaagtgtatgtcaatcttcgtgatgaggagatcaggactgtttttggaatttgtattcggtatattcagacgtcaagggagcaacgatacaagagcactgaggttgccaacggccggtccgcatctatgccctcacgagtgagcaatggctcaaaagaccctgtctcacaccctcctacagaggctattgattttcacggtggagagaatgttgccagatatgtttatcatttgacgtaccatgttatggtcttttggttcctatcgcttaagttacaggatagaccaaaccacgtcggttggatcacaaagcgattggtgttcaatgatgagcaaggaaaagaagtcatcgaagagcagagccaagtattcattgatatgatgcaacgagttgcatactcagacttgggagatacaattccattcgaaaacttcccaccgtcgccgtcagatggcccagttgtaaaaaagacttggattgttggcacaagtatcgtcacggtcgaaacagcatgtgcctctgggttgaatcaagtcacgaaaagacaggcctccgggacaacgtactccatcttccaacagcgaaccgcacctgtcctgcctcatcaaatacctacaaatcctggctcatactccctttccgatgacccttcaacgcgaacctcagtcctaccaagccatgctctccttcagatgacagcctctgcatttcccacagcagccccgttgcagccactcccattacctgatgatgactttacccgtcgggcaattagcgcgttcgataggaacagtattgttgacggccataagattggtgttctgtatatcggcgaaggccagacggacgagacagaaatatttgcgaatgagcacggcagcccagactatgaattcttcattgccagccttggaaccaaggtctccatcgagaatcccaagttcaacccgcaaggcctccattttgaaagagatggcaagtatatctatgcttggcgcgatcgagtctccgagattatataccatgtcgctaccatgatgcctacgaacttggaagcggatccttattgtgtaaacaagaagcagcacattggcaacgacttcgtgaacatcatcttcaaccggtccggttccgagtttgacttcaataccatctctacccaattcaactttgtggacatcgtcattacaccagcttctcgggtaagctccggtaaagacaaagaaggagaattcaccgtcaacgacttcaaccgaagactttatactgtcaaagtgctgagcaagcccggtatgcccgaattatcggccgcggcaatcccaaaggtcatctgcggcaaaaatctcgctgcattcgtcaggatcatcggtttaaatgcatctgtcttttccctcgtctgcagtcacggcggcgaatacatttcttcctggcaaaaccgcctgagagaaatccgccgtcttcgagatcgtgcatacgcctgttcgtccaccgatattactagcccagacatctcatcgggcagcaattcaggttctatgggcgatggccaatacccagcatcacgacgcaacaccaaacagtctgttgatgacggcagtttgcgcagcggcctcggaactgaacgcaaccttgcaatggactgtaacgccttccagagcttggatttctcacgctggactcgt

SEQ ID NO: 26 - protein, 1339 aa, Schizosaccharomyces pombe
MNNKSLLDLSSEPAIKDTDILSYFDANLPFKKRHQIVRSIYHGLKYYIYSSTSIIQVWQNIQDFISTKNDNAAFRELVYNLMMRYVSTQKHLSIIERHTFFQTIEKDPFQDIAELQLRLKSLSVLTDEGHKISGIENRVGPLLSAWFNQYLQWQSQATELQGKDADSKLVHLLFFKSLFKFSTNLVKFQWFLVPEPQMLQLVNSVVQICNHARLEDVVTEVLMFFDSMIRYSVIPKASLYDTVLILCSTYISTYSYSKLAQSVIFNLISSPVSNLAFENVFNILQYNRSNVNAVRGAVRLMRFLMLQEVKNDAIASITLSSSIEFTEFPLGFNENVDFEILGTVYLFLRTPSILNRLNFLDWHRILNILMYCSQYLPLKASTSKEAFSKTAAFANIYDRVLDFLDFEALIPLLQQFQVKLVFFLKDVLPVLKPKIRKKLLRLFETYNLIFPCNQYWVFNLEFLLGIYQCKTFDLEDRALLFKLVEDACSVADENSAPILCSKFLFPVIESFSKESDDCVVSPVYNMLFFLSVNFQNPGLKDCIDHIFQQLISDTSSVTVRRLATSTLIRLFYYYYDLRDAVPIQETLAKMLEILETPSFPFVSRMLCLQFFLRFRANGTSIYICENIDLNEPFKVLNVDSELIPAVYPISDSFVNSATVEKHIWERKENDLIKISNHSTEYGKDFVTFPTSSLLRFYRKSMATESNWTILMFMITHLADQISNRSMFIGALEEIYNLLDFMCDIVFERVSISAEIPSNIRKANIMIPILQNVQMLFVYHDQFSRAQEDELVSVFFAGLQKWNEACHVSIHSLMLCCYELPVSIRKQLPAILVTLSRLITKPDLSVHILEFLCSLARLPDLIANFTDADYRQIFAIALKYIQHRDFTKESKDSNDTESILKNSYSSYVLALAYSVLQIWFLSLRLTERKKFVPWILRGLKLASEGKPLEDLCLVQYDMMQQFCYSNSDINNQTSTFVDSDVESETWIRGNSLFTINVSVNSGFLEAVIRRPTGTTQYTFRNEASLQKFLWEENLTSSKALTRGLLCTPSSFVSHFLDPHGISLYNQPLLLPSNDDSVRRAISVFDRIPVIESLKAGLVYVGYQQRREADILANTNPSEDFLTFLNGLGTLFELKTDQKVFAGGLDRENDIDGAFAYCWKDKVTQMVFHCTTMMPTNIEHDPGCTLKKRHIGNDFVTIIFNESGLEYDFDTIPSQFNFVNIVITPESESIRRTGRQIKFYKVKALTKYDIDFSLFRRYKIVSSDALPAIVRDVTLNAAVFSHIYHRSAGDYVHIWAERLRQLKRLREKFQASVLPEDYNLDEQTKTKLQNGTNFSDFTSYL

SEQ ID NO: 27 - DNA, 4017 bp, Schizosaccharomyces pombe
atgaacaataaatcacttttggatctttctagtgagcctgctatcaaagatacagacattctatcatattttgatgctaaccttccttttaaaaaaaggcatcagattgtaaggagtatttaccatggcttaaaatactatatatattcatcgacttctatcattcaagtatggcaaaatattcaggattttatctccaccaaaaacgataatgctgcttttcgggagcttgtatacaatcttatgatgagatatgtttctacacaaaagcatctctcgattatagagcgccacactttttttcaaactattgagaaagatccttttcaagatattgctgaattacagcttcggcttaagagtttgagtgttttaacggatgaagggcacaaaatctccggtattgagaatagggttggtcctttgttgagtgcatggtttaaccagtatcttcaatggcaatctcaagcaactgaattgcaaggcaaagacgcagattcaaagctcgtacatttgcttttttttaaaagcctttttaagttttctaccaacttagtaaaatttcagtggttcctagtgcctgaacctcaaatgctacagcttgttaattccgtggtacaaatatgtaatcacgctcgcttagaagatgtggttactgaggttctgatgttttttgattccatgattcgctactcggttattccaaaagcttcactgtatgatactgttcttattttgtgtagcacatacatcagcacctactcatattcgaaacttgcacaatctgttatatttaatcttattagtagtcccgtgtcaaatctggcttttgaaaacgtatttaatatacttcaatacaatcgctcgaatgttaatgctgtacggggcgctgtgcgtttaatgcgttttctcatgctgcaagaagtgaaaaatgatgcaatagcttctataactttaagttcaagtattgaatttactgagtttccattaggatttaatgagaatgttgattttgagatccttggtactgtgtatctttttttacgtacaccttctatattaaatcgccttaattttttggattggcatcggattcttaatattttaatgtattgttcacaatatctacctttgaaagctagtacatcgaaggaagcattttcgaagactgcagcatttgctaatatatatgatcgtgttttggattttttggatttcgaagccttgattccactgctgcaacaatttcaggttaagctggtattctttttgaaggatgttcttccagtgttaaagccgaaaattcgaaaaaaacttttacgtctttttgagacttacaatcttatatttccttgcaatcaatattgggtattcaaccttgaatttttacttggtatttaccaatgcaaaacatttgatcttgaagatcgagctcttttatttaagcttgtggaagatgcttgctctgttgctgatgaaaattcggcccccattttgtgttcgaaattcctttttccagtaattgaaagcttttctaaggaatctgatgattgtgttgtttctcctgtatataatatgttgtttttcttgtctgtcaattttcaaaacccgggacttaaggattgtatcgatcatatttttcaacagctaatttccgacacgagctctgttactgttcgcagacttgccacgagtacattaattcgtcttttttattattattatgacttaagggatgctgtgcctattcaagaaacactggctaaaatgttagaaatcttggagactccatctttcccttttgtttctcgaatgttgtgtttacaattctttcttcgatttcgagcaaatgggacgtctatttatatatgtgagaatatagatttgaatgaacccttcaaagtcttaaacgttgattcagaactgattcctgcagtttatcccatttctgattcttttgttaactcagcaacagtagaaaagcatatttgggaacgaaaagaaaacgatttgattaaaatttcaaatcattctacagaatatggcaaggactttgtaacgttcccaacgtcttccttgttaaggttttatcgaaaatccatggctacagagtctaactggactatccttatgttcatgattacccaccttgctgatcaaataagtaataggagtatgtttatcggcgcattagaagaaatctataatttacttgattttatgtgtgatatcgtttttgaaagagtttctatatctgctgaaattccttctaatattcgtaaggccaacatcatgataccaatcttgcaaaacgtacaaatgctttttgtttatcatgatcaattcagcagggctcaggaagatgaattagtcagcgttttttttgcagggttgcaaaaatggaacgaagcatgtcatgtttctattcattcacttatgttatgctgttatgaattacccgtttcaattcgaaagcagttacctgcaattttagtcactttgtcaaggttaatcaccaaacctgatttgagcgttcacattttagaatttttatgcagtttagcaagattacctgatcttattgcaaactttacggatgctgactatagacaaatttttgcaattgctttgaaatacattcagcacagagatttcactaaggagtcgaaagattcaaatgatacggagagcatattaaaaaactcttactcatcatacgttctagctctagcctatagcgtgttacaaatatggtttctttcgttaagattaactgagcgtaaaaaattcgtaccttggatattgcgtggactgaagcttgcttctgaaggtaaaccattggaagatttatgtcttgttcaatatgacatgatgcaacaattttgctattctaattccgacatcaataaccaaacatctacttttgttgactcagacgtggaaagtgaaacttggattcgtggtaattcactctttacgataaacgtgtcggtaaactctggattcttagaagctgtcattcgtcgcccgacaggtactacgcagtatacttttcgaaatgaagcaagtctgcaaaagtttttatgggaggaaaatctcacaagttcaaaggctcttacgagggggcttttgtgtactcctagcagctttgtctctcatttcttggatccacatggtatttctttatacaatcagcctcttctgcttccctctaatgatgattctgtgcgtagggctataagtgtttttgaccgtattcctgtaattgaatcactgaaggctggtcttgtgtacgttgggtaccaacagcgtcgagaagcagacatattggcaaatacaaatccttctgaagatttccttacttttcttaacggtctgggcacattattcgaattaaaaacagatcaaaaagttttcgctggtggtttagatagggaaaatgatatagacggtgcgtttgcgtattgttggaaagataaagttacccaaatggtgttccattgcactacaatgatgccaactaatatagaacatgaccctgggtgcacattaaagaagcgtcatattggaaatgactttgttactattatattcaacgaatctggtttagagtacgactttgatacaattcctagtcagttcaactttgtgaacattgttattacccctgaatccgaatctatcagaagaacagggcgtcaaattaaattttataaagttaaagcgttgaccaaatatgatattgactttagcttattccgtcgttacaaaattgtttcaagcgatgcattaccggcaattgttcgtgatgtaactctgaacgctgccgttttttcacatatatatcatcgcagtgctggtgattatgttcatatatgggctgaaagattacgacagttaaaaagattacgcgaaaaatttcaagcttcagttttaccagaagactacaatctcgacgagcaaacaaagaccaagcttcaaaatggcacgaatttttcggactttactagttattta

SEQ ID NO: 28 - protein, 1515 aa, Verticillium albo-atrum
MSRSPGDVASSPDSSRAGGLANVFKGWTGARLTKSRPPIAQSIFPPRPLNKLKNGSPNERAAAADSLRYAISEYPLSPVLDIWYAAKDLIDAGKASAARIAGWELLAECVKHKSSTDLERKEFFQTLTAPANPEDFHLQLASLVDLTNHGRDLSGFDYDVIPLLRKWLREAYQAVKVARKHASRDAKRNPKLRVSVAGEEKNLQQLFTFIVEVTKFSSNVADETALAGLVNALVAICLNTIVEDDLRACIGVIDAIVTFGSLPSGSFRECVLVLGSIFCLVPNLHKPAWHTLSIILKSHNGHATVRVLLDLLRDLPADGNSKGKDTRDIRGALAVLEKLVAKSTEKGYPTVPFTLLMNGLVATVQCTPSARVHCAVLKLLNSLFSDSDGKLHHMIAEENWTTALSIATECSKKATQATSGDNDATKTETESDRPYEAIRRELLRMIARLESLMTSKNGSFVPRPVVIQFFMDVHTLIPDSTAVVVLDYFQEFRCCSPSDLEWEDNLALVRRGFFGNRQRSSHVRLLALRSITDAYEMMDLVGEEMEEDCIATLVKDLLSDVSAETDVAVLDALVSLMVDVAIASDQELFNFITSTLKSLVSSDRLEPPASQALSPGAPPTLERSPFESESSKSDIVTQGFVRLFMRCMNANGLKSAKLFDILVSIARGSHNQIDARLTAMKLLFRLRADWASSIFLTVDTEAEGLAASLYRTEASLARKQAGDATLSARASRAETWRPCEICQSYIIQSCRHPGSIEPKAEEDSMHHVALNVTAWLSTINTIFENGCDWEVFSMILVHLPSQLSNHAMFRGALTEMQRLRRVVCEQIRLNAFQEPPTYTGLRRADVAICLFHSLTMILSYHEHFLKDQEDEIVRAFVHGISTWESKCLVQMLQKMAQIITQPHVAMHILEFLAGLSRLPALYVNFREEEYRIVFGICIRYLQYIRDKQQNHRALELSPTTVDGNSASTDDLPQYVYSLAHHVITFWFLALRLPDRSNHVGWIAKNLFANVESGQGLEEQAMVTIDFMQRVAYADANESSCDPLFTKERFGEILTRRWLIGNSIVTIEQATATGWAQITKRQPSGTSSYVVRENFRPPPAHQTMQGTEVVRASDHSNNVLPSHLMVQLMTSIPQTSESLRPIPLPDDDAVQRAVRVFDRNSTVDGHKVGVIYIGEGQTDEVEILSNVSGSSDYVEFLNGLGTLTKLKGSTFNTQGLDRDYDSDGQYTFCWRDRVTEMVFHVITQMPTNLERDPQCILKKRHIGNDFVNIIFNDSGHPFRFDTFPSEFNYVNIVITPESRASFIASREAPPADKQQFMPFYKVQLMSKPGFPTISPASETKMVSLKALPGFIRLLALNASVFSLVWANREGGDTVSSWRNRLREIKRLREKHGYTPATPTNHQITFGQSPFGPPQAGGSTPSPPTTSLGGGHHGGSLAAATHSQAAADLSRPASSVRDSFGSSLRRSSVATFFTSTSEQTSHRSSMLSTTTTTNDTEISPSTGLDSLVESVDFSRWA

SEQ ID NO: 29 - DNA, 4545 bp, Verticillium albo-atrum
atgtcccgctcgccaggagatgtcgcatcctcgccagactcgagccgcgccggaggcctcgccaacgtcttcaagggctggacgggggccaggttgaccaagtcccggcctcccatcgcccagtccatcttccctcccaggcctctgaacaagctcaagaatggttcgccgaatgagcgtgccgccgcagccgattcgcttcgctatgccatctcggaatacccactcagccccgtcctggatatctggtacgctgcgaaagacctgatcgatgcaggaaaggcaagtgcggctcgtatcgcaggttgggaactgctcgccgaatgtgtcaagcacaaatcatccaccgacctcgagcggaaagagttcttccagacccttacggcgcccgccaacccagaggactttcatctccagctcgcatcacttgttgacctgacgaaccacggccgcgacctctctggattcgactatgacgtgattccgctacttcgaaaatggctgcgcgaggcgtaccaagctgttaaggtggccaggaagcacgcgagccgtgacgccaagagaaacccaaagttgagagtctctgtagcgggcgaagagaagaacctacagcaactctttacgttcatcgtcgaagtcaccaaattcagttccaacgtcgctgacgagactgcgctcgccggcttggtcaacgctcttgtcgccatatgcctgaacacgatcgttgaagacgatctgcgtgcgtgcattggcgtcatcgatgccatcgtaaccttcggatctttgcccagcggcagcttcagggaatgcgtcttggttctgggttccatcttctgcctagtgcccaatctccacaagcctgcttggcacactttgtcgatcattctcaagtcgcacaacggacacgccacagtgagggtactgttggaccttcttcgcgacctgccagcggacggtaacagcaagggcaaggacactagagacattcgcggcgccctggctgtcctagaaaagctcgtggcgaagagtactgagaaggggtaccccacagtccccttcaccctcctcatgaacggtctcgtggctacagttcagtgcacgccgtcagcccgggttcactgtgccgttctcaagctcctcaactcgctcttcagcgacagtgacggcaagcttcaccacatgatcgctgaggagaactggacgacggcactcagcattgccacagagtgttccaagaaagcaactcaggccacttcaggagacaacgacgccaccaagactgagactgagtcggaccgaccttatgaagcaattcgtcgtgaattgctgcggatgattgctcgtcttgaaagtctaatgacgagcaaaaatggcagcttcgtccctcgacccgtcgtcatacagtttttcatggacgtgcacactctgattcctgactccactgccgtcgttgtgcttgactacttccaggaattcagatgttgctctccatcagatctggagtgggaagacaatctggctttggtccgtcgaggcttctttggcaatcggcagcgctcttcgcatgtccgactcctggcccttcgttccatcacggatgcatacgagatgatggacctcgttggcgaggagatggaagaggactgcattgccactctagtgaaggatcttcttagcgacgtctctgccgagacagatgttgccgttttagacgcactcgtttcgctcatggtcgatgtcgccattgcgtctgaccaggagctcttcaacttcattaccagcactctcaagagccttgtttccagcgacagactggagccgcccgcctcgcaagcactttcgcctggagctccgccgaccctggagcgatctccgtttgagtcagaatcgtcaaagtcggatattgtcacccaaggtttcgtccggctcttcatgcgctgcatgaatgcgaacggactaaaatcagcaaagctcttcgatatcttggtcagcatcgctcgagggagccacaaccaaatcgacgctcggctcacagcaatgaagctcctcttcagactcagagctgactgggccagctccatcttcctaacagtcgatacggaagcagaaggcctcgcagcctctctataccgtactgaagcatcgcttgctcgaaagcaggctggtgacgccaccttgtcagcgcgagcgtctagggccgaaacatggcggccttgtgagatctgccagagctatatcattcagtcatgccggcacccaggatcgatcgaacccaaggccgaagaggacagcatgcaccatgtggctctcaacgtgacagcatggttgagtaccatcaacaccattttcgagaacggctgtgactgggaggttttcagtatgatactggtgcacctgccctcacagctcagcaatcatgccatgttccgcggggcactgaccgagatgcaaaggcttcgccgtgtcgtgtgcgagcagattcgattgaatgcttttcaggagccccccacatacacgggtctccgacgggctgatgttgccatctgtctattccacagcttgaccatgattctgagctaccatgaacacttccttaaagaccaagaagacgaaatcgtgcgagcctttgtgcacggtatttcaacatgggagagtaagtgtttggtgcagatgctccaaaagatggcccagatcatcacgcaaccacacgtcgccatgcacattctcgagttcctggccggtctcagtcggctgccagccttgtatgtcaacttcagggaggaagagtatcgcatcgtgttcggaatctgcattcggtacctgcagtacatccgcgacaaacagcaaaatcaccgagcccttgagctgtcaccaaccactgttgatggtaactctgcatcaacggacgacctgcctcagtacgtgtactcgctagcacaccatgtcatcactttctggttcctggcgttgaggctcccggatcgttccaatcacgtgggttggatcgcgaagaacctcttcgcaaacgtcgagagcgggcaaggccttgaagagcaagccatggttacgattgatttcatgcagcgggttgcgtatgcggacgcaaacgagtcatcatgcgaccctctcttcaccaaggagagattcggagagatattgacgcgaaggtggctgataggaaatagcatcgtcaccattgagcaggccacagccaccggttgggcccagattactaagaggcagccctcaggcacgtcgtcttatgtcgtcagggagaacttccgaccgccacccgctcaccagacaatgcaaggaaccgaagtggttcgggccagcgaccacagtaacaacgtgctgcccagtcatctgatggtgcaactcatgacatccatcccacagacttccgagtcgctacggcccattccgttacccgacgatgacgctgtacagagagctgtccgagttttcgaccggaactcaacggttgacggccacaaggtgggtgtgatttacattggcgagggccagacagatgaagtcgagattctctccaacgtgtctggcagcagcgactatgttgaattcttgaacggactggggaccctcaccaagctgaagggatcaacattcaacacccagggccttgaccgtgattacgattcggacggacagtacacgttctgctggcgggatcgcgtcacggagatggtctttcatgtcatcacacagatgccgacgaacctcgaacgtgatccgcaatgcattctcaagaagcgtcacattggaaacgactttgtcaacatcattttcaacgattcaggccaccctttccgctttgacacgttcccgagcgaattcaactacgttaacatagtcattacgcccgagtctcgcgcctctttcatcgcgagtcgtgaggcgccgcctgcggataagcagcaattcatgccgttttacaaggtccagctgatgagcaagcctggattcccgaccatttctcccgcgtcggagaccaagatggtatccctgaaggcgttgccaggcttcattcgcctcctcgcactcaacgcgtccgtattctccctggtctgggccaatcgtgaaggtggtgacacagtcagctcgtggcgaaatcgcctccgcgagatcaagcgccttcgagaaaagcatggctacacaccggctacgccgacgaaccaccaaatcacattcggacagtcgccctttggaccgccacaagccggcggctccacgccctcgccgccaaccacatctctcggcggagggcaccacggtggcagcctcgcggccgccacccactcccaggctgctgccgatctctccagaccggccagcagcgtgcgagacagtttcggcagcagcctgcgccgatcctctgtcgcgaccttcttcacaagcacgagcgagcagacatcgcatcgcagctccatgctatcaacgacgacaacgacaaacgacacggagatttcgccaagcaccggcctcgactctctcgtcgagtcggtcgatttctcccggtgggct

SEQ ID NO: 30 - protein, 22 aa, Artificial Sequence
  3flag tag polypeptide sequence
DYKDHDGDYKDHDIDYKDDDDK

SEQ ID NO: 31 - DNA, 66 bp, Artificial Sequence
  3flag tag nucleotide sequence
gactacaaagaccatgacggtgattataaagatcatgatatcgattacaaggatgacgatgacaag

SEQ ID NO: 32 - protein, 8 aa, Artificial Sequence
  flag tag peptide sequence
DYKDDDDK

SEQ ID NO: 33 - DNA, 24 bp, Artificial Sequence
  flag tag nucleotide sequence
gattacaaggatgacgatgacaag

SEQ ID NO: 34 - protein, 10 aa, Artificial Sequence
  myc tag peptide sequence
EQKLISEEDL

SEQ ID NO: 35 - DNA, 30 bp, Artificial Sequence
  myc tag nucleotide sequence
gaacaaaaacttatttctgaagaagatctg

SEQ ID NO: 36 - protein, 9 aa, Artificial Sequence
  HA tag peptide sequence
YPYDVPDYA

SEQ ID NO: 37 - DNA, 27 bp, Artificial Sequence
  HA tag nucleotide sequence
tacccatacgacgtcccagactacgct

SEQ ID NO: 38 - protein, 6 aa, Artificial Sequence
  AU1 tag peptide sequence
DTYRYI

SEQ ID NO: 39 - DNA, 18 bp, Artificial Sequence
  AU1 tag nucleotide sequence
gatacctatcgctatatt

(19) United States
(12) Patent Application Publication
(10) Pub. No.: US 2012/0252877 A1
(43) Pub. Date: Oct. 4, 2012

(54) METHODS AND COMPOSITIONS FOR TREATMENT OF TUBEROUS SCLEROSIS COMPLEX

(75) Inventor: Loon-Tzian Lo, St Louis, MO (US)

(73) Assignee: Theramind Research, LLC, St. Louis, MO (US)

(21) Appl. No.: 13/390,107

(22) PCT Filed: Aug. 16, 2010
(86) PCT No.: PCT/US10/45662

§ 371 Date: Apr. 11, 2012

Related U.S. Application Data
(60) Provisional application No. 61/233,832, filed on Aug. 14, 2009.

(51) Int. Cl. A61K 48/00; C12N 5/10; A61P 25/00; C12N 15/861

(52) U.S. Cl. 514/44 R; 435/320.1; 435/366

(57) ABSTRACT

The present disclosure relates to a method of treating Tuberous Sclerosis Complex (TSC). The present disclosure further provides an isolated polynucleotide molecule comprising, a polynucleotide comprising an AAV9 vector; a polynucleotide encoding a TSC1 or a TSC2, or variant thereof; and a promoter. The present disclosure further provides a pharmaceutical composition comprising an isolated polynucleotide molecule.

### CROSS-REFERENCE TO RELATED APPLICATIONS

This application claims priority from U.S. Provisional Application Ser. No. 61/233,832 filed on Aug. 14, 2009, which is incorporated herein by reference in its entirety.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present invention generally relates to methods and compositions for the treatment of tuberous sclerosis complex and related diseases or disorders.

### BACKGROUND

Tuberous sclerosis complex (TSC) is a dominantly-inherited genetic disease in which affected individuals develop growths called hamartomas, primarily in the central nervous system (CNS), kidneys and skin (Au et al., 2008). CNS symptoms in humans include learning disabilities, seizures and autism. Seizures are the most common TSC phenotype; many patients with TSC have intractable seizures which do not respond well to anti-convulsants (Napolioni et al., 2009). Treatment of TSC-related seizures often includes surgical removal of hamartomas. Drug therapy for TSC is currently in the developmental stage (see U.S. Pat. Nos. 7,416,724 and 7,169,594; reviewed in Sampson, 2009); and no treatment is currently available to address the underlying causes of various TSC symptoms.

TSC1 (chromosome 9, U.S. Pat. Nos. 6,548,258 and 6,326,483) and TSC2 (chromosome 16, U.S. Pat. Nos. 6,232,452 and 6,207,374) (Povey et al., 1994) are responsible for phenotypes in approximately 80% of TSC patients (Au et al., 2008). TSC1 encodes hamartin, a 130 kD protein without significant sequence homology to known mammalian proteins (van Slegtenhorst et al., 1997), but which contains a predicted coiled-coil protein interaction domain (van Slegtenhorst et al., 1998). TSC2 encodes tuberin (van Slegtenhorst et al., 1998), which is predicted to interact with, and be stabilized by, hamartin (Nellist et al., 1999). TSC2 is a 180-200 kD protein comprising a coiled-coil domain and a C-terminal GTPase activating protein (GAP) homology domain (Wienecke et al., 1995). Overexpression of either TSC1 or TSC2 has growth-suppressing effects (Miloloza et al., 2000; Jin et al., 1996). Mouse Tsc1+/− and Tsc2+/− models have increased numbers of astrocytes (Uhlmann et al, 2002).

The CNS phenotypes seen in TSC patients include cortical tubers, subependymal nodules (SENs), and subependymal giant cell astrocytomas (SEGAs) (Holmes et al., 2007). Cortical tubers are pathognomonic of TSC, and may be epileptogenic (Napolini et al., 2009). Histopathological studies of tubers have indicated disorganized, hamartomatous regions of cortex with abnormal cell morphology; dysplastic neurons; cytomegaly; heterotropic neurons; aberrant dendritic formations and axonal projections; and astrocytic proliferation (Holmes et al., 2007). Tubers and SEGAs can be heterogenous within the same brain.

Relevant mouse models for TSC, including conventional and conditional knockout mice, are valuable tools for studying mechanisms underlying and developing therapeutic specific for TSC. Mice with heterozygous TSC1 or TSC2 deletion survive to adulthood, and appear to have minimum neurological phenotypes; while homozygous TSC1−/− or TSC2−/− mice die in mid-gestation. With conditional knockout technology, it has been possible to generate mice with a selective neuronal TSC1 knockout. These neuronal TSC1−/− mice have a median survival of 35 days, but have limited use as an animal model for the study of epilepsy. Conditional astrocyte TSC1−/− knockout mice (“Tsc1GFAPCKO”) are viable until 3-4 months of age (Uhlmann et al., 2002), with progressive epileptic activities around 3-4 weeks of age (Erbayat-Altay, Zeng, Xu, Gutmann, & Wong, 2007).

Recently, adeno-associated virus 9 (AAV9, see e.g., U.S. Pat. No. 7,198,951) has been reported to cross the blood-brain barrier in mice (Faust et al., 2009). These authors reported that injection of AAV9 carrying a green fluorescent protein (GFP) expression vector into either a neonatal or adult mouse vein produced GFP-positive cells in the brain. In injected adult mice, greater than 64% of astroctyes were GFP-positive, and greater than 90% of the GFP-positive cells were astrocytes; demonstrating that AAV9 can efficiently and specifically deliver cargo nucleic acids to astrocytes. Faust et al. postulate that AAV9 infects astroctyes by attaching to astrocytic perivascular endfeet.

### SUMMARY OF THE INVENTION

The present disclosure is based, at least in part, on the finding that TSC1 or TSC2 delivered via an adeno-associated virus can treat Tuberous Sclerosis Complex (TSC).

One aspect of the present disclosure provides a method of treating Tuberous Sclerosis Complex (TSC). In various embodiments, the method can comprise administering to a subject in need thereof a therapeutically effective amount of a composition comprising an adeno-associated virus (AAV) vector. The AAV vector can comprise at least one polynucleotide encoding a TSC1 or a TSC2, or variant thereof. The TSC1 or TSC2 can be expressed in a plurality of cells of the subject.

In some embodiments, the AAV vector comprises a polynucleotide encoding a TSC1, or a variant thereof. In some embodiments, the TSC1 sequence can be selected from SEQ ID NO: 2, SEQ ID NO: 6, SEQ ID NO: 14, SEQ ID NO: 16, SEQ ID NO: 18, SEQ ID NO: 20, or SEQ ID NO: 22. In other embodiments, the TSC1 sequence can be a nucleic acid sequence having at least about 90% identity to SEQ ID NO: 2, SEQ ID NO: 6, SEQ ID NO: 14, SEQ ID NO: 16, SEQ ID NO: 18, SEQ ID NO: 20, or SEQ ID NO: 22. In other embodiments, the TSC 1 sequence can be a nucleic acid sequence encoding a polypeptide having hamartin activity. In other embodiments, the TSC1 sequence can be a nucleic acid sequence encoding a polypeptide of SEQ ID NO: 1, SEQ ID NO: 5, SEQ ID NO: 13, SEQ ID NO: 15, SEQ ID NO: 17, SEQ ID NO: 19, or SEQ ID NO: 21. In other embodiments, the TSC1 sequence can be a nucleic acid sequence encoding a polypeptide having at least about 90% identity to SEQ ID NO: 1, SEQ ID NO: 5, SEQ ID NO: 13, SEQ ID NO: 15, SEQ ID NO: 17, SEQ ID NO: 19, or SEQ ID NO: 21 and having hamartin activity.

In some embodiments, the AAV vector comprises a polynucleotide encoding a TSC2, or variant thereof. In some embodiments, the TSC2 can have a nucleic acid sequence selected from SEQ ID NO: 4, SEQ ID NO: 8, SEQ ID NO: 24, SEQ ID NO: 26, SEQ ID NO: 28, or SEQ ID NO: 30. In other embodiments, the TSC2 can have a nucleic acid sequence having at least about 90% identity to SEQ ID NO: 4, SEQ ID NO: 8, SEQ ID NO: 24, SEQ ID NO: 26, SEQ ID NO: 28, or SEQ ID NO: 30. In other embodiments, the TSC2 can encode a polypeptide having tuberin activity. In other embodiments, the TSC2 can be a nucleic acid sequence encoding a polypeptide of SEQ ID NO: 3, SEQ ID NO: 7, SEQ ID NO: 23, SEQ ID NO: 25, SEQ ID NO: 27, or SEQ ID NO: 29. In other aspects, the TSC2 can be nucleic acid sequence encoding a polypeptide having at least about 90% identity to SEQ ID NO: 3, SEQ ID NO: 7, SEQ ID NO: 23, SEQ ID NO: 25, SEQ ID NO: 27, or SEQ ID NO: 29 and having tuberin activity.

In some embodiments, the TSC1 or TSC2 polynucleotide is operably linked to a heterologous promoter. In some embodiments, the heterologous promoter can be a glial fibrillary acidic protein (GFAP) promoter, a synapsin-1 (SYN) promoter, a Ca²⁺/calmodulin-dependent protein kinase II (CAMKII) promoter, a myelin basic protein (MBP) promoter, a nectin promoter, a myosin light polypeptide 2 (Myl-2) promoter, a SM22α gene promoter, a human cytomegalovirus immediate-early gene (CMV) promoter, or a human ubiquitin 6 (U6) promoter. In some embodiments, the heterologous promoter is a glial fibrillary acidic protein (GFAP) promoter.

In some embodiments, the administration of the AAV vector is intravenous administration.

In some embodiments, the subject in need of treatment displays at least one symptom selected from the group consisting of: brain tubers, brain tumors, subependymal nodules, subependymal giant cell astrocytomas, vascular stromas, peripheral nervous system tumors, retinal hamartomas, seizures, mental retardation, learning disabilities, behavior problems, autism, autism spectrum disorders, attention deficit hyperactivity disorder, and sleep disturbances.

In other embodiments, the subject in need of treatment displays at least one symptom selected from the group consisting of: renal lesions caused by angiomyolipomas, simple cysts, polycystic kidney disease, renal-cell carcinoma, renal lymphangiomyomatosis, cardiac lesion caused by cardiac rhabdomyomas, dermatological lesions caused by hyperpigmented maculars, angiofibromas, fibrous plaques, papules, Shagreen patches, gingival fibromas, and pulmonary lesions caused by lymphangiomyomatosis.

In some embodiments, AAV vector is an AAV9 vector.

In some embodiments, the method of treating TSC can further include measuring any of the expression of TSC1 or TSC2, wherein at least one of the polynucleotides for TSC1 or TSC2 is comprised by the AAV vector; the activity level of a polypeptide encoded in the AAV vector; or the level of mTOR signaling in cells comprised by the subject.

In some embodiments, the method can optionally further include comparing the expression of TSC1 or TSC2 in the subject being treated for TSC to the expression of TSC1 or TSC2 in a subject who is not in need of treatment for TSC.

In some embodiments, the method can optionally further include comparing the activity level of a polypeptide encoded in the AAV vector or the activity level of mTOR signaling in cells comprised by the subject being treated for TSC to the activity level of a polypeptide encoded in the AAV vector or the activity level of mTOR signaling in a subject who is not in need of treatment for TSC.

The present disclosure also provides an isolated polynucleotide molecule comprising a polynucleotide comprising an AAV9 vector; a polynucleotide encoding a TSC1 or a TSC2, or variant thereof; and a promoter. In some embodiments, the promoter is operably linked to the polynucleotide encoding a TSC1 or a TSC2, or variant thereof. In some embodiments, the polynucleotide encoding TSC1 or TSC2, or variant thereof, is selected from SEQ ID NO: 2, SEQ ID NO: 6, SEQ ID NO: 14, SEQ ID NO: 16, SEQ ID NO: 18, SEQ ID NO: 20, or SEQ ID NO: 22. In some embodiments, the polynucleotide encoding TSC1 or TSC2 is a nucleic acid sequence having at least about 90% identity to SEQ ID NO: 2, SEQ ID NO: 6, SEQ ID NO: 14, SEQ ID NO: 16, SEQ ID NO: 18, SEQ ID NO: 20, or SEQ ID NO: 22. In some embodiments, the polynucleotide encoding TSC1 or TSC2 encodes a polypeptide having hamartin activity. In some embodiments, the polynucleotide encoding TSC1 or TSC2 is a nucleic acid sequence encoding a polypeptide of SEQ ID NO: 1, SEQ ID NO: 5, SEQ ID NO: 13, SEQ ID NO: 15, SEQ ID NO: 17, SEQ ID NO: 19, or SEQ ID NO: 21. In some embodiments, the polynucleotide encoding TSC1 or TSC2 is a nucleic acid sequence encoding a polypeptide having at least about 90% identity to SEQ ID NO: 1, SEQ ID NO: 5, SEQ ID NO: 13, SEQ ID NO: 15, SEQ ID NO: 17, SEQ ID NO: 19, or SEQ ID NO: 21. In some embodiments, the polynucleotide encoding TSC1 or TSC2 encodes a protein having hamartin activity. In some embodiments, the polynucleotide encoding TSC1 or TSC2 is SEQ ID NO: 4, SEQ ID NO: 8, SEQ ID NO: 24, SEQ ID NO: 26, SEQ ID NO: 28, or SEQ ID NO: 30. In some embodiments, the polynucleotide encoding TSC1 or TSC2 is a nucleic acid sequence having at least about 90% identity to SEQ ID NO: 4, SEQ ID NO: 8, SEQ ID NO: 24, SEQ ID NO: 26, SEQ ID NO: 28, or SEQ ID NO: 30. In some embodiments, the polynucleotide encoding TSC1 or TSC2 encodes a polypeptide having tuberin activity. In some embodiments, the polynucleotide encoding TSC1 or TSC2 is a nucleic acid sequence encoding a polypeptide of SEQ ID NO: 3, SEQ ID NO: 7, SEQ ID NO: 23, SEQ ID NO: 25, SEQ ID NO: 27, or SEQ ID NO: 29. In some embodiments, the polynucleotide encoding TSC1 or TSC2 is a nucleic acid sequence encoding a polypeptide having at least about 90% identity to SEQ ID NO: 3, SEQ ID NO: 7, SEQ ID NO: 23, SEQ ID NO: 25, SEQ ID NO: 27, or SEQ ID NO: 29. In some embodiments, the polynucleotide encoding TSC1 or TSC2 encodes a protein and having tuberin activity.

In some embodiments, the isolated polynucleotide molecule comprises a human cytomegalovirus promoter. In some embodiments, the human cytomegalovirus promoter comprises a nucleic acid sequence of SEQ ID NO: 9, or 90% identity thereto and having cytomegalovirus promoter activity.

In some embodiments, the sequence of the isolated polynucleotide molecule comprising an AAV9 vector; a polynucleotide encoding a TSC1 or a TSC2, or variant thereof; and a promoter comprises SEQ ID NO: 12.

In some embodiments, the present disclosure provides a pharmaceutical composition includes an isolated polynucleotide molecule including SEQ ID NO: 12 and a pharmaceutically acceptable carrier or excipient.

In some embodiments, the disclosure provides a virion comprising an isolated polynucleotide including SEQ ID NO:12. In some aspects, the virion is capable of delivering cargo to human cells. In some aspects, the virion comprises AAV9 capsid or an AAV9 capsid protein comprising mutations not found in naturally-occurring isolates of AAV9.

In some embodiments, the present disclosure provides a cell comprising the isolated polynucleotide comprising an AAV9 vector; a polynucleotide encoding a TSC1 or a TSC2, or variant thereof; and a promoter.

The present disclosure also provides for the use of an isolated polynucleotide molecule comprising a polynucleotide comprising an AAV9 vector; a polynucleotide encoding a TSC1 or a TSC2, or variant thereof; and a promoter. In some embodiments, the promoter can be operably linked to the polynucleotide encoding a TSC1 or a TSC2, or variant thereof. In some embodiments, the isolated polynucleotide molecule can be used the treatment of Tuberous Sclerosis Complex (TSC).

In some aspects, the polynucleotide encoding TSC1 or TSC2, or variant thereof can be SEQ ID NO: 2, SEQ ID NO: 6, SEQ ID NO: 14, SEQ ID NO: 16, SEQ ID NO: 18, SEQ ID NO: 20, or SEQ ID NO: 22. In some embodiments, the polynucleotide encoding TSC1 or TSC2, or variant thereof can be a nucleic acid sequence having at least about 90% identity to SEQ ID NO: 2, SEQ ID NO: 6, SEQ ID NO: 14, SEQ ID NO: 16, SEQ ID NO: 18, SEQ ID NO: 20, or SEQ ID NO: 22 and encoding a polypeptide having hamartin activity. In some embodiments, the polynucleotide encoding TSC1 or TSC2, or variant thereof can be a nucleic acid sequence encoding a polypeptide of SEQ ID NO: 1, SEQ ID NO: 5, SEQ ID NO: 13, SEQ ID NO: 15, SEQ ID NO: 17, SEQ ID NO: 19, or SEQ ID NO: 21. In some embodiments, the polynucleotide encoding TSC1 or TSC2, or variant thereof can be a nucleic acid sequence encoding a polypeptide having at least about 90% identity to SEQ ID NO: 1, SEQ ID NO: 5, SEQ ID NO: 13, SEQ ID NO: 15, SEQ ID NO: 17, SEQ ID NO: 19, or SEQ ID NO: 21 and encoding a polypeptide having hamartin activity. In some embodiments, the polynucleotide encoding TSC1 or TSC2, or variant thereof can be SEQ ID NO: 4, SEQ ID NO: 8, SEQ ID NO: 24, SEQ ID NO: 26, SEQ ID NO: 28, or SEQ ID NO: 30. In some embodiments, the polynucleotide encoding TSC1 or TSC2, or variant thereof can be a nucleic acid sequence having at least about 90% identity to SEQ ID NO: 4, SEQ ID NO: 8, SEQ ID NO: 24, SEQ ID NO: 26, SEQ ID NO: 28, or SEQ ID NO: 30 and encoding a polypeptide having tuberin activity. In some embodiments, the polynucleotide encoding TSC1 or TSC2, or variant thereof can be a nucleic acid sequence encoding a polypeptide of SEQ ID NO: 3, SEQ ID NO: 7, SEQ ID NO: 23, SEQ ID NO: 25, SEQ ID NO: 27, or SEQ ID NO: 29. In some embodiments, the polynucleotide encoding TSC1 or TSC2, or variant thereof can be a nucleic acid sequence encoding a polypeptide having at least about 90% identity to SEQ ID NO: 3, SEQ ID NO: 7, SEQ ID NO: 23, SEQ ID NO: 25, SEQ ID NO: 27, or SEQ ID NO: 29 and encoding a polypeptide having tuberin activity.

In some embodiments, the promoter is a human cytomegalovirus promoter.

In some embodiments, the human cytomegalovirus promoter comprises a nucleic acid sequence of SEQ ID NO: 9, or 90% identity thereto and having cytomegalovirus promoter activity.

In some embodiments, the sequence of the isolated polynucleotide comprises SEQ ID NO: 12.

The present disclosure also provides for the use of a polynucleotide comprising an AAV9 vector; a polynucleotide encoding a TSC1 or a TSC2, or variant thereof; and a promoter; in the manufacture of a medicament for the treatment of Tuberous Sclerosis Complex (TSC). In some embodiments, the promoter is operably linked to the polynucleotide encoding a TSC1 or a TSC2, or variant thereof.

In some embodiments, the polynucleotide encoding TSC1 or TSC2, or variant thereof includes SEQ ID NO: 2, SEQ ID NO: 6, SEQ ID NO: 14, SEQ ID NO: 16, SEQ ID NO: 18, SEQ ID NO: 20, or SEQ ID NO: 22. In some embodiments, the polynucleotide encoding TSC1 or TSC2, or variant thereof includes a nucleic acid sequence having at least about 90% identity to SEQ ID NO: 2, SEQ ID NO: 6, SEQ ID NO: 14, SEQ ID NO: 16, SEQ ID NO: 18, SEQ ID NO: 20, or SEQ ID NO: 22 and encoding a polypeptide having hamartin activity. In some embodiments, the polynucleotide encoding TSC1 or TSC2, or variant thereof includes a nucleic acid sequence encoding a polypeptide of SEQ ID NO: 1, SEQ ID NO: 5, SEQ ID NO: 13, SEQ ID NO: 15, SEQ ID NO: 17, SEQ ID NO: 19, or SEQ ID NO: 21. In some embodiments, the polynucleotide encoding TSC1 or TSC2, or variant thereof includes a nucleic acid sequence encoding a polypeptide having at least about 90% identity to SEQ ID NO: 1, SEQ ID NO: 5, SEQ ID NO: 13, SEQ ID NO: 15, SEQ ID NO: 17, SEQ ID NO: 19, or SEQ ID NO: 21 and encoding a polypeptide having hamartin activity. In some embodiments, the polynucleotide encoding TSC1 or TSC2, or variant thereof includes SEQ ID NO: 4, SEQ ID NO: 8, SEQ ID NO: 24, SEQ ID NO: 26, SEQ ID NO: 28, or SEQ ID NO: 30. In some embodiments, the polynucleotide encoding TSC1 or TSC2, or variant thereof includes a nucleic acid sequence having at least about 90% identity to SEQ ID NO: 4, SEQ ID NO: 8, SEQ ID NO: 24, SEQ ID NO: 26, SEQ ID NO: 28, or SEQ ID NO: 30 and encoding a polypeptide having tuberin activity. In some embodiments, the polynucleotide encoding TSC1 or TSC2, or variant thereof includes a nucleic acid sequence encoding a polypeptide of SEQ ID NO: 3, SEQ ID NO: 7, SEQ ID NO: 23, SEQ ID NO: 25, SEQ ID NO: 27, or SEQ ID NO: 29. In some embodiments, the polynucleotide encoding TSC1 or TSC2, or variant thereof includes a nucleic acid sequence encoding a polypeptide having at least about 90% identity to SEQ ID NO: 3, SEQ ID NO: 7, SEQ ID NO: 23, SEQ ID NO: 25, SEQ ID NO: 27, or SEQ ID NO: 29 and encoding a polypeptide having tuberin activity.

In some embodiments, the promoter is a human cytomegalovirus promoter.

In some embodiments, the human cytomegalovirus promoter comprises a nucleic acid sequence of SEQ ID NO: 9, or 90% identity thereto and having cytomegalovirus promoter activity.

In some embodiments, the sequence of the isolated polynucleotide comprises SEQ ID NO: 12.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a map of the vector pAAV.CMV.hTSC1.V5.RBG (“AAV9-hTSC1-V5”).

### SEQUENCE LISTING

SEQ ID NO: 1: Polypeptide sequence of human TSC1

SEQ ID NO: 2: Polynucleotide sequence of human TSC1

SEQ ID NO: 3: Polypeptide sequence of human TSC2

SEQ ID NO: 4: Polynucleotide sequence of human TSC2

SEQ ID NO: 5: Polypeptide sequence of mouse TSC1

SEQ ID NO: 6: Polynucleotide sequence of mouse TSC1

SEQ ID NO: 7: Polypeptide sequence of mouse TSC2

SEQ ID NO: 8: Polynucleotide sequence of mouse TSC2

SEQ ID NO: 9: Polynucleotide sequence of the CMV promoter

SEQ ID NO: 10: Polypeptide sequence of V5 tag

SEQ ID NO: 11: Polynucleotide sequence of V5 tag

SEQ ID NO: 12: Polynucleotide sequence of AAV9-hTSC1-V5 vector

SEQ ID NO: 13: Polypeptide sequence of TSC1 protein from Rattus norvegicus

SEQ ID NO: 14: Polynucleotide sequence of TSC1 protein from Rattus norvegicus

SEQ ID NO: 15: Polypeptide sequence of TSC1 protein from Pongo abelii

SEQ ID NO: 16: Polynucleotide sequence of TSC1 protein from Pongo abelii

SEQ ID NO: 17: Polypeptide sequence of TSC1 protein from Danio rerio

SEQ ID NO: 18: Polynucleotide sequence of TSC1 protein from Danio rerio

SEQ ID NO: 19: Polypeptide sequence of TSC1 protein from S. pombe

SEQ ID NO: 20: Polynucleotide sequence of TSC1 protein from S. pombe

SEQ ID NO: 21: Polypeptide sequence of TSC1 protein from Drosophila melanogaster

SEQ ID NO: 22: Polynucleotide sequence of TSC1 protein from Drosophila melanogaster

SEQ ID NO: 23: Polypeptide sequence of TSC2 protein from Rattus norvegicus

SEQ ID NO: 24: Polynucleotide sequence of TSC2 protein from Rattus norvegicus

SEQ ID NO: 25: Polypeptide sequence of TSC2 protein from Arthroderma otae CBS113480

SEQ ID NO: 26: Polynucleotide sequence of TSC2 protein from Arthroderma otae CBS113480

SEQ ID NO: 27: Polypeptide sequence of TSC2 protein from S. pombe

SEQ ID NO: 28: Polynucleotide sequence of TSC2 protein from S. pombe

SEQ ID NO: 29: Polypeptide sequence of TSC2 protein from Verticillium albo-atrum VaMs.102

SEQ ID NO: 30: Polynucleotide sequence of TSC2 protein from Verticillium albo-atrum VaMs.102

SEQ ID NO: 31: Polypeptide sequence of 3flag tag

SEQ ID NO: 32: Polynucleotide sequence of 3flag tag

SEQ ID NO: 33: Polypeptide sequence of flag tag

SEQ ID NO: 34: Polynucleotide sequence of flag tag

SEQ ID NO: 35: Polypeptide sequence of myc tag

SEQ ID NO: 36: Polynucleotide sequence of myc tag

SEQ ID NO: 37: Polypeptide sequence of HA tag

SEQ ID NO: 38: Polynucleotide sequence of HA tag

SEQ ID NO: 39: Polypeptide sequence of AU1 tag

SEQ ID NO: 40: Polynucleotide sequence of AU1 tag

### DETAILED DESCRIPTION OF THE INVENTION

The present disclosure is based, at least in part, on the finding that TSC1 (encoding hamartin) or TSC2 (encoding tuberin) delivered via adeno-associated virus 9 (AAV9) can effectively treat Tuberous Sclerosis Complex (TSC). Hamartin and tuberin function as a heterodimer that interacts with and sequesters an activator of mTOR (which controls protein synthesis and cell growth) and may also interact with p27 to repress cell cycle progression. Mutations of TSC1 or TSC2 can result in increased activation of the mTOR signaling pathway, a loss of control of cell growth and cell division, and a predisposition to forming tumors. A vector that can cross the blood-brain barrier, such as an adeno-associated virus (AAV) vector, encoding TSC1 or TSC2 nucleic acids, or variants thereof, can efficiently and specifically deliver TSC1 or TSC2 to CNS tissue or cells. Subsequent expression of hamartin or tuberin in CNS cells, for example in astrocytes, can decrease levels of mTOR signal pathway over-activation (e.g., by sequestering an activator of mTOR). Decreased levels of mTOR over-activation can ameliorate symptoms associated with TSC. Thus is provided a method of treating TSC where, in some embodiments, a composition including an AAV vector encoding TSC1 or TSC2, or variants thereof, is administered to a subject in need thereof.

Compositions

One aspect provides compositions that can express TSC1 or TSC2 upon administration to a subject. Such composition can include a vector, such as a viral vector, encoding a hamartin polypeptide or tuberin polypeptide, or both a hamartrin polypeptide and a tuberin polypeptide, or variants thereof having hamartin or tuberin activity, or both, respectively, or a promoter.

Vector

Various compositions described herein can include a vector for transmission of TSC1 or TSC2, or variants thereof, to target tissues or cells. A vector of the composition can include a promoter, such as a promoter operably linked to a TSC1 or TSC2, or variants thereof. A vector of the composition can be a vector that can penetrate the blood-brain barrier. For example, in compositions for treatment of a CNS phenotype of TSC, a blood-brain barrier permeant vector can provide for delivery of TSC1 or TSC2, or variants thereof, to tissues or cells of the CNS. A vector of the composition can be a vector that can not necessarily penetrate the blood-brain barrier. For example, a non-blood-brain barrier permeant vector can be used for delivery of TSC1 or TSC2, or variants thereof, to non-CNS tissues or cells. A vector of various compositions described herein can be a retroviral vector. A vector of various compositions described herein can be a viral vector. For example, an AAV vector can be used for delivery of TSC1 or TSC2, or variants thereof. A viral vector can have one or more properties or abilities such as: being non-pathogenic, have reduced or eliminated immunogenicity, have reduced or eliminated cytotoxic response, infect non-dividing cells, infect dividing cells, infect quiescent cells, have the ability to stably integrate into a host cell genome at a specific site, or have a reduced threat of random insertion or mutagenesis.

AAV

Various compositions described herein can include an AAV vector for transmission of TSC1 or TSC2, or variants thereof, to target tissue or cells. Numerous AAV vectors are known in the art (see e.g., Carter 2000 Gene Therapy: Therapeutic Mechanisms and Strategies. Marcel Dekker, Inc. pp. 41-59, ISBN 0-585-39515-2; Grieger and Samulski 2005 Advances in Biochemical Engineering/biotechnology 99, 119-145; Carter 2005 Human Gene Therapy 16 (5), 541-50). The AAV genome comprises inverted terminal repeats (ITRs) at both ends of the DNA strand, and two open reading frames (ORFs), rep and cap. The rep ORF contains four overlapping genes encoding Rep proteins required for the AAV life cycle, and the cap ORF contains overlapping nucleotide sequences of capsid proteins, VP1, VP2 and VP3, which interact together to form a capsid of an icosahedral symmetry. AAV vectors have been used for in vivo or ex vivo treatment of conditions such as cystic fibrosis, hemophilia, Parkinson's disease, Canavan disease, muscular dystrophy, late infantile neuronal ceroid lipofuscinosis, and prostate cancer.

An AAV vector can have one or more properties or abilities such as: non-pathogenic, have reduced or eliminated immunogenicity, have reduced or eliminated cytotoxic response, infect non-dividing cells, infect dividing cells, infect quiescent cells, have the ability to stably integrate into a host cell genome at a specific site, or have a reduced threat of random insertion or mutagenesis.

An AAV vector can have eliminated rep and cap from the AAV. The target gene, e.g., TSC1 or TSC2, or variants thereof, can replace all of, a substantial portion of, or a portion of the virus's 4.8 kilobase genome. The target gene, e.g., TSC1 or TSC2, or variants thereof, together with a promoter to drive transcription of the target gene can be inserted between the inverted terminal repeats (ITR) that aid in concatamer formation in the nucleus after the single-stranded vector DNA is converted by host cell DNA polymerase complexes into double-stranded DNA.

For example, an AAV vector can include ITRs in cis next to the target gene, e.g., TSC1 or TSC2, or variants thereof, and structural (cap) and packaging (rep) genes can be delivered in trans. As another example, an AAV vector can include ITRs and a cis-acting Rep-dependent element (CARE) (a portion of the coding sequence of the rep gene) in cis next to the target gene, e.g., TSC 1 or TSC2, or variants thereof, and structural (cap) and packaging (rep or the balance of rep) genes can be delivered in trans. CARE can augment the replication and encapsidation when present in cis. Introns of promoters p5 or p19 of the AAV genome can be spliced out or included in the AAV vector (forming, for example, Rep78, Rep68, Rep52 and Rep40 mRNAs). One or more introns of the cap gene encoding capsid proteins VP1, VP2, or VP3 (translated from one mRNA) can be spliced out or included in the AAV vector. One or more AUG start codons (or an upstream ACG surrounded by an optimal Kozak sequence) of VP1, VP2, or VP3 can be cut out to reduce overall synthesis level of the corresponding protein. For example, VP2 expression can be reduced or eliminated in the AAV vector.

Also useful with the present compositions are AAV capsids which comprise mosaic combinations of capsid proteins from multiple AAV serotypes, such as from AAV1, AAV2, AAV3, AAV4, AAV5, AAV6, AAV7, AAV8, AAV9, AAV10, or AAV11. As a further example, the AAV can comprise an AAV capsid protein encoded by a sequence not found in a naturally-occurring isolate of AAV. In some cases, the capsid protein encoded by a sequence not found in a naturally-occurring isolate of AAV can be an AAV9 capsid protein.

An AAV vector can form episomal concatamers in the host cell nucleus. In non-dividing cells, these concatamers can remain intact for the life of the host cell. In dividing cells, AAV DNA can be lost through cell division, since the episomal DNA is not replicated along with the host cell DNA. The AAV ITRs of two genomes can anneal to form head to tail concatamers, almost doubling the capacity of the vector. Insertion of splice sites can allow for the removal of the ITRs from the transcript.

An AAV vector can be of a serotype that can target unique cell types. For example, the AAV vector can be a serotype that can cross the blood-brain barrier. Examples of AAV serotypes include, but are not limited to, AAV1, AAV2, AAV3, AAV4, AAV5, AAV6, AAV7, AAV8, AAV9, AAV10, or AAV11. For example, an AAV1 vector or an AAV2 vector can be used to target delivery of cargo to neurons, smooth muscle cells and other non-CNS targets (see e.g., Daya and Berns, 2008). As another example, an AAV2 vector can be used as a strongly neuron specific agent for delivery of TSC1 or TSC2, or variants thereof, to target tissue or cells. As another example, an AAV9 vector can specifically target astrocytes in the CNS (see e.g., Foust, 2009).

An example of an AAV vector encoding TSC1 is AAV9-hTSC1-V5 vector (SEQ ID NO: 12).

TSC1

As described herein, TSC1, or a variant thereof, can be administered directly or via an encoding vector so as to effect expression of a hamartin polypeptide in a target tissue or cells. A composition can encode a TSC1 polynucleotide, or variant thereof, so as to provide for expression of a polypeptide having hamartin activity. A polypeptide having hamartin activity can be a polypeptide that has at least one of the following: (1) the polypeptide binds to a tuberin (TSC2) polypeptide; or (2) the polypeptide stabilizes a tuberin (TSC2) polypeptide.

A TSC1 polynucleotide can be a human TSC1 polynucleotide. For example, a human TSC1 polynucleotide can have a nucleic acid sequence of SEQ ID NO: 2. A TSC1 polynucleotide can be a mammalian TSC1 polynucleotide. A TSC1 polynucleotide can be a mouse TSC1 polynucleotide. For example, a mouse TSC1 polynucleotide can have a nucleic acid sequence of SEQ ID NO: 6. A TSC1 polynucleotide can be a rat TSC1 polynucleotide. For example, a rat TSC1 polynucleotide can have a nucleic acid sequence of SEQ ID NO: 14. A TSC1 polynucleotide can be a Pongo abelii TSC1 polynucleotide. For example, a Pongo abelii TSC1 polynucleotide can have a nucleic acid sequence of SEQ ID NO: 16. A TSC1 polynucleotide can be a Danio rerio TSC1 polynucleotide. For example, a Danio rerio TSC1 polynucleotide can have a nucleic acid sequence of SEQ ID NO: 18. A TSC1 polynucleotide can be a S. pombe TSC1 polynucleotide. For example, a S. pombe TSC1 polynucleotide can have a nucleic acid sequence of SEQ ID NO: 20. A TSC1 polynucleotide can be a Drosophila melanogaster TSC1 polynucleotide. For example, a Drosophila melanogaster TSC1 polynucleotide can have a nucleic acid sequence of SEQ ID NO: 22.

A TSC1 polynucleotide can be a variant TSC1 polynucleotide. A TSC1 polynucleotide can be a variant TSC1 polynucleotide. A TSC1 polynucleotide can be a variant mammalian TSC1 polynucleotide. A TSC1 polynucleotide can be a variant mouse TSC1 polynucleotide. A variant TSC1 polynucleotide can have a nucleic acid sequence having at least about 70% identity to SEQ ID NO: 2, SEQ ID NO: 6, SEQ ID NO: 14, SEQ ID NO: 16, SEQ ID NO: 18, SEQ ID NO: 20, or SEQ ID NO: 22 and encoding a polypeptide having hamartin activity. For example, a variant TSC1 polynucleotide can have a nucleic acid sequence having at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, SEQ ID NO: 6, SEQ ID NO: 14, SEQ ID NO: 16, SEQ ID NO: 18, SEQ ID NO: 20, or SEQ ID NO: 22 and encoding a polypeptide having hamartin activity.

A TSC1 polynucleotide can encode a TSC1 polypeptide (i.e., hamartin). For example, TSC1 polynucleotide can encode a polypeptide having a sequence of SEQ ID NO: 1, SEQ ID NO: 5, SEQ ID NO: 13, SEQ ID NO: 15, SEQ ID NO: 17, SEQ ID NO: 19, or SEQ ID NO: 21. A TSC1 polynucleotide can encode a variant TSC1 polypeptide (i.e., a variant hamartin). For example, TSC1 polynucleotide can encode a polypeptide having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, SEQ ID NO: 5, SEQ ID NO: 13, SEQ ID NO: 15, SEQ ID NO: 17, SEQ ID NO: 19, or SEQ ID NO: 21 and having hamartin activity.

TSC2

As described herein, TSC2, or a variant thereof, can be administered directly or via an encoding vector so as to effect expression of a hamartin polypeptide in a target tissue or cells. A composition can encode a TSC2 polynucleotide, or variant thereof, so as to provide for expression of a polypeptide having tuberin activity. A polypeptide having tuberin activity can be a polypeptide that has at least one of the following properties: (1) the polypeptide binds to a hamartin (TSC1) polypeptide; (2) the polypeptide interacts with a Ras-related small G-protein; (3) the polypeptide inhibits rheb activity; or (4) the polypeptide, along with a polypeptide having hamartin activity, forms a GTPase activating protein (GAP) complex.

A TSC2 polynucleotide can be a human TSC2 polynucleotide. For example, a human TSC2 polynucleotide can have a nucleic acid sequence of SEQ ID NO: 4. A TSC2 polynucleotide can be a mammalian TSC2 polynucleotide. A TSC2 polynucleotide can be a mouse TSC2 polynucleotide. For example, a mouse TSC2 polynucleotide can have a nucleic acid sequence of SEQ ID NO: 8. A TSC2 polynucleotide can be a rat TSC2 polynucleotide. For example, a rat TSC2 polynucleotide can have a nucleic acid sequence of SEQ ID NO: 24. A TSC2 polynucleotide can be a Arthroderma otae CBS 113480 TSC2 polynucleotide. For example, a Arthroderma otae CBS 113480 TSC2 polynucleotide can have a nucleic acid sequence of SEQ ID NO: 26. A TSC2 polynucleotide can be a S. pombe TSC2 polynucleotide. For example, a S. pombe TSC2 polynucleotide can have a nucleic acid sequence of SEQ ID NO: 28. A TSC2 polynucleotide can be a Verticillium albo-atrum VaMs.102 TSC2 polynucleotide. For example, a Verticillium albo-atrum VaMs.102 TSC2 polynucleotide can have a nucleic acid sequence of SEQ ID NO: 30.

A TSC2 polynucleotide can be a variant TSC2 polynucleotide. A TSC2 polynucleotide can be a variant mammalian TSC2 polynucleotide. A TSC2 polynucleotide can be a variant mouse TSC2 polynucleotide. A variant TSC2 polynucleotide can have a nucleic acid sequence having at least about 70% identity to SEQ ID NO: 4, SEQ ID NO: 8, SEQ ID NO: 24, SEQ ID NO: 26, SEQ ID NO: 28, or SEQ ID NO: 30 and encoding a polypeptide having tuberin activity. For example, a variant TSC2 polynucleotide can have a nucleic acid sequence having at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 4, SEQ ID NO: 8, SEQ ID NO: 24, SEQ ID NO: 26, SEQ ID NO: 28, or SEQ ID NO: 30 and encoding a polypeptide having tuberin activity.

A TSC2 polynucleotide can encode a TSC2 polypeptide (i.e., tuberin). For example, TSC2 polynucleotide can encode a polypeptide having a sequence of SEQ ID NO: 3, SEQ ID NO: 7, SEQ ID NO: 23, SEQ ID NO: 25, SEQ ID NO: 27, or SEQ ID NO: 29. A TSC2 polynucleotide can encode a variant TSC2 polypeptide (i.e., a variant tuberin). For example, TSC2 polynucleotide can encode a polypeptide having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 3, SEQ ID NO: 7, SEQ ID NO: 23, SEQ ID NO: 25, SEQ ID NO: 27, or SEQ ID NO: 29 and having hamartin activity.

Promoter

A composition can comprise a heterologous promoter operably linked to a polynucleotide. A heterologous promoter can be a glial fibrillary acidic protein (GFAP) promoter, a synapsin-1 (SYN) promoter, a Ca²⁺/calmodulin-dependent protein kinase II (CAMKII) promoter, a myelin basic protein (MBP) promoter, a nectin promoter, a myosin light polypeptide 2 (Myl-2) promoter, a SM22a gene promoter, a human cytomegalovirus immediate-early gene (CMV) promoter, or a human ubiquitin 6 (U6) promoter. As an example, a human CMV promoter can have a nucleic acid sequence of SEQ ID NO: 9.

A heterologous promoter can drive expression of a polynucleotide in extra-central nervous system (CNS) tissues. A myosin light polypeptide 2 (Myl-2) promoter can drive expression of a polypeptide in the myocardium. A human cytomegalovirus immediate-early gene (CMV) promoter or human ubiquitin 6 (U6) promoter can drive expression of a polynucleotide in any cell type.

A heterologous promoter can drive expression of a polynucleotide in CNS tissues. A human synapsin-1 (SYN) or Ca²⁺/calmodulin-dependent protein kinase II (CAMKII) promoter can drive expression of a polynucleotide in neurons. A Glial Fibrillary Acidic Protein (GFAP) promoter can drive expression of a polynucleotide in astroctyes. A human nectin promoter can drive expression of a polynucleotide in brain progenitor cells. A human cytomegalovirus immediate-early gene (CMV) promoter or human ubiquitin 6 (U6) promoter can drive expression of a polynucleotide in any cell type.

Formulation

Compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, preferably in purified form, together with a suitable amount of carrier so as to provide the form for proper administration to the subject.

The formulation should suit the mode of administration. The compositions of use with the current invention can be formulated by known methods for administration to a subject using several routes which include, but are not limited to, parenteral, pulmonary, oral, topical, intradermal, intramuscular, intraperitoneal, intravenous, intracranial, intraventricular, intraspinal, intrathecal, intrauterous (including into a fetus), intratumor, subcutaneous, intranasal, epidural, ophthalmic, buccal, and rectal. The individual compositions may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Compositions described herein can also be used in combination with other therapeutic modalities, as described further below. Thus, in addition to the therapies described herein, one may also provide to the subject other therapies known to be efficacious for treatment of TSC.

Method of Treatment

One aspect provides a method of treatment of TSC. Such method can include administering a composition described herein to a subject in need thereof. For example, a therapeutically effective amount of TSC1 or TSC2 protein, or of nucleic acids encoding such proteins, can be administered to a subject in need thereof so as to ameliorate one or more symptoms associated with TSC.

Treatment of TSC can include monitoring the status of TSC in a subject after administration of one or more of the compositions described herein. Treatment of TSC can comprise administering subsequent doses of one or more of the compositions described herein, at the same or different dosage or frequency, and can include subsequent additional monitoring or administration steps.

Monitoring the status of TSC in a subject after administration of one or more of the compositions described herein can include one or more of: (i) monitoring the expression of one or more of the polynucleotides comprised by an AAV vector; (ii) monitoring the activity level of a polypeptide encoded in an AAV vector; or (iii) monitoring the level of mTOR signaling in cells comprised by the subject. Monitoring the activity level of a polypeptide encoded in an AAV vector can comprise a comparison of the activity level of the protein encoded by the AAV vector in the subject, with the activity level of the same protein in a wild-type subject. Monitoring the level of mTOR signaling can comprise comparing the level of mTOR signaling in cells comprised by the subject with the levels of mTOR signaling in a wild-type subject. Such comparisons are within the skill of the art.

Subject In Need Thereof

Methods described herein are generally performed on a subject in need thereof. A subject in need of the therapeutic methods described herein can be diagnosed with tuberous sclerosis, or can be at risk of developing tuberous sclerosis. Diagnosis of the various conditions treatable by the methods described herein is within the skill of the art. A determination of the need for treatment will typically be assessed by a history and physical exam consistent with the disease or condition at issue.

For reference, a subject is considered to be wild-type for TSC if the subject does not exhibit symptoms of TSC. A subject is considered to be wild-type for TSC if the subject does not have a hypofunctional or non-functional mutation in TSC1 or TSC2. A subject is considered to be wild-type for TSC if the subject has a normal level of mTOR signaling. A subject not in need of treatment described herein can be wild-type for TSC.

A subject can be an animal subject, preferably a mammal, more preferably horses, cows, dogs, cats, sheep, pigs, mice, rats, monkeys, guinea pigs, and chickens. For example, the subject can be a human.

Diagnosis of TSC via symptoms.

A subject in need of the therapeutic methods described herein can be a subject who has one or more symptoms of TSC.

A subject in need of the therapeutic methods described herein can be a subject having one or more symptoms of a CNS phenotype of TSC. CNS-related symptoms of TSC include, but are not limited to, seizures, epilepsy, brain tubers, brain tumors, subependymal nodules, subependymal giant cell astrocytomas, vascular stromas, peripheral nervous system tumors, retinal hamartomas, mental retardation, learning disabilities, behavior problems, autism, autism spectrum disorders, attention deficit hyperactivity disorder, and sleep disturbances.

The severity of the CNS phenotypes of TSC can be defined by any means known. For example, seizures and epilepsy may be defined, at least in part, by one or more of how frequently seizures occur, how intense the seizures are, whether the seizures respond to pharmaceutical treatment, or what types of seizures occur. Cell proliferations, including brain tubers, tumors, subependymal nodules, subependymal giant cell astrocytomas, vascular stromas, peripheral nervous system tumors, and retinal hamartomas, can be categorized by one or more of their size, location, composition, or neuronal activity. Behavioral abnormalities, including mental retardation, learning disabilities, behavior problems, autism, autism spectrum disorders, attention deficit hyperactivity disorder, and sleep disturbances, can be diagnosed by known parameters.

A subject in need of the therapeutic methods described herein can be a subject having one or more symptoms of a non-CNS phenotype of TSC. Non-CNS-related symptoms of TSC include, but are not limited to, renal lesions caused by angiomyolipomas, simple cysts, polycystic kidney disease, renal-cell carcinoma, renal lymphangiomyomatosis, cardiac lesion caused by cardiac rhabdomyomas, dermatological lesions caused by hyperpigmented maculars, angiofibromas, fibrous plaques, papules, Shagreen patches, gingival fibromas, and pulmonary lesions caused by lymphangiomyomatosis.

A subject in need of the therapeutic methods described herein can be a subject having one or more symptoms of a either a CNS phenotype or a non-CNS phenotype of TSC. A subject in need of the therapeutic methods described herein can be a subject having one or more symptoms of both a CNS phenotype and a non-CNS phenotype of TSC.

Diagnosis of TSC via identification of mutations in TSC1 or TSC2.

A subject in need of the therapeutic methods described herein can be a subject who has a mutation in TSC1 and/or TSC2. Specifically, the mutation in TSC1 and/or TSC2 can be a loss-of-function or a hypofunctional mutation. The mutation can be in only one allele of either TSC1 or TSC2.

A subject in need of the therapeutic methods described herein can be a subject who expresses insufficient TSC1 or TSC2 protein, such as reduced expression levels compared to wild type. Such low expression may be caused by misregulation of the induction of the expression of either or both of TSC1 or TSC2. Such low expression may be caused, for example, by one or more mutation in the promoter of either or both of TSC1 or TSC2.

Composition

The composition used in a method of treating TSC can be any composition described herein. For example, the composition can include a virion. A virion can be an AAV virion, that is, can be a virion that includes at least one AAV capsid protein. In some cases, the AAV virion can be an AAV9 virion. For example, the composition can include a vector, such as an AAV vector (e.g., AAV9) encoding a TSC1 or a TSC2, or variants thereof. As another example, the composition can include a vector, such as an AAV vector (e.g., AAV9) encoding a polynucleotide which, when expressed, forms a hamartin polypeptide or a tuberin polypeptide, or a variant thereof having the specific activity.

Effective Amount

An effective amount of TSC1 or TSC2 protein, or of nucleic acids encoding such proteins described herein, can be an amount sufficient to (i) reduce or eliminate symptoms associated with TSC; (ii) reduce over-active mTOR signaling to a level equivalent or substantially equivalent to wild type; or (iii) reduce cellular proliferation to a level equivalent or substantially equivalent to wild-type; or a combination thereof. Symptoms of TSC can be as discussed herein.

An effective amount of TSC1 or TSC2 protein, or of nucleic acids encoding such proteins, can be an amount sufficient to, for example, in patients with seizures or epilepsy, reduce the frequency with which seizures occur, how intense the seizures are, whether the seizures respond to pharmaceutical treatment, or to alter what types of seizures occur. In cases where TSC phenotypes are associated with abnormal cell proliferation, including but not limited to brain tubers, tumors, subependymal nodules, subependymal giant cell astrocytomas, vascular stromas, peripheral nervous system tumors, and retinal hamartomas, an effective amount of TSC1 or TSC2 protein, or of nucleic acids encoding such proteins can change the size, location, or neuronal activity. In cases where TSC phenotypes are associated with behavioral abnormalities, including but not limited to mental retardation, learning disabilities, behavior problems, autism, autism spectrum disorders, attention deficit hyperactivity disorder, and sleep disturbances, an effective amount of TSC1 or TSC2 protein, or of nucleic acids encoding such proteins can cause improvements in behavior.

When used in the treatments described herein, a therapeutically effective amount of TSC1 or TSC2 protein, or of nucleic acids encoding such proteins can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to reduce or eliminate TSC symptoms.

Toxicity and therapeutic efficacy of compositions described herein can be determined by standard pharmaceutical procedures in cell cultures, experimental animals, and human subjects. The dose ratio between toxic and therapeutic effects is the therapeutic index. For example, standard pharmaceutical procedures can employ cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. The therapeutically effective dose of TSC1 or TSC2 protein, or of nucleic acids encoding such proteins can change over time. For example, as symptoms begin to improve, lower doses of a TSC1 or TSC2 protein, or of nucleic acids encoding such proteins, can be required as a maintenance dose. As another example, as symptoms improve, higher doses of TSC1 or TSC2 protein, or of nucleic acids encoding such proteins, can be required to reach cells requiring treatment. Furthermore, different means of administering an effective amount of TSC1 or TSC2 protein, or of nucleic acids encoding such proteins, can be used at different times during the treatment of a single subject.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration

According to the methods described herein, administration can be parenteral, pulmonary, oral, topical, intradermal, intramuscular, intraperitoneal, intravenous, intracranial, intraventricular, intraspinal, intrathecal, intrauterous (including into a fetus), intratumor, subcutaneous, intranasal, epidural, ophthalmic, buccal, or rectal administration.

Therapeutic methods described herein, including targeted expression of TSC1 or TSC2, or variants thereof, can address the molecular etiology of TSC and related genetic disorders. Administration of compositions for targeted expression of polynucleotides are known in the art (see generally, Veronique et al., 2009; Templeton 2008 Gene and Cell Therapy, CRC Press, 1120 pp., ISBN-10: 084938768X; Schaffer and Zhou 2006 Gene Therapy and Gene Delivery Systems, Springer, 285 pp., ISBN-10: 3540284044; Giacca 2010 Gene Therapy, Springer, 306 pp., ISBN-10: 8847016428; Herzog and Zolotukhin 2010 A Guide to Human Gene Therapy, World Scientific Publishing Company, 400 pp., ISBN-10: 9814280909) and can be adapted for protocols described herein. Therapeutic methods as described herein can target specific cell types, including brain cell types such as astrocytes, oligodendrocytes, radial glial cells, microglia, or neurons; muscle cells; or cells of a particular internal organ. Therapeutic methods described herein can target somatic cells. As described herein, an adeno-associated virus (AAV) can be used to provide for targeted expression of TSC1 or TSC2 polynucleotides, or variants thereof. The polynucleotides provided to cells can include genes, multiple genes, or both coding and non-coding sequences. If the gene therapy includes use of an AAV, the polynucleotide provided to a cell can comprise any polynucleotide comprised by inverted terminal repeats (ITRs), including one or more genes, and regulatory sequences. Generation of an AAV vector is described further as above.

Compositions described herein can be administered in a variety of means known to the art. The agents can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, intracranial, intraventricular, intraspinal, intrathecal, intrauterous (including into a fetus), intratumor, subcutaneous, intranasal, epidural, ophthalmic, buccal, or rectal administration.

A composition described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of compositions will be known to the skilled artisan and are within the scope of the invention.

Delivery systems may include, for example, an infusion pump which may be used to administer the agent in a manner similar to that used for delivering insulin or chemotherapy to specific organs or tumors. Typically, using such a system, the agent(s) is administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Compositions can be encapsulated and administered in a variety of carrier delivery systems. Examples of carrier delivery systems include microspheres, hydrogels, polymeric implants, smart ploymeric carriers, and liposomes (see generally, Uchegbu and Schatzlein, eds. (2006) Polymers in Drug Delivery, CRC, ISBN-10: 0849325331). Carrier-based systems for biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Administration of TSC1 or TSC2 protein, or of nucleic acids encoding such proteins can occur as a single event or over a time course of treatment. For example, the TSC1 or TSC2 protein, or nucleic acids encoding such proteins can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for tuberous sclerosis complex.

A TSC1 or TSC2 protein, or nucleic acids encoding such proteins can be administered simultaneously or sequentially with another agent, such as an antibiotic, an antiinflammatory, or another agent. For example, a TSC1 or TSC2 protein, or nucleic acids encoding such proteins can be administered simultaneously with another agent, such as an antibiotic or an antiinflammatory. Simultaneous administration can occur through administration of separate compositions, each containing one or more of a TSC1 or TSC2 protein, or nucleic acids encoding such proteins, an antibiotic, an antiinflammatory, or another agent. Simultaneous administration can occur through administration of one composition containing two or more of a TSC1 or TSC2 protein, or nucleic acids encoding such proteins, an antibiotic, an antiinflammatory, or another agent. A TSC1 or TSC2 protein, or nucleic acids encoding such proteins can be administered sequentially with an antibiotic, an antiinflammatory, or another agent. For example, a TSC1 or TSC2 protein, or nucleic acids encoding such proteins can be administered before or after administration of an antibiotic, an antiinflammatory, or another agent.

Molecular Engineering

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art. Generally, conservative substitutions can be made at any position so long as the required activity is retained.

Nucleotide and/or amino acid sequence percent (%) identity is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often, publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral vector infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Host strains developed according to the approaches described herein can be evaluated by a number of means known in the art (see e.g., Studier (2005) Protein Expr Purif. 41(1), 207-234; ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem. Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, Mo.; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinoformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Kits

Also provided are kits. Such kits can include the compositions of the present invention and, in certain embodiments, instructions for administration. Such kits can facilitate performance of the methods described herein. When supplied as a kit, the different components of the composition can be packaged in separate containers and admixed immediately before use. Components include, but are not limited to TSC1 or TSC2 protein, or nucleic acids encoding such proteins. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or other sterile components, each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

This example describes general methods and reagents.

Mice: Mice in which the Tsc1 gene has been conditionally inactivated in glial cells (Tsc1^{flox/flox}-GFAP-Cre knockout; “Tsc1GFAPCKO”) are generated as described previously (Uhlmann et al., 2002). Breeding pairs of Tsc1tm1Djk/Tsc1tm1Djk and Tg(GFAP-cre) mice are purchased from Jackson Laboratory (Bar Harbor, Me., USA), crossed to produce homozygous TSC1GFAPCKO mice (KO). Tsc1^{flox/flox} littermates of the Tsc1 GFAPCKO mice have previously been found to have no abnormal phenotype and can be used as control animals in experiments. Tsc1GFAPCKO mice produced in our breeding colony and their wild-type (WT) littermates are maintained in a 12 hour alternating light/dark cycle with food and water ad libitum, at an animal care facility.

EEG monitoring: Electroencephalographic (EEG) techniques, in which the EEG electrodes are in contact with the scalp are well established clinical tools used to monitor seizure activities in human subjects. The electrocorticogram (ECoG), in which electrodes are in contact with the brain is widely used in small animal research to monitor neural activity (del Campo et al., 2009). Because the skull does not interfere with the ECoG signal, the ECoG (often called EEG simply) is also the Gold Standard for identifying epileptic sites in the clinical setting. There are relatively few research video-EEG packages designed for rodent video-ECoG. Pinnacle Technology (Lawrence, Kans.) has developed a turn-key video-ECoG (Video-EEG) system specifically for mice and rats that uses a compact amplifier and a light/dark video camera, both of which feed data to a computer by efficient USB 2.0 (Chung et al., 2009). Each mouse is housed in cages with one to two other littermates until electrode implantation at 9 weeks old. After the bilateral epidural electrode implantation (Pinnacle Technology Inc. Lawrence, Kans.), mice are housed individually. Mice undergo continuous digital EEG and video recordings at 10 weeks of age, according to manufacture's instruction (Pinnacle Technology Inc. Lawrence, Kans.). At least one twenty-four hour epoch of continuous video-EEG data is obtained from each mouse and analyzed for interictal abnormalities and seizures.

Animal care: All animal care and procedures are performed in strict accord with the protocol approved by the Animal Studies Committee at University of Missouri-St. Louis, in accordance with the Association for Assessment of Laboratory Animal Care guidelines.

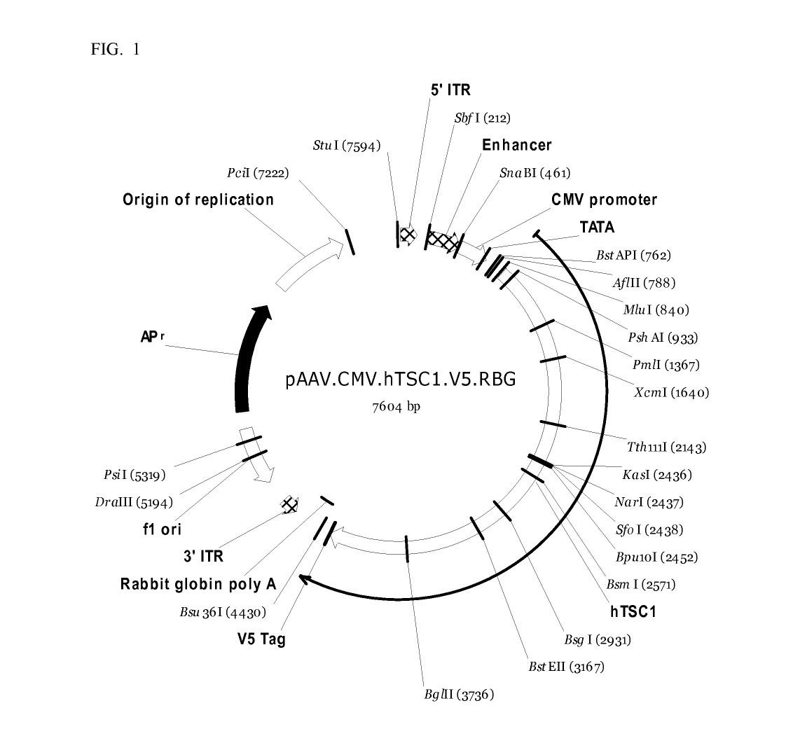

Vectors: Recombinant AAV9 vector with human TSC1 cDNA operably linked to the human cytomegalovirus (CMV) promoter and the V5 tag fused to the C-terminus of the hTSC1 gene (“AAV9-hTSC1-V5”) used in the present studies is constructed, packaged, purified, and vector titer is determined by ReGenX Bioscience at the University of Pennsylvania Vector Core (Philadelphia, Pa., USA). The AAV9-hTSC1-V5 viral particles are prepared with a titer of 1×10¹³ genome copy per ml. The AAV9-hTSC1-V5 viral particles are stored and used according to manufacturer's instructions. See FIG. 1 for a map of the vector. The polynucleotide sequence of the CMV promoter is provided in SEQ ID NO: 9. The polypeptide sequence of human TSC 1 is provided in SEQ ID NO: 1. The polynucleotide sequence of human TSC1 mRNA is provided in SEQ ID NO: 2. The polypeptide sequence of the V5 tag is provided in SEQ ID NO: 10. The polynucleotide sequence of the V5 tag is provided in SEQ ID NO:11.

Immunohistochemistry: Immunohistochemistry (“IHC”) is used herein to characterize protein expression or localization. Proteins expressed from the AAV9-hTSC1-V5 vector are assessed by immunohistochemistry with anti-V5 antibodies (monoclonal antibody R960-25, Invitrogen Inc., Carlsbad, Calif.); with anti-hTSC1 antibodies, (Cell Signaling Technology, Danvers, Mass.); and/or a Cy3-conjugated mouse monoclonal anti-glial fibrillary acidic protein (GFAP) at 1:2000 (Sigma, St Louis, Mo., USA) according to manufacturer's instructions. IHC experiments to characterize protein expression may be performed in tissue sections or on cultured cells.

IHC for V5: Chromogenic immunostaining is performed using an avidine-biotine-peroxidase complex immunostaining technique and carried out under uniform conditions. To remove any endogenous peroxidase activity, cells or tissue sections are incubated with 3% hydrogen peroxide in PBS for 10 minutes. Non-specific sites are blocked by pre-treatment with 10% normal goat serum in PBST. Cells or sections are then incubated overnight at room temperature with the monoclonal primary antibody (R960-25, Invitrogen) diluted to 1/12500 in PBST with 10% goat serum. After being washed 3 times 5 minutes with PBST, the cells or sections are incubated for 30 min at room temperature with goat anti-mouse biotinylated secondary antibody (Dako, Glostrup, Denmark) diluted at 1:300 in PBST. The cells or sections are washed in PBST 3 times 5 min and incubated with Strept-ABC-HRP complex (Dako) for 30 min. After another wash step, detection is performed with diaminobenzidine (DAB) with H2O2 as substrate. The cells or sections are coverslipped on gelatin-coated slides with DPX (Fluka, Bornem, Belgium).

For fluorescent staining, cells or sections are incubated overnight with the primary antibody diluted in PBST, 10% sodium azide and 10% goat serum. After three PBST rinses, cells or sections are incubated for 2 hours at room temperature with goat anti-mouse IgG-Alexa 633 (diluted 1: 500, Invitrogen Molecular Probes). Cells or sections are again rinsed with PBST and mounted on microscope slides with polyvinyl alcohol (Mowiol; Merck, La Jolla, Calif., USA).

DAB staining of transgene expression in cells or sections is visualized by a Leica DMR optical microscope. Fluorescence staining is visualized by confocal microscopy using a LSM510 Laser Scanning Microscope (Zeiss, Zaventem, Belgium).

IHC for TSC1 and GFAP: Primary antibodies are: polyclonal antibodies against TSC1 (1:1000) purchased from Cell Signaling Technology (Danvers, Mass.); Cy3-conjugated mouse monoclonal anti-glial fibrillary acidic protein (GFAP) at 1:2000 (Sigma, St Louis, Mo., USA). Briefly, selected cells or coronal sections are blocked for 2 hours at room temperature in PBS with 3% BSA, 0.1% Triton X-100, 5% normal rabbit serum and 5% normal mouse serum, then incubated overnight with primary antibody against TSC1 diluted in PBS with 3% BSA and 0.1% Triton X-100 at 4° C., then washed and incubated with secondary antibodies ALEXA 488 goat anti-rabbit IgG (Molecular Probes) in PBS with 1% normal goat serum and 0.1% Triton X-100 for 2 hours at room temperature. After being washed three times in darkness, cells or sections are incubated with Cy3-conjugated mouse monoclonal anti-glial fibrillary acidic protein (GFAP) in PBS with 1% normal mouse serum and 0.1% Triton X-100 for 2 hours at room temperature. Labeled cells or sections are viewed by a Zeiss fluorescence microscope and associated software. Images (0.3- to 1.0-mm-wide slices) are captured and analyzed for localization of TSC1 and GFAP. Two-color merged images can be analyzed to determine the extent of colocalization of TSC1 and GFAP.

IHC for either TSC1 or GFAP alone can be performed as above, using only the steps required for the antigen of interest.

Western blotting: Western blots are used herein to analyze protein expression levels. Samples are prepared according to known methods. For example, cells or tissue are harvested and homogenized immediately by sonication in lysis buffer containing 1% Triton-x-100, 50 mM Tris pH 7.5, 150 mM NaCl, 1 mM EGTA, 1 mM EDTA, 1 mM NaF, 1 mM sodium vandidate, and a cocktail of proteinase inhibitors. Lysates are boiled for 10 min with sample buffer containing 2% SDS and are stored at −20° C. Lysates are electrophoresed on SDS-PAGE mini-gels (Novex, San Diego, Calif.), and the separated proteins are transferred to Immobilon-P membranes (Millipore Corp., Bedford, Mass.).

Western blots are performed with primary antibodies can include: polyclonal anti-TSC1 (1:1000) purchased from Cell Signaling Technology (Danvers, Mass.); anti-GFAP (ab4674, 1:10,000; Abcam, San Francisco, Calif.); anti-V5 (1:5000, Invitrogen); eGFP (A11122, 1:1000, Invitrogen); monoclonal anti-α-tubulin (1:50,000) as loading control from Sigma Chemical Co (St. Louis, Mo.). Horseradish peroxidase (HRP)-linked secondary antibodies (1:10,000 dilution, Cell Signaling Technology, Danvers, Mass.) and the enhanced chemilluminance reagents (ECL; supersignal, Pierce, Rockford, Ill.) are used for visualization. The expression level of proteins are normalized against the expression level of α-tubulin. To semiquantitate immunoblots, the films are digitally scanned into Adobe Photoshop and the selected bands subjected to pixel analysis by using the UN-SCAN-IT software (Silk Scientific, Orem, Utah) after confirmation that each antibody is in the linear range for the protein of interest by a dose-response analysis.

Analysis of phosphorylation of T389, T229 S6kinase (S6K); 4E-BP1, and/or AKT: Analysis of protein phosphorylation is an extension of the western blot technique described above. Phosphorylation analysis includes protein preparation in a buffer comprising the phosphatase inhibitor okadaic acid in the lysis buffer. The proteins are electrophoresed and blotted as for a western blot. During the detection step, the phosphorylated protein is detected by a primary antibody directed against a short peptide sequence that includes the phosphorylated amino acid and compared to detection of the total protein, i.e., non-phospho-+phospho-protein. Antibodies used herein include, but are not limited to: S6K (phospho T389) (ab32359, abcam), S6K (phospho T229) (ab5231, abcam), and S6K (total) (ab36864, abcam); 4E-BP1 (phospho S65) (ab81297, abcam) and 4E-BP1 (total) (ab2606, abcam); and AKT (phospho 5473) (ab66138, abcam) and AKT (total) (ab74117, abeam). Secondary antibodies used are appropriate to the primary antibody. Detection is done by standard methods (supra).

### Example 2

This example describes transfection of CRL-2534 cells with AAV9-hTSC1-V5.

Human astrocytoma CRL-2534 cell line is purchased from ATCC. The cell line is stored and propogated according to the ATCC's protocol. In brief, the adherent cells are cultured in RPMI-1640 Medium with 10% fetal bovine serum, and incubated in 5% CO₂ atmosphere at 37° C.

High-titer AAV9-hTSC1-V5 viral particles with titer of 1×10¹³ genome copy per ml are purchased from ReGenX Bioscience and stored according to ReGenX Bioscience protocol. A few drops of viral particles are thawed and are added to the 80% confluent CRL-2534 astrocytotoma culture overnight for transfection.

### Example 3

This example describes experiments to determine the efficiency of AAV9-hTSC1-V5 transfection of CRL-2534 cells.

CRL-2534 cells are transfected with AAV9-hTSC1-V5 (see, e.g., Example 2). After overnight incubation with virus, the cells are washed with PBS and incubated for another 2 days. To evaluate AAV9-hTSC1-V5 viral transfection efficiency, the reporter protein expression and the duration of the reporter protein expression in human astrocytoma CRL-2534 culture is evaluated as follows.

Cultures of transfected astrocytotoma cells are analyzed by IHC as described in, e.g., Example 1. The numbers of both V5-positive and V5-negative cells are counted in random microscopic fields. The viral transfection efficiency is calculated as number of cells with V5 expression over total cells in the pre-defined areas. Duration of reporter protein expression is assessed by counting V5-positive and V5-negative cells by fluorescence microscopy every two weeks.

### Example 4

This example describes experiments on CRL-2534 cells transfected with AAV9-hTSC1-V5.

Mouse astrocyte type III CRL-2534 cells are obtained from the ATCC and maintained according to ATCC protocols. CRL-2534 cells are transfected with the AAV9-hTSC1-V5 vector. IHC with an anti-V5 antibody (Invitrogen, Inc., Carlsbad Calif.) is performed to assess hTSC1 protein expression (see, e.g., methods described in Example 1). Repeated IHC at regular intervals assesses the intensity, duration, and efficiency of hTSC1 expression over time. Simultaneous Western blots for hTSC1 and V5 levels in cultures, with and without AAV9-hTSC1-V5 transfection, confirm recombinant hTSC1 expression.

Western blots with antibodies against T389 and/or T229 S6kinase (S6K); 4E-BP1, and/or AKT assess the functional effects of hTSC1 protein expressed from AAV9-hTSC1-V5 on cell signaling. Comparison of the ratio of phosphorylated S6K over total S6K between cultures with and without AAV9-hTSC1-V5 represents the inhibition of S6K phosphorylation expected in cells with hTSC1. Additionally, the phosphorylation states of T229 S6K, 4E-BP1, and AKT are assessed by Western blotting. The influence on signal transduction in cultured cells by AAV9-hTSC1 without the V5 tag is also assessed.

### Example 5

This example describes culturing TSC1-knockout hippocampal cells.

A Tsc1 GFAPCKO primary hippocampal astrocyte culture is established as previously described (Cormier et al., 2001). Briefly, a hippocampal mass culture is prepared by dissecting the hippocampi from 2-4 P1 Tsc1 GFAPCKO mice and, separately, from WT mice, dissociating the cells, and plating them in petri dishes, then replating on coverslips. The culture medium is washed off of the culture hippocampal astrocyte cells with artificial cerebrospinal fluid consisting of 119 mM NaCl, 2.5 mM KCl, 2.5 mM CaCl₂, 1.3 mM MgSO₄, 1 mM NaH₂PO₄, 26.2 mM NaHCO₃ and 11 mM glucose, saturated with 95% O₂-5% CO₂. The cells are then loaded with Fluo 4-AM, a nonratioable Ca2+ indicator, by transferring an astrocyte coverslip to a petri dish containing artificial cerebrospinal fluid containing 10 μM of the Fluo 4-AM-indicator.

### Example 6

This example describes experiments to determine the AAV9-hTSC1-V5 viral transfection efficiency, the TSC 1 protein expression, and the duration of protein expression in primary astrocyte culture from Tsc1GFAPCKO hippocampal astrocyte culture cells.

Tsc1 GFAPCKO hippocampal astrocyte culture cells are generated using methods described in, e.g., Example 5. Cultured Tsc1GFAPCKO hippocampal astrocyte cells are transfected with the AAV9-hTSC1-V5 vector, as in, e.g., Examples 2-3.

The viral transfection efficiency, protein expression and its duration in primary hippocampal astrocyte culture are assessed and analyzed biweekly, using methods described in, e.g., Examples 1 and 3.

TSC1 protein level in WT and Tsc1GFAPCKO hippocampal primary astrocyte cultured cells transfected with AAV9-hTSC1-V5 is determined with routine western blot analysis, using methods from, e.g., Example 1. In brief, culture cells are harvested two days after viral transfection, and homogenized immediately by sonication in lysis buffer containing 1% Triton-x-100, 50 mM Tris pH 7.5, 150 mM NaCl, 1 mM EGTA, 1 mM EDTA, 1 mM NaF, 1 mM sodium vandidate, and a cocktail of proteinase inhibitors. Lysates are boiled for 10 min with sample buffer containing 2% SDS and are stored at −20° C. Lysates are electrophoresed on SDS-PAGE mini-gels (Novex, San Diego, Calif.), and the separated proteins are transferred to Immobilon-P membranes (Millipore Corp., Bedford, Mass.). Western blots are performed with primary polyclonal antibodies against TSC1 (1:1000) purchased from Cell Signaling Technology (Danvers, Mass.), and with monoclonal antibody against α-tubulin (1:50,000) as loading control from Sigma Chemical Co (St. Louis, Mo.). Horseradish peroxidase (HRP)-linked anti-rabbit secondary antibody (1:10,000 dilution, Cell Signaling Technology, Danvers, Mass.) and the enhanced chemilluminance reagents (ECL; supersignal, Pierce, Rockford, Ill.) are used for visualization. The expression level of proteins are normalized against the expression level of α-tubulin. To semiquantitate immunoblots, the films are digitally scanned into Adobe Photoshop and the selected bands subjected to pixel analysis by using the UN-SCAN-IT software (Silk Scientific, Orem, Utah) after confirmation that each antibody is in the linear range for the protein of interest by a dose-response analysis.

A fluorescence microscopy assessment of V5 protein expression (using IHC) and western blot assessment of TSC1 protein level is performed biweekly in Tsc1^{GFAP}CKO hippocampal primary astrocyte cultured cells transfected with AAV9-hTSC1-V5 to assess the duration of the protein expression for up to six months, according to methods in, e.g., Examples 1 and 3. Statistical comparisons are made with one-way ANOVA to identify statistically significant differences (P<0.05).

### Example 7

This example describes experiments on cultured Tsc1GFAPCKO hippocampal astrocyte cells to assess mGluR5 modulation of intracellular Ca2+ levels.

Tsc1 GFAPCKO hippocampal astrocyte cell cultures are generated, as in e.g. Example 5 and Cormier et al., 2001. Primary cultures are prepared from P1 Tsc1GFAPCKO and WT mouse pups and studied at 7-10 days.

Cultured hippocampal astrocyte cells are transduced with Lenti-GFAP-hTSC1-T2A-eGFP. Fluorescent microscopy is performed on an inverted microscope for Ca²⁺ imaging with Fura-2 (Invitrogen), using manufacturer's protocols.

Astrocytic glutamate receptors are first activated by bath application of the mGluR5 agonist ACPD. Results are compared for astrocytes from WT, Tsc1GFAPCKO, and Tsc1GFAPCKO+transduction by Lenti-GFAP-hTSC1-T2A-eGFP. Mixed cultures of astrocytes and neurons are also used, to test the feasibility of activating astrocytic mGluR5 by synaptic glutamate released by stimulating individual neurons with a micro-electrode.

### Example 8

This example describes methods to introduce AAV9-hTSC1-T2A-V5 into mice.

High-titer AAV9-hTSC1-V5 virion (1×10¹³ genome copies per ml) (ReGenX Bioscience, Philadelphia, Pa., USA, e.g., Example 1) is intravenously injected into mouse via tail vein at 3 weeks and 6 weeks of age as previously described (Faust et al., 2009). Briefly, the vector solution is drawn into a 3/10 cc 30 gauge insulin syringe. Mice are placed in a restraint that positions the mouse tail in a lighted, heated groove. Virus injections are in a total volume of 50 ml of PBS supplemented with 0.001% pluronic-F68. The tail is swabbed with alcohol then injected intravenously with a 1 ml viral solution containing a mixture of PBS and 1×10¹² DNase-resistant particles of AAV9-hTSC1-V5 (ReGenX Bioscience) for a mouse of 3 weeks old, and 5 ml solution containing a mixture of PBS and 5×10¹² viral particles for a mouse of 6 weeks old. The needle is inserted into the vein and the plunger is manually depressed. A correct injection is verified by noting blanching of the vein. After the injection, mice are returned to their cages. Both WT and Tsc1GFAPCKO mice are used in this study.

### Example 9

This example describes experiments that determine the efficiency of injected AAV9-hTSC1-V5 targeting to mouse brain tissues.

Mice are injected with AAV9-hTSC1-V5 according to methods described in, e.g., Example 7. When mice reach 10 weeks of age, or after video-EEG recording, histology and immunohistochemisty studies are performed.

Mice are transcardially perfused with PBS followed by 4% paraformaldehyde in 0.1 M phosphate buffer, pH 7.4. The brains are postfixed in the same fixative at 4° C. overnight, cryoprotected in 30% sucrose for 24 h, and are frozen in optimal cutting temperature compound. Serial coronal sections are cut at 10 μm thickness on a cryostat. One out of every five sections is examined for V5 expression by IHC.

TSC1 protein expression and colocalization with brain astrocytes in the Tsc1 GFAPCKO and WT are is examined via IHC. Primary antibodies are: polyclonal antibodies against TSC1 (1:1000) purchased from Cell Signaling Technology (Danvers, Mass.); Cy3-conjugated mouse monoclonal anti-glial fibrillary acidic protein (GFAP) at 1:2000 (Sigma, St Louis, Mo., USA), and/or anti-V5 monoclonal antibody (R960-25, Invitrogen). Brains from 10 week old mice are fixed by perfusion with 4% paraformaldehyde and are additionally or simultaneously studied by IHC using anti-V5 antibodies on 50 μm thick coronal brain sections cut with a microtome (Lobbestael et al., 2010). IHC is performed according to, e.g., Example 1.

Sections are compared between animals with and without AAV9-hTSC1-V5 injection. The hTSC1, GFAP and V5 expression patterns, intensities, and durations in Tsc1GFAPCKO mouse brains and correlate them with the dosage and timing of IV viral injections. Astrocyte transduction is assessed in brain slices with double-immunostaining for hTSC1, or V5, and mouse GFAP. Anti-TSC1, or anti-V5, antibodies may be used for western blots and/or immunoprecipitation to quantitate AAV9 transduction.

For each mouse, the number of V5-positive or TSC1 and GFAP double-stained cells in every fifth section is counted and summed, covering the regions of interest in its rostrocaudal extension. The entire dentate gyms, caudal retrosplenial/cingulate cortex, containing the most caudal extent of the dentate gyms, extending medially to the subiculum and laterally to the occipital cortex, are sampled. These sums allow quantitative comparisons among the transduced brain areas, optimal dosages, and genotypes, although they do not reflect the total number of transduced cells in vivo. For evaluating TSC1 expression duration in mouse brain after vector transduction, either V5 florescence histology or IHC for TSC1 is performed bi-monthly for the first 6 months, then monthly afterwards. In addition, western blot studies with an anti-V5 primary antibody are used to characterize expression in the various areas of mice brain after vector injection is performed in a similar manner as described in, e.g., Example 5. The data is analyzed by ANOVA and adjusted P value<0.05 is considered statistically significant. Dosages with highest transduction efficacy are selected for behavioral and EEG studies (infra)

### Example 10

This example describes experiments to assess the sites of hTSC1 transfection in tail-vein injected mice.

The tail veins of Tsc1GFAPCKO and WT mice are injected with a mixture of AAV9-hTSC1 and AAV9-eGFP (see, e.g., Example 7). The expression pattern of eGFP is assessed by IHC and/or fluorescence microscopy as an estimate for hTSC1 expression patterns.

### Example 11

This example describes experiments to determine the general health and behavior of Tsc1GFAPCKO mice.

One week prior to the beginning of the experiment, animals are acclimated to the experimental facility by being transported to and housed in the behavioral laboratory where the experiments are performed. To minimize the confounding of genotypes with other variables, such as lighting condition, noise, airflow in the experimental room, the order of mice group to be assigned to each run alternates, with 10 to 12 mice from each genotypes.

Tsc1GFAPCKO and WT mice of 4 through 16 weeks of age are screened for general health and behaviors. Each mouse is weighed and measured, then placed into an empty cage and observed for 1 min by a human observer blinded to genotype. The presence of several behavioral responses is recorded (i.e., wild running, freezing, licking, jumping, sniffing, rearing, movement throughout the cage, and defecation). The general sensorimotor responses of the Tsc1GFAPCKO and WT mice are evaluated over two test sessions (Schaefer et al., 2000) with first session commencing at 09:00 hr, and the second session 3 hours later, including Inclined Screen, Ledge, Walking Initiation.

To assess Tsc1GFAPCKO and WT emotionality (Zeng et al., 2008; Crawley, 2000), a Platform test is conducted by placing the animal in the center of a brightly lit platform (20 cm): the number of freezing, fecal boli deposited, urination, the latency to move to the edge, and the number of times the mouse reaches its head over the edge (nose pokes) are recorded by a human counter blinded to the genotypes.

A number of mouse home cage behaviors (Crawley, 2008) are observed and scored by a human observer blinded to genotype. Presence or absence of huddling is scored when mice sleep together in the home cage during light cycle. Nest building is tested after providing clean nesting material. Nest building is quantified by measuring the time taken to start and finish the nest, the nest weight, the nest height and the diameter of the nest.

Parental care of pups may be scored for the time spent within the nest. The frequency of searching for pups and latency to retrieve pups after the pups are removed from the nest is scored. Maternal behaviors are scored for number of bouts, time spent licking pups, time spent crouching over pups, and time spent nursing.

Postural reflexes (Zeng et al., 2008) are evaluated by determining if the mouse splays its limbs when in a cage that is quickly lowered and moved from side to side.

Juvenile play behavior in 4 to 6 week old mice is tested for 10 minutes with a stimulus mouse of same age pretreated with a sedative. The test animal is scored for play solicitation on three measures: number of crossover, play grooms, and running toward or away from the stimulus mouse.

Social interaction between two mice who have not met before is scored in a familiar versus an unfamiliar open-field environment by a blinded observer. Sniffing, following, grooming, kicking, mounting, jumping on, wrestling and other forms of physical contact are quantified over a 10-minute test period. Subsequent social interaction with a familiar mouse met 5 minutes before is scored as an index for social recognition.

Aggression is assessed with the Resident—Intruder test. A 5-minute fighting test session is scored in the home cage of the test mouse, including frequency of occurrence of behaviors, including general body sniffs, anogenital sniffs, following, chasing, threat, tail rattle, attacks, number of bites, location of bites, escapes, upright subordinate posture, body contact, and allogrooming. If attacks and bites become severe, the session are terminated. Behavior data is analyzed using two-way ANOVA model, and p<0.05 is considered to be statistically significant.

Age of the mice at natural death is recorded.

### Example 12

This example describes visual monitoring of Tsc1GFAPCKO mice for seizure or seizure-like activity.

Behavioral monitoring is done to ensure that mice have continuous seizures: continuous until the onset of status epilepticus and every 30-60 min thereafter to monitor whether SE was maintained. Seizure progression includes an initial stage with immobility followed by bouts of scratching behavior, hyperactivity and ataxia, focal limb myoclonus, and tonic or tonic-clonic seizures without interictal recovery. The visual epileptic activities are correlated with activities by video and EEG recordings as described in, e.g., Examples 13-17.

### Example 13

This example describes materials and methods to monitor seizures and seizure-like activity in Tsc1GFAPCKO mice.

To characterize the electroencephalographic (EEG) correlates of seizures in mice in which the Tsc1 gene has been conditionally inactivated in glial cells Tsc1 GFAPCKO mice, young male mice are used for video-EEG monitoring with the apparatus from Pinnacle Technology (Lawrence, Kans.), as previously described (Chung et al., 2009). Mice selected for video-EEG are male, minimizing gender influence on Tsc1 GFAPCKO seizure activities (Engel Jr. & Pedley, 2008; Janszky, 2004), and are 10 weeks of age, when most Tsc1GFAPCKO mice are still viable but are developing progressively visible and EEG-detectable epilepsy (Erbayat-Altay, Zeng, Xu, Gutmann, & Wong, 2007; Uhlmann et al., 2002; Zeng, Xu, Gutmann, & Wong, 2008).

The EEG recording apparatus is an 8400-K1-SE4 model, which is a new and compact system that does not require any additional acquisition cards or amplifiers; instead, data are transferred to a digital computer via a USB connection, which also provides power to the recording apparatus. Video from a Pinnacle 8236 infrared video camera that captures mouse activity under both light and dark conditions is synchronized with the EEG data to allow correlation of behavior with brain electrical activity.

Surgeries to install EEG recording electrodes are performed according to Pinnacle's protocol. Briefly, male mice 9 weeks of age are anesthetized by intraperitoneal injection of ketamine (80 mg/kg), and xylazine (9 mg/kg) and secured in a stereotaxic frame (David Kopf Instruments, Tujunga, Calif.). Under sterile conditions, an incision is made in the scalp to expose the skull, and four holes the size of a 23 gauge needle are drilled through the skull to the surface of the dura mater. These holes accommodate a prefabricated mouse headmount, which is fastened to the skull with stainless steel screws (Small Parts, Miami Lakes, Fla.). Two epidural EEG screw electrodes are placed 1 mm anterior to the bregma and two are placed 7 mm anterior to the bregma, each being 1.5 mm lateral to the central sulcus, using the headmount frame as a guide to ensure proper placement, spacing, and positioning in the frontal and parietal cortices, as previously described (Chung et al., 2009). The headstage is secured to the skull with dental acrylic; the loose skin is sutured around the implant, and at least 7 days are allowed before beginning a 48 hour EEG data collection epoch in mice that are 10 weeks of age.

Twelve pairs of young male mice at 9 weeks of age from each genotype undergo epidural electrode implantation surgery and video-EEG monitoring. With one video-EEG monitoring apparatus, video-EEG monitoring is performed on one mouse each time, with Tsc1 GFAPCKO and WT on alternate days to minimize environmental factors. A 24-hour epoch of continuous video-EEG data is obtained from each mouse, after one week recovery from the epidural electrode implantation surgery. The clinical and electrographic characteristics of seizures from two genotypes are analyzed and compared using the Persyst Advanced EEG suite, including the measures for average frequency, number, amplitude, and duration of EEG epileptic activities, interictal EEG grade, interictal EEG spike frequency, and average number, frequency, amplitude, pattern of video seizures. Data are statistically analyzed with Systat to determine if the mean differences between Tsc1GFAPCKO and WT in the number of seizures per 24-hour epoch, duration of each seizure, and grade of interictal background activity are significant at the p≦0.05 level.

### Example 14

This example describes methods to capture synchronized EEG signals and video recording of seizures and seizure-like activity in mice.

WT and Tsc1GFAPCKO mice which have been previously fitted with EEG monitors as in, e.g., Example 13, are placed individually in 10 inch diameter, cylindrical recording chambers and allowed ad libitum access to food and water. EEG signals are collected from the headstage with a 100 preamplifier, passed to the 100 main amplifier by a tether/swivel/commutator, filtered with a 0.5 Hz high pass, a 50 Hz low pass, and a 60 Hz digital notch filter, sampled at 400 Hz, digitized at 14 bit, stored, and analyzed on a PC running Pinnacle Acquisition Laboratory (PAL) software. A video camera is placed to ensure all areas of the cylindrical recording chambers where the mouse undergoes EEG monitoring is captured. Video data are collected at 640×480 resolution, 24 bit, and 30 Hz, time-stamped, and stored on the computer's hard disk in DIVX format by the PAL software, which synchronously analyzes video and electrographic data.

### Example 15

This example describes analysis of EEG data.

The clinical and electrographic characteristics of seizures exhibited by Tsc1 GFAPCKO mice are analyzed quantitatively with published methods (Erbayat-Altay, Zeng, Xu, Gutmann, & Wong, 2007; Zeng, Xu, Gutmann, & Wong, 2008). Spontaneous seizures in Tsc1GFAPCKO mice are electrographically identified as repetitive, rhythmic (2-8 Hz), high-amplitude (>2 fold above background) sharp-wave activity lasting longer than 10 seconds with an initial onset of a tonic, repetitive spike discharge followed by a progressive evolution in spike amplitude and frequency that usually culminates in a bursting pattern and postictal suppression (Erbayat-Altay, Zeng, Xu, Gutmann, & Wong, 2007; Shin, Brager, Jaramillo, Johnston, & Chetkovich, 2008). Other types of seizures, such as spike-wave events (Chung et al., 2009) lasting less than 10 s are identified separately from the Tsc1GFAPCKO type seizure. Electrographic seizure activity is visually confirmed on the synchronized video recording to be free of environmental disturbance. Seizure activity is monitored over a 24-hour epoch, and each event is characterized by type of event, duration, and time since previous event. Interictal spikes are defined as fast (<200 ms) epileptiform waveforms that are at least twice the amplitude of the background activity. The scoring of the interictal background activity is based on a four-grade scale: 1—normal background activity (±6-8 Hz sinusoidal theta rhythm), no epileptiform spikes; 2—mostly normal background activity, few epileptiform spikes; 3—mostly abnormal background activity, many spikes; 4—burst-suppression pattern. An average score for spike frequency (spikes/minute) and background activity (grade 1-4) is calculated for each 24-h epoch (Griffey et al., 2006).

### Example 16

The general behaviors and epileptic activities in Tsc1GFAPCKO with AAV9-hTSC1-V5 administration are analyzed and compared with that in Tsc1GFAPCKO without the treatment. The optimal treatment dosage, time course, and the duration of AAV9-hTSC1-V5 gene therapy for the epilepsy by astrocyte TSC1 deficiency is determined. In addition, the alterations in Tsc1GFAPCKO general behaviors and epileptic activities with the gene therapy in correlation to the patterns of astrocytic TSC1 expression in various areas of Tsc1GFAPCKO brain are analyzed as well.

### REFERENCES

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.
Altimus, Güler, Villa, McNeill, Legates, Hattar, et al. (2008). Rods-cones and melanopsin detect light and dark to modulate sleep independent of image formation. Proceedings of the National Academy of Sciences of the United States of America, 105(50), 19998-20003. doi: 10.1073/pnas.0808312105.
Au, Ward and Northrup. (2008). Tuberous sclerosis complex: disease modifiers and treatments. Current Opinion in Pediatrics, 20, 628-633. doi: 10.1097/MPO.0b013e328318c529
Camfield, Camfield, Smith, Dooley, & Smith. (2002). Long-term outcome is unchanged by antiepileptic drug treatment after a first seizure: a 15-year follow-up from a randomized trial in childhood. Epilepsia, 43(6), 662-3. Retrieved from http://www.ncbi.nlm.nih.gov/pubmed/12060028.
Cormier, Mennerick, Melbostad, and Zorumski. (2001). Basal levels of adenosine modulate mGluR5 on rat hippocampal astrocytes. Glia. 33(1), 24-35.
Crawley. (2008) Behavioral phenotyping strategies for mutant mice. Neuron. 57(6), 809-818.
Daya and Berns. (2008). Gene therapy using adeno-associated virus vectors. Clin. Microbiol. Rev. 21(4), 583-593.
Erbayat-Altay, Zeng, Xu, Gutmann, & Wong. (2007). The natural history and treatment of epilepsy in a murine model of tuberous sclerosis. Epilepsia, 48(8), 1470-6. doi: 10.1111/j.1528-1167.2007.01110.x.
Ess, Kamp, Tu and Gutmann. Developmental origin of subependymal giant cell astrocytoma in tuberous sclerosis complex. (2005). Neurology. 64, 1446-1449.
Foust and Kaspar. (2009) Over the barrier and through the blood: to CNS delivery we go. Cell Cycle. 8(24):4017-8
Foust, Nurre, Montgomery, Hernandez, Chan, and Kaspar. (2009) Intravascular AAV9 preferentially targets neonatal neurons and adult astrocytes. Nat. Biotechnol. 27(1):59-65. Epub 2008 Dec. 21.
Griffey, Wozniak, Wong, Bible, Johnson, Rothman, Wentz, Cooper, and Sands. (2006). CNS-directed AAV2-mediated gene therapy ameliorates funcation deficits in a murine model of infantile neuronal ceroid lipofuscinosis. 13(3), 538-547.
Gutmann, Zhang, Hasbani, Goldberg, Plank, and Henske. (2000). Expression of the tuberous sclerosis complex gene products, hamartin and tuberin, in central nervous system tissues. Acta Neuropahology 99.223-230.
Helibronn and Weger. (2010). Viral vectors for gene transfer: current status of gene therapeutics. Handbook of Experimental Pharmacology 197:143-170.
Holmes, Stafstrom and the Tuberous Sclerosis Study Group. (2007) Tubersous sclerosis complex and epilepsy: recent developments and future challenges. Eiplepsia 48(4), 617-630.
Jin, Wienecke, Xiao, Maize Jr, DeClue, and Yeung. (1996). Suppression of tumorigenicity by the wild-type tuberous sclerosis 2 (Tsc2) gene and its C-terminal region. PNAS 93(17), 9154-9159.
Lobbestael, Reumers, Ibrahimi, Paesen, Thiry, Gijsbers, Van den Haute, Debyser, Baekelandt, and Taymans (2010). Immunohistochemical detection of transgene expression in the brain using small epitope tags. BMC Biotechnol. 10, 16. doi: 10.1186/1472-6750-10-16.
Miloloza, Rosner, Nellist, Halley, Bernaschek, and Hengstschläger. (2000). The TSC1 gene product, hamartin, negatively regulates cell proliferation. Hum. Mol. Genet. 9(12), 1721-1727.
Napolioni & Curatolo. (2008). Genetics and molecular biology of tuberous sclerosis complex. Current genomics, 9(7), 475-87. doi: 10.2174/138920208786241243.
Nellist, van Slegtenhorst, Goedbloed, van den Ouweland, Halley, and van der Sluijs. (1999) Characterization of the cytosolic tuberin-hamartin complex. Tuberin is a cytosolic chaperone for hamartin. J. Biol. Chem. 274(50), 35647-35652.
Povey, Burley, Attwood, Benham, Hunt, Jeremiah, Franklin, Gillett, Malas, Robson, et al. (1994). Two loci for tuberous sclerosis: on 9q34 and one on 16p13. Ann. Hum. Genet., 58(pt. 2), 107-127.
Riban, Fitzsimons and During. (2009). Gene therapy in epilepsy. Eiplepsia 50(1), 24-32.
Rogawski. (2006). Molecular targets versus models for new antiepileptic drug discovery. Epilepsy research, 68(1), 22-8. doi: 10.1016/j.eplepsyres.2005.09.012.
Sampson. (2009). Therapeutic targeting of mTOR in tubersou sclerosis. Biochem. Soc. Trans. 37, 259-264. doi: 10.1042/BST0370259
Schuele & Liiders. (2008). Intractable epilepsy: management and therapeutic alternatives. Lancet neurology, 7(6), 514-24. doi: 10.1016/S1474-4422(08)70108-X.
Shin, Brager, Jaramillo, Johnston, & Chetkovich. (2008). Mislocalization of h channel subunits underlie h channelopathy in temporal lobe epilepsy. Neruobiol. Dis. 32(1), 26-36.
Sosunov, Wu, Weiner, Mikell, Goodman, Crino, et al. (2008). Tuberous sclerosis: a primary pathology of astrocytes? Epilepsia, 49 Suppl 2, 53-62. doi: 10.1111/j.1528-1167.2008.01493.x.
Uhlmann, Wong, Baldwin, Bajenaru, Onda, Kwiatkowski, et al. (2002). Astrocyte-specific TSC 1 conditional knockout mice exhibit abnormal neuronal organization and seizures. Ann. Neurol., 52, 285-296.
van Slegtenhorst, Nellist, Nagelkerken, Cheadle, Snell, van den Ouweland, Reuser, Sampson, Halley, and van der Sluijs. (1998). Interaction between hamartin and tuberin, the TSC1 and TSC2 gene products. Hum. Mol. Genet. 7(6), 1053-1057.
Wienecke, König, and DeClue. (1995). Identification of tuberin, the tuberous sclerosis-2 product. Tuberin possesses specific Rap1GAP activity. J. Biol. Chem. 270(27), 16409-16414.

Wong. (2008). Mechanisms of epileptogenesis in tuberous sclerosis complex and related malformations of cortical development with abnormal glioneuronal proliferation. Epilepsia, 49(1), 8-21. doi: 10.1111/j.1528-1167.2007.01270.x.
Zeng, Bero, Zhang, Holtzman, & Wong. (2010). Modulation of astrocyte glutamate transporters decreases seizures in a mouse model of Tuberous Sclerosis Complex. Neurobiology of Disease, 37(3), 764-771. Elsevier Inc. doi: 10.1016/j.nbd.2009.12.020.
Zeng, Xu, Gutmann and Wong. (2008). Rapamycin prevents epilepsy in a mouse model of tuberous sclerosis complex. Ann. Neurol. 63(4), 444-453.

1. A method of treating Tuberous Sclerosis Complex (TSC), the method comprising
administering to a subject in need thereof a therapeutically effective amount of a composition comprising an adeno-associated virus (AAV) vector, the AAV vector comprising at least one polynucleotide encoding a TSC1 or a TSC2, or variant thereof;
wherein TSC1 or TSC2 is expressed in a plurality of cells of the subject.

2. The method of claim 1, wherein the AAV vector comprises a polynucleotide encoding a TSC1, or variant thereof, having a nucleic acid sequence selected from the group consisting of:
SEQ ID NO: 2, SEQ ID NO: 6, SEQ ID NO: 14, SEQ ID NO: 16, SEQ ID NO: 18, SEQ ID NO: 20, or SEQ ID NO: 22;
a nucleic acid sequence having at least about 90% identity to SEQ ID NO: 2, SEQ ID NO: 6, SEQ ID NO: 14, SEQ ID NO: 16, SEQ ID NO: 18, SEQ ID NO: 20, or SEQ ID NO: 22 and encoding a polypeptide having hamartin activity;
a nucleic acid sequence encoding a polypeptide of SEQ ID NO: 1, SEQ ID NO: 5, SEQ ID NO: 13, SEQ ID NO: 15, SEQ ID NO: 17, SEQ ID NO: 19, or SEQ ID NO: 21; and
a nucleic acid sequence encoding a polypeptide having at least about 90% identity to SEQ ID NO: 1, SEQ ID NO: 5, SEQ ID NO: 13, SEQ ID NO: 15, SEQ ID NO: 17, SEQ ID NO: 19, or SEQ ID NO: 21 and having hamartin activity.

3. The method of claim 1, wherein the AAV vector comprises a polynucleotide encoding a TSC2, or variant thereof, having a nucleic acid sequence selected from the group consisting of:
SEQ ID NO: 4, SEQ ID NO: 8, SEQ ID NO: 24, SEQ ID NO: 26, SEQ ID NO: 28, or SEQ ID NO: 30;
a nucleic acid sequence having at least about 90% identity to SEQ ID NO: 4, SEQ ID NO: 8, SEQ ID NO: 24, SEQ ID NO: 26, SEQ ID NO: 28, or SEQ ID NO: 30 and encoding a polypeptide having tuberin activity;
a nucleic acid sequence encoding a polypeptide of SEQ ID NO: 3, SEQ ID NO: 7, SEQ ID NO: 23, SEQ ID NO: 25, SEQ ID NO: 27, or SEQ ID NO: 29; and
a nucleic acid sequence encoding a polypeptide having at least about 90% identity to SEQ ID NO: 3, SEQ ID NO: 7, SEQ ID NO: 23, SEQ ID NO: 25, SEQ ID NO: 27, or SEQ ID NO: 29 and having tuberin activity.

4. The method of any of claims 1-3, wherein the at least one polynucleotide is operably linked to a heterologous promoter.

5. The method of any of claims 1-4, wherein the heterologous promoter is selected from the group consisting of: a glial fibrillary acidic protein (GFAP) promoter, a synapsin-1 (SYN) promoter, a Ca²⁺/calmodulin-dependent protein kinase II (CAMKII) promoter, a myelin basic protein (MBP) promoter, a nectin promoter, a myosin light polypeptide 2 (Myl-2) promoter, a SM22α gene promoter, a human cytomegalovirus immediate-early gene (CMV) promoter, and a human ubiquitin 6 (U6) promoter.

6. The method of claim 4, wherein the heterologous promoter is a glial fibrillary acidic protein (GFAP) promoter.

7. The method of any of claims 1-6, wherein the administering comprises intravenous administration.

8. The method of any of claims 1-7, wherein the subject in need of treatment displays at least one symptom selected from the group consisting of: brain tubers, brain tumors, subependymal nodules, subependymal giant cell astrocytomas, vascular stromas, peripheral nervous system tumors, retinal hamartomas, seizures, mental retardation, learning disabilities, behavior problems, autism, autism spectrum disorders, attention deficit hyperactivity disorder, and sleep disturbances.

9. The method of any of claims 1-8, wherein the subject in need of treatment displays at least one symptom selected from the group consisting of: renal lesions caused by angiomyolipomas, simple cysts, polycystic kidney disease, renal-cell carcinoma, renal lymphangiomyomatosis, cardiac lesion caused by cardiac rhabdomyomas, dermatological lesions caused by hyperpigmented maculars, angiofibromas, fibrous plaques, papules, Shagreen patches, gingival fibromas, and pulmonary lesions caused by lymphangiomyomatosis.

10. The method of any of claims 1-9, wherein the AAV vector is an AAV9 vector.

11. The method of any of claims 1-10, further comprising measuring one or more of:
(i) the expression of TSC1 or TSC2, wherein at least one of the polynucleotides for TSC1 or TSC2 is comprised by the AAV vector;
(ii) the activity level of a polypeptide encoded in the AAV vector; or
(iii) the level of mTOR signaling in cells comprised by the subject.

12. The method of claim 11, further comprising comparing the expression of TSC1 or TSC2 in the subject being treated for TSC to the expression of TSC1 or TSC2 in a subject who is not in need of treatment for TSC.

13. The method of claim 11, further comprising comparing the activity level of a polypeptide encoded in the AAV vector or the activity level of mTOR signaling in cells comprised by the subject being treated for TSC to the activity level of a polypeptide encoded in the AAV vector or the activity level of mTOR signaling in a subject who is not in need of treatment for TSC.

14. An isolated polynucleotide molecule comprising:
(i) a polynucleotide comprising an AAV9 vector;
(ii) a polynucleotide encoding a TSC1 or a TSC2, or variant thereof; and
(iii) a promoter;
wherein the promoter is operably linked to the polynucleotide encoding a TSC1 or a TSC2, or variant thereof.

15. The isolated polynucleotide molecule of claim 14, wherein the polynucleotide encoding TSC1 or TSC2, or variant thereof, is selected from the group consisting of:
SEQ ID NO: 2, SEQ ID NO: 6, SEQ ID NO: 14, SEQ ID NO: 16, SEQ ID NO: 18, SEQ ID NO: 20, or SEQ ID NO: 22;
a nucleic acid sequence having at least about 90% identity to SEQ ID NO: 2, SEQ ID NO: 6, SEQ ID NO: 14, SEQ ID NO: 16, SEQ ID NO: 18, SEQ ID NO: 20, or SEQ ID NO: 22 and encoding a polypeptide having hamartin activity;
a nucleic acid sequence encoding a polypeptide of SEQ ID NO: 1, SEQ ID NO: 5, SEQ ID NO: 13, SEQ ID NO: 15, SEQ ID NO: 17, SEQ ID NO: 19, or SEQ ID NO: 21;
a nucleic acid sequence encoding a polypeptide having at least about 90% identity to SEQ ID NO: 1, SEQ ID NO: 5, SEQ ID NO: 13, SEQ ID NO: 15, SEQ ID NO: 17, SEQ ID NO: 19, or SEQ ID NO: 21 and encoding a polypeptide having hamartin activity;
SEQ ID NO: 4, SEQ ID NO: 8, SEQ ID NO: 24, SEQ ID NO: 26, SEQ ID NO: 28, or SEQ ID NO: 30;
a nucleic acid sequence having at least about 90% identity to SEQ ID NO: 4, SEQ ID NO: 8, SEQ ID NO: 24, SEQ ID NO: 26, SEQ ID NO: 28, or SEQ ID NO: 30 and encoding a polypeptide having tuberin activity;
a nucleic acid sequence encoding a polypeptide of SEQ ID NO: 3, SEQ ID NO: 7, SEQ ID NO: 23, SEQ ID NO: 25, SEQ ID NO: 27, or SEQ ID NO: 29; and
a nucleic acid sequence encoding a polypeptide having at least about 90% identity to SEQ ID NO: 3, SEQ ID NO: 7, SEQ ID NO: 23, SEQ ID NO: 25, SEQ ID NO: 27, or SEQ ID NO: 29 and encoding a polypeptide having tuberin activity.

16. The isolated polynucleotide molecule of any of claims 14-15, wherein the promoter is a human cytomegalovirus promoter.

17. The isolated polynucleotide molecule of claim 16, wherein the human cytomegalovirus promoter comprises a nucleic acid sequence of SEQ ID NO: 9, or 90% identity thereto and having cytomegalovirus promoter activity.

18. The isolated polynucleotide molecule of claim 14, wherein the sequence of the isolated polynucleotide comprises SEQ ID NO: 12.

19. A pharmaceutical composition comprising the isolated polynucleotide molecule of any of claims 14-18 and a pharmaceutically acceptable carrier or excipient.

20. A virion comprising the isolated polynucleotide of any of claims 14-18, wherein the virion is capable of delivering cargo to human cells.

21. The virion of claim 19, wherein the virion comprises AAV9 capsid or AAV9 capsid comprising mutations not found in naturally-occurring isolates of AAV9.

22. A cell comprising the isolated polynucleotide of any of claims 14-18.

23. Use of an isolated polynucleotide molecule for the treatment of Tuberous Sclerosis Complex (TSC), the isolated polynucleotide molecule comprising:
(a) the isolated polynucleotide molecule of any one of claims 14-18; or
(b) (i) a polynucleotide comprising an AAV9 vector, (ii) a polynucleotide encoding a TSC1 or a TSC2, or variant thereof, and (iii) a promoter, wherein the promoter is operably linked to the polynucleotide encoding a TSC1 or a TSC2, or variant thereof.

24. The use of isolated polynucleotide molecule in the manufacture of a medicament for the treatment of Tuberous Sclerosis Complex (TSC), the isolated polynucleotide molecule comprising:
(a) the isolated polynucleotide molecule of any one of claims 14-18; or
(b) (i) a polynucleotide comprising an AAV9 vector, (ii) a polynucleotide encoding a TSC1 or a TSC2, or variant thereof, and (iii) a promoter, wherein the promoter is operably linked to the polynucleotide encoding a TSC1 or a TSC2, or variant thereof.
